(19) United States
(12) Patent Application Publication
(10) Pub. No.: US 2016/0174532 A1
(43) Pub. Date: Jun. 23, 2016

(54) GENETICALLY MODIFIED RAT MODELS FOR PAIN

(71) Applicant: Transposagen Biopharmaceuticals, Inc., Lexington, KY (US)

(72) Inventors: Eric M. Ostertag, Lexington, KY (US); John Stuart Crawford, Lexington, KY (US); Karin Westlund High, Lexington, KY (US)

(21) Appl. No.: 14/979,760

(22) Filed: Dec. 28, 2015

Related U.S. Application Data
(63) Continuation of application No. 14/502,775, filed on Sep. 30, 2014, now abandoned.
Application No. 14/502,775 is a continuation of application No. 13/391,309, filed on Mar. 2, 2012, now abandoned.
(60) Provisional application No. 61/235,559, filed on Aug. 20, 2009.

(51) Int. Cl. A01K 67/027

(52) U.S. Cl. CPC A01K 67/0276; A01K 2207/05; A01K 2227/105; A01K 2217/075; A01K 2217/15; A01K 2267/0356

(57) ABSTRACT

This invention relates to the engineering of animal cells, preferably mammalian, more preferably rat, that are deficient due to the disruption of gene(s) or gene product(s) resulting in altered nervous system function. In one aspect, the altered function results in pain in the mammal. In another aspect, the nervous system dysfunction results in prolonged hyperalgesia, allodynia, and loss of sensory function. In another aspect, the invention relates to genetically modified rats, as well as the descendants and ancestors of such animals, which are animal models of altered nervous system function mediated pain and methods of their use. In another aspect, the genetically modified rats, as well as the descendants and ancestors of such animals, are animal models of nervous system dysfunction resulting in prolonged hyperalgesia, allodynia, and loss of sensory function and methods of their use. In another aspect, the present invention provides a method of identifying a compound useful for the treatment or prevention of pain.

### CROSS-REFERENCE TO RELATED APPLICATIONS

This application claims the benefit of U.S. Provisional Patent Application Ser. No. 61/235,559, filed Aug. 20, 2009, which application is hereby incorporated by reference in its entirety for all purposes.

### BACKGROUND OF THE INVENTION

Gene modification is a process whereby a specific gene, or a fragment of that gene, is altered. This alteration of the targeted gene may result in a change in the level of RNA and/or protein that is encoded by that gene, or the alteration may result in the targeted gene encoding a different RNA or protein than the untargeted gene. The modified gene may be studied in the context of a cell, or, more preferably, in the context of a genetically modified animal.

Genetically modified animals are among the most useful research tools in the biological sciences. An example of a genetically modified animal is a transgenic animal, which has a heterologous (i.e., foreign) gene, or gene fragment, incorporated into their genome that is passed on to their offspring. Although there are several methods of producing genetically modified animals, the most widely used is microinjection of DNA into single cell embryos. These embryos are then transferred into pseudopregnant recipient foster mothers. The offspring are then screened for the presence of the new gene, or gene fragment. Potential applications for genetically modified animals include discovering the genetic basis of human and animal diseases, generating disease resistance in humans and animals, gene therapy, toxicology studies, drug testing, pharmacokinetics and production of improved agricultural livestock.

Identification of novel genes and characterization of their function using mutagenesis has also been shown to be productive in identifying new drugs and drug targets. Creating in vitro cellular models that exhibit phenotypes that are clinically relevant provides a valuable substrate for drug target identification and screening for compounds that modulate not only the phenotype but also the target(s) that controls the phenotype. Modulation of such a target can provide information that validates the target as important for therapeutic intervention in a clinical disorder when such modulation of the target serves to modulate a clinically relevant phenotype.

Neuropathic pain is a chronic disease resulting from a dysfunction in the nervous system. This nervous system dysfunction often occurs due to peripheral nerve injury concentrated at the dorsal root ganglia (DRG), sensory neurons. Abnormal nervous function arises from injured axons, and from intact nociceptors that share receptivity with the injured nerve. The pathological conditions include prolonged hyperalgesia, allodynia, and loss of sensory function. Classical presentation of neuropathic pain within patients are: ubiquitous pain not otherwise explainable, sensory defect, burning pain, pain to light on the skin, sudden pain attacks without a clear provocation. Inflammation and traumatic nerve injury are major causes of nerve injuries. The genetic basis of such disorders derives from distorted connectivity, structure, and survival of neurons due to altered expression of genes.

Nociceptive pain is initiated by stimulation of nociceptors, and may be classified according to the mode of noxious stimulation; the most common categories being “thermal” (heat or cold), “mechanical” (crushing, tearing, etc.) and “chemical” (formalin, mustard oil, iodine in a cut, chili powder in the eyes).

Nociceptive pain may also be divided into “superficial somatic”, “deep”, “deep somatic” and “visceral”. Superficial somatic pain is initiated by activation of nociceptors in the skin or superficial tissues, and is sharp, well-defined and clearly located. Examples of injuries that produce superficial somatic pain include minor wounds and minor (first degree) burns. Deep somatic pain is initiated by stimulation of nociceptors in ligaments, tendons, bones, blood vessels, fasciae and muscles, and is dull, aching, poorly-localized pain; examples include sprains and broken bones. Visceral pain originates in the viscera (organs) and often is extremely difficult to locate, and several visceral regions produce “referred” pain when injured, where the sensation is located in an area distant from the site of injury or pathology

Psychogenic pain, also called psychalgia or somatoform pain, is pain caused, increased, or prolonged by mental, emotional, or behavioral factors. Headache or migraine, back pain, and stomach pain are sometimes diagnosed as psychogenic. Sufferers are often stigmatized, because both medical professionals and the general public tend to think that pain from a psychological source is not “real”. However, specialists consider that it is no less actual or hurtful than pain from any other source.

People with long term pain frequently display psychological disturbance, with elevated scores on the Minnesota Multiphasic Personality Inventory scales of hysteria, depression and hypochondriasis (the “neurotic triad”). Some investigators have argued that it is this neuroticism that causes acute injuries to turn chronic, but clinical evidence points the other way, to chronic pain causing neuroticism. When long term pain is relieved by therapeutic intervention, scores on the neurotic triad and anxiety fall, often to normal levels. Self-esteem, often low in chronic pain patients, also shows striking improvement once pain has resolved.

Central pain syndrome is a neurological condition caused by the malfunctioning of the Central Nervous System (CNS) which causes a sensitization of the pain system. The extent of pain and the areas affected are related to the cause of the injury, which can include trauma, tumors, stroke, Multiple Sclerosis, Parkinson's disease, or epilepsy. Pain can either be relegated to a specific part of the body or affect the body as a whole.

The discovery of relevant animal models for pain has led to a great advance in the study of this chronic disease. Animal models for pain can identify genes associated with pain by altered expression differences in pain related genes such as, transmitters, receptors, and ion channels. There are several wild type animal models which are induced in some fashion to model or exhibit altered pain response. One such model is the spared nerve injury (SNI) model. In this method surgery is done on animals under anesthesia to expose the sciatic nerve. The peroneal and tibial nerves are then ligated and sectioned. This model is especially useful because the responses to induced pain in this model reflect the clinical findings of patients with pain. Another pain model is the partial nerve injury (PNI) model. In the PNI model the sciatic nerve is partially injured via tight ligation such that the nerve is decreased in diameter around ½-⅓ the control. Another method to produce animal models which resemble pain is the spinal nerve ligation model (SNL). In this model both the L5 and L6 spinal nerves or the L5 alone are tightly ligated. This model resembles human pain as it presents long lasting hyperalgesia to noxious heat and mechanical allodynia, and spontanouse pain. Models have also been described to resemble human conditions of chronic pain caused not by trauma, but by disease states such as diabetic neuropathy. In these models diabetes can be induced in rats by injection with strptozotocin (STZ). The state of diabetes is measured by presence of hyperglycemia, or glucosuria. Other pain models include neuropathy due to drug side effects. One prime example is the anti-tumor agent paclitaxel which in humans produces sensory and peripheral neuropathy, mechanical allodynia, cold allodynia, chronic burning pain, and numbness or tingling. Many of these symptoms do not subside after treatment with the drug has been concluded. In one drug induced pain model rats were exposed to paclitaxel and vincristine and assessed for the presence of pain. After pain was assessed in drug induced models drug treatment studies to alleviate the induced pain serve as a great assay for pain treatments.

Once the pain model is induced the animals must be measured for exhibition of chronic pain. One method for pain measurement is mechano-cold sensitivity. In this detection method cold spray of different temperatures of extremity are applied to the hind paws of animals. The sensitivity to pain induction is evaluated by measuring the licking time and number of paw jerks. When an animal exhibits a pain phenotype which increases its sensitivity it will have a longer licking time and a larger number of jerks. Another cold behavioral test is to place a drop of acetone on the paw of an animal. The cold sensation given by the acetone is measured by observing response, usually within 20 seconds of acetone application. The response is recorded as paw withdraws, flicks or stamps, and licking or biting. Another method for analysis of pain is the paw withdraw threshold in response to probing with a form of pain induction. The pain induction is presented in a number of methods such as, electrical shock, heat or cold, probing with von Frey Filaments. Experimenters start out with the smallest diameter bristle (von Frey Filaments), they then establish a “baseline” response threshold by measuring at what force the wild-type rats will lift the paw. Then this threshold is studied with all animal models of induced pain. If the animal is more sensitive to the filament the animal is considered to be modeling a human in a or a chronic pain state. The animal models can then be tested for potential pain therapies to determine if the threshold has been altered in any way. Foot withdrawal latency due to radiant heat evocation has been shown to be a model of hyperalgesia. The animal is placed in a glass plate under which a light box is located which allows a small hole of light to be emitted on the heel or other position on the animal. The light is turned off after the animal has lifted its foot or adjusted due to response to the heat. The threshold of control temperature by which animals withdraw their feet is studied to identify increased or decreased sensitivity to induced pain by heat Animal models which display an altered expression in established or exploratory genes which may be involved in neuron connectivity, structure and survival are utilized in modeling pain. One method is to compare an animal model which has a full or partial deficiency in one or more genes with the control animal under scrutiny of induced pain. In this model the genetically altered animal is studied for hyer or hyposensitivity to pain inducing stimuli. In this fashion the animal model can be useful in discovering genes which may be involved in pain. The model can also be used for the discovery of drug targets. One example of altered gene expression in pain models is the transient receptor potential (TRP) channels. This family of non-selective cation channels is known to be important in sensory signaling in the peripheral nervous system. TRP channels have been characterized as temperature sensitive, and are highly expressed in the DRG nociceptors. The TRP channels are also implicated to have substantial response to inflammatory and traumatic nerve damage. Another pathway which affects peripheral axons and myelinating Schwann cells and may have a role in nervous system induced pain is neuregulin-1 (NRG1) and the erbB signaling pathway. Myelin is a product of Schwann cells and controls conduction velocity of vertebrate axons. NRG1 has been identified as a key mediator of axon-Schwann cell interactions and regulation of Schwann cell development. Due to its nerve pathology NRG1 and erbB signaling has gained attention as a major mediated of peripheral neuropathies and may be involved in allodynia and hyperalgesia. For these reasons rat models deficient for TRP channels, NRG1-erbB signaling pathways have been created and validate their role in pain as these models exhibit altered gene expression, and response to mechanical, cold, heat, disease, and drug induced pain.

Animal models exhibiting clinically relevant phenotypes are also valuable for drug discovery and development and for drug target identification. For example, mutation of somatic or germ cells facilitates the production of genetically modified offspring or cloned animals having a phenotype of interest. Such animals have a number of uses, for example as models of physiological disorders (e.g., of human genetic diseases) that are useful for screening the efficacy of candidate therapeutic compounds or compositions for treating or preventing such physiological disorders. Furthermore, identifying the gene(s) responsible for the phenotype provides potential drug targets for modulating the phenotype and, when the phenotype is clinically relevant, for therapeutic intervention. In addition, the manipulation of the genetic makeup of organisms and the identification of new genes have important uses in agriculture, for example in the development of new strains of animals and plants having higher nutritional value or increased resistance to environmental stresses (such as heat, drought, or pests) relative to their wild-type or non-mutant counterparts.

Since most eukaryotic cells are diploid, two copies of most genes are present in each cell. As a consequence, mutating both alleles to create a homozygous mutant animal is often required to produce a desired phenotype, since mutating one copy of a gene may not produce a sufficient change in the level of gene expression or activity of the gene product from that in the non-mutated or wild-type cell or multicellular organism, and since the remaining wild-type copy would still be expressed to produce functional gene product at sufficient levels. Thus, to create a desired change in the level of gene expression and/or function in a cell or multicellular organism, at least two mutations, one in each copy of the gene, are often required in the same cell.

In other instances, mutation in multiple different genes may be required to produce a desired phenotype. In some instances, a mutation in both copies of a single gene will not be sufficient to create the desired physiological effects on the cell or multi-cellular organism. However, a mutation in a second gene, even in only one copy of that second gene, can reduce gene expression levels of the second gene to produce a cumulative phenotypic effect in combination with the first mutation, especially if the second gene is in the same general biological pathway as the first gene. This effect can alter the function of a cell or multi-cellular organism. A hypomorphic mutation in either gene alone could result in protein levels that are severely reduced but with no overt effect on physiology. Severe reductions in the level of expression of both genes, however, can have a major impact. This principle can be extended to other instances where mutations in multiple (two, three, four, or more, for example) genes are required cumulatively to produce an effect on activity of a gene product or on another phenotype in a cell or multi-cellular organism. It should be noted that, in this instance, such genes may all be expressed in the same cell type and therefore, all of the required mutations occur in the same cell. However, the genes may normally be expressed in different cell types (for example, secreting the different gene products from the different cells). In this case, the gene products are expressed in different cells but still have a biochemical relationship such that one or more mutations in each gene is required to produce the desired phenotype.

### BRIEF SUMMARY OF THE INVENTION

In accordance with the purposes of this invention, as embodied and broadly described herein, this invention relates to the engineering of animal cells, preferably mammalian, more preferably rat, that are deficient due to the disruption of gene(s) or gene product(s) resulting in altered pain gene expression, pain sensation or any pain phenotype.

In another aspect, the invention relates to genetically modified rats, as well as the descendants and ancestors of such animals, which are animal models of human pain and methods of their use.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWING

This invention, as defined in the claims, can be better understood with reference to the following drawings:

FIGS. 1-4 show the process for creating a genetically modified pain rat model using DNA transposons to create an insertion mutation directly in the germ line.

FIG. 1: Gene modification by DNA transposons.

FIG. 2: Breeding strategy for creating rat knockouts directly in the germ cells with DNA transposons.

FIG. 3: DNA sequences

FIG. 4: DNA transposon-mediated insertion mutation in Rattus norvegicus Trpc4 gene.

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

### Definitions

The present invention may be understood more readily by reference to the following detailed description of preferred embodiments of the invention and the Examples included therein and to the Figures and their previous and following description. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, reference is made to various proteins and nucleic acids. It is understood that any names used for proteins or nucleic acids are art-recognized names, such that the reference to the name constitutes a disclosure of the molecule itself.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

“Complementary,” as used herein, refers to the subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

A “deletion mutation” means a type of mutation that involves the loss of genetic material, which may be from a single base to an entire piece of chromosome. Deletion of one or more nucleotides in the DNA could alter the reading frame of the gene; hence, it could result in a synthesis of a nonfunctional protein due to the incorrect sequence of amino acids during translation.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed”. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene”, also called a “structural gene” means a DNA sequence that codes for or corresponds to a particular sequence of amino acids which comprise all or part of one or more proteins or enzymes, and may or may not include introns and regulatory DNA sequences, such as promoter sequences, 5′-untranslated region, or 3′-untranslated region which affect for example the conditions under which the gene is expressed. Some genes, which are not structural genes, may be transcribed from DNA to RNA, but are not translated into an amino acid sequence. Other genes may function as regulators of structural genes or as regulators of DNA transcription.

By “genetically modified” is meant a gene that is altered from its native state (e.g. by insertion mutation, deletion mutation, nucleic acid sequence mutation, or other mutation), or that a gene product is altered from its natural state (e.g. by delivery of a transgene that works in trans on a gene's encoded mRNA or protein, such as delivery of inhibitory RNA or delivery of a dominant negative transgene).

By “exon” is meant a region of a gene which includes sequences which are used to encode the amino acid sequence of the gene product.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature.

As used herein, the term “homology” refers to the subunit sequence identity or similarity between two polymeric molecules e.g., between two nucleic acid molecules, e.g., between two DNA molecules, or two polypeptide molecules. When a subunit position in both of the two molecules is occupied by the same monomeric subunit, e.g., if a position in each of two polypeptide molecules is occupied by phenylalanine, then they are identical at that position. The homology between two sequences, most clearly defined as the % identity, is a direct function of the number of identical positions, e.g., if half (e.g., 5 positions in a polymer 10 subunits in length) of the positions in two polypeptide sequences are identical then the two sequences are 50% identical; if 70% of the positions, e.g., 7 out of 10, are matched or homologous, the two sequences share 70% identity. By way of example, the polypeptide sequences ACDEFG and ACDHIK share 50% identity and the nucleotide sequences CAATCG and CAAGAC share 50% identity.

“Homologous recombination” is the physical exchange of DNA expedited by the breakage and reunion of two non-sister chromatids. In order to undergo recombination the DNA duplexes must have complimentarity. The molecular mechanism is as follows: DNA duplexes pair, homologous strands are nicked, and broken strands exchange DNA between duplexes. The region at the site of recombination is called the hybrid DNA or heteroduplex DNA. Second nicks are made in the other strand, and the second strand crosses over between duplexes. After this second crossover event the reciprocal recombinant or splice recombinant is created. The duplex of one DNA parent is covalently linked to the duplex of another DNA parent. Homologous recombination creates a stretch of heteroduplex DNA.

A “hypomorphic mutation” is a change to the genetic material (usually DNA or RNA), which can be caused by any form of genetic mutation, and causes an decrease in normal gene function without causing a complete absence of normal gene function.

The term “inbred animal” is used herein to refer to an animal that has been interbred with other similar animals of the same species in order to preserve and fix certain characteristics, or to prevent other characteristics from being introduced into the breeding population.

The term “insertional mutation” is used herein to refer the translocation of nucleic acid from one location to another location which is in the genome of an animal so that it is integrated into the genome, thereby creating a mutation in the genome. Insertional mutations can also include knocking out or knocking in of endogenous or exogenous DNA via gene trap or cassette insertion. Exogenous DNA can access the cell via electroporation or chemical transformation. If the exogenous DNA has homology with chromosomal DNA it will align itself with endogenous DNA. The exogenous DNA is then inserted or disrupts the endogenous DNA via two adjacent crossing over events, known as homologous recombination. A targeting vector can use homologous recombination for insertional mutagenesis. Insertional mutagenesis of endogenous or exogenous DNA can also be carried out via DNA transposon. The DNA transposon is a mobile element that can insert itself along with additional exogenous DNA into the genome. Insertional mutagenesis of endogenous or exogenous DNA can be carried out by retroviruses. Retroviruses have a RNA viral genome that is converted into DNA by reverse transcriptase in the cytoplasm of the infected cell. Linear retroviral DNA is transported into the nucleus, and become integrated by an enzyme called integrase. Insertional mutagenesis of endogenous or exogenous DNA can also be done by retrotransposons in which an RNA intermediate is translated into DNA by reverse transcriptase, and then inserted into the genome.

The term “gene knockdown” refers to techniques by which the expression of one or more genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knockdown organism” or “knockdowns”.

By “knock-out” is meant an alteration in the nucleic acid sequence that reduces the biological activity of the polypeptide normally encoded therefrom by at least 80% compared to the unaltered gene. The alteration may be an insertion, deletion, frameshift mutation, or missense mutation. Preferably, the alteration is an insertion or deletion, or is a frameshift mutation that creates a stop codon.

An “L1 sequence” or “L1 insertion sequence” as used herein, refers to a sequence of DNA comprising an L1 element comprising a 5′ UTR, ORF1 and ORF2, a 3′ UTR and a poly A signal, wherein the 3′ UTR has DNA (e.g. a gene trap or other cassette) positioned either therein or positioned between the 3′ UTR and the poly A signal, which DNA is to be inserted into the genome of a cell.

A “mutation” is a detectable change in the genetic material in the animal, which is transmitted to the animal's progeny. A mutation is usually a change in one or more deoxyribonucleotides, the modification being obtained by, for example, adding, deleting, inverting, or substituting nucleotides. Exemplary mutations include but are not limited to a deletion mutation, an insertion mutation, a nonsense mutation or a missense mutation. Thus, the terms “mutation” or “mutated” as used herein are intended to denote an alteration in the “normal” or “wild-type” nucleotide sequence of any nucleotide sequence or region of the allele. As used herein, the terms “normal” and “wild-type” are intended to be synonymous, and to denote any nucleotide sequence typically found in nature. The terms “mutated” and “normal” are thus defined relative to one another; where a cell has two chromosomal alleles of a gene that differ in nucleotide sequence, at least one of these alleles is a “mutant” allele as that term is used herein. Based on these definitions, an “endogenous toxicology gene” is the “wild-type” gene that exists normally in a cell, and a “mutated toxicology gene” defines a gene that differs in nucleotide sequence from the wild-type gene.

“Non-homologous end joining (NHEJ)” is a cellular repair mechanism. The NHEJ pathway is defined by the ligation of blunt ended double stand DNA breaks. The pathway is initiated by double strand breaks in the DNA, and works through the ligation of DNA duplex blunt ends. The first step is recognition of double strand breaks and formation of scaffold. The trimming, filling in of single stranded overhangs to create blunt ends and joining is executed by the NHEJ pathway. An example of NHEJ is repair of a DNA cleavage site created by a zinc finger nuclease (ZFN). This would normally be expected to create a small deletion mutation.

“Nucleic Acid sequence mutation” is a mutation to the DNA of a gene that involves change of one or multiple nucleotides. A point mutation which affects a single nucleotide can result in a transition (purine to purine or pyrimidine to pyrimidine) or a transversion (purine to pyrimidine or pyrimidine to purine). A point mutation that changes a codon to represent a different amino acid is a missense mutation. Some point mutations can cause a change in amino acid so that there is a premature stop codon; these mutations are called nonsense mutations. A mutation that inserts or deletes a single base will change the entire downstream sequence and are known as frameshift mutations. Some mutations change a base pair but have no effect on amino acid representation; these are called silent mutations. Mutations to the nucleic acid of a gene can have different consequences based on their location (intron, exon, regulatory sequence, and splice joint).

“Optional” or “optionally” means that the subsequently described event or circumstance may or may not occur, and that the description includes instances where said event or circumstance occurs and instances where it does not.

The term “outbred animal” is used herein to refer to an animal that breeds with any other animal of the same species without regard to the preservation of certain characteristics.

As used herein, the term “phenotype” means any property of a cell or organism. A phenotype can simply be a change in expression of an mRNA or protein. Examples of phenotypes also include, but are in no way limited to, cellular, biochemical, histological, behavioral, or whole organismal properties that can be detected by the artisan. Phenotypes include, but are not limited to, cellular transformation, cell migration, cell morphology, cell activation, resistance or sensitivity to drugs or chemicals, resistance or sensitivity to pathogenic protein localization within the cell (e.g. translocation of a protein from the cytoplasm to the nucleus), resistance or sensitivity to ionizing radiation, profile of secreted or cell surface proteins, (e.g., bacterial or viral) infection, post-translational modifications, protein localization within the cell (e.g. translocation of a protein from the cytoplasm to the nucleus), profile of secreted or cell surface proteins, cell proliferation, signal transduction, metabolic defects or enhancements, transcriptional activity, recombination intermediate joining, DNA damage response, cell or organ transcript profiles (e.g., as detected using gene chips), apoptosis resistance or sensitivity, animal behavior, organ histology, blood chemistry, biochemical activities, gross morphological properties, life span, tumor susceptibility, weight, height/length, immune function, organ function, any disease state, and other properties known in the art. In certain situations and therefore in certain embodiments of the invention, the effects of mutation of one or more genes in a cell or organism can be determined by observing a change in one or more given phenotypes (e.g., in one or more given structural or functional features such as one or more of the phenotypes indicated above) of the mutated cell or organism compared to the same structural or functional feature(s) in a corresponding wild-type or (non-mutated) cell or organism (e.g., a cell or organism in which the gene(s) have not been mutated).

By “plasmid” is meant a circular strand of nucleic acid capable of autosomal replication in plasmid-carrying bacteria. The term includes nucleic acid which may be either DNA or RNA and may be single- or double-stranded. The plasmid of the definition may also include the sequences which correspond to a bacterial origin of replication.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease Si), as well as protein binding domains (consensus sequences) responsible for the binding of RNA polymerase. The promoter may be operatively associated with other expression control sequences, including enhancer and repressor sequences.

A “random site” is used herein to refer to a location in the genome where a retrotransposition or transposition or other DNA mutation event takes places, without prior intention of mutation at that particular location. It is also used herein to refer to a location in the genome that is randomly modified by any insertion mutation or deletion mutation or nucleic acid sequence mutation.

The term “regulatory sequence” is defined herein as including promoters, enhancers and other expression control elements such as polyadenylation sequences, matrix attachment sites, insulator regions for expression of multiple genes on a single construct, ribosome entry/attachment sites, introns that are able to enhance expression, and silencers.

By “reporter gene” is meant any gene which encodes a product whose expression is detectable. A reporter gene product may have one of the following attributes, without restriction: fluorescence (e.g., green fluorescent protein), enzymatic activity (e.g., lacZ or luciferase), or an ability to be specifically bound by a second molecule (e.g., biotin or an antibody-recognizable epitope).

By “retrotransposition” as used herein, is meant the process of integration of a sequence into a genome, expression of that sequence in the genome, reverse transcription of the integrated sequence to generate an extrachromosomal copy of the sequence and reintegration of the sequence into the genome.

A “retrotransposition event” is used herein to refer to the translocation of a retrotransposon from a first location to a second location with the preferable outcome being integration of a retrotransposon into the genome at the second location. The process involves a RNA intermediate, and can retrotransposc from one chromosomal location to another or from introduced exogenous DNA to endogenous chromosomal DNA.

By “selectable marker” is meant a gene product which may be selected for or against using chemical compounds, especially drugs. Selectable markers often are enzymes with an ability to metabolize the toxic drugs into non-lethal products. For example, the pac (puromycin acetyl transferase) gene product can metabolize puromycin, the dhfr gene product can metabolize trimethoprim (tmp) and the Ma gene product can metabolize ampicillin (amp). Selectable markers may convert a benign drug into a toxin. For example, the HSV tk gene product can change its substrate, FIAU, into a lethal substance. Another selectable marker is one which may be utilized in both prokaryotic and eukaryotic cells. The neo gene, for example, metabolizes and neutralizes the toxic effects of the prokaryotic drug, kanamycin, as well as the eukaryotic drug, G418.

By “selectable marker gene” as used herein is meant a gene or other expression cassette which encodes a protein which facilitates identification of cells into which the selectable marker gene is inserted.

A “specific site” is used herein to refer to a location in the genome that is predetermined as the position where a retrotransposition or transposition event or other DNA mutation will take place. It is also used herein to refer to a specific location in the genome that is modified by any insertion mutation or deletion mutation or nucleic acid sequence mutation.

A “pain gene” is used herein to refer to a gene which encodes a protein that is associated with the phenotype that is characterized as altering the expression and functionality of signaling or pathways involved in pain. The functions of pain genes may produce phenotypes in all types of pain including but not limited to neuropathic, nociceptive, somatic, visceral, central, and psychogenic pain including migraine. Gene expression can effect but it not limited to myelin conduction, Schwann cell development and function, neuron-glia interactions, transmitters, receptors, ion channels, sensory signaling, temperator sensitivity, mechanical stimulation, disease state (e.g. diabetes) neuropathy, inflammatory or traumatic nerve injury. This phenotype may affect the activity, localization, interactions of neuropathic, nociceptive, visceral, central and peripheral signaling, or any other interaction which the substance may have within humans, rats and other model organisms. A “pain protein” is used herein to refer to a protein product of a gene that is associated with the nerve response phenotype that is characterized as altering the response to induced pain, chronic or spontaneous pain, sensory defect, burning pain, light stroking pain, sudden pain attacks.

As used herein, the term “targeted genetic recombination” refers to a process wherein recombination occurs within a DNA target locus present in a host cell or host organism. Recombination can involve either homologous or non-homologous DNA.

The term “transfection” means the introduction of a foreign nucleic acid into a cell. The term “transformation” means the introduction of a “foreign” (i.e. extrinsic or extracellular) gene, DNA or RNA sequence to an ES cell or pronucleus, so that the cell will express the introduced gene or sequence to produce a desired substance in a genetically modified animal.

By “transgenic” is meant any animal which includes a nucleic acid sequence which is inserted by artifice into a cell and becomes a part of the genome of the animal that develops from that cell. Such a transgene may be partly or entirely heterologous to the transgenic animal. Although transgenic mice represent another embodiment of the invention, other transgenic mammals including, without limitation, transgenic rodents (for example, hamsters, guinea pigs, rabbits, and rats), and transgenic pigs, cattle, sheep, and goats are included in the definition.

By “transposition” as used herein, is meant the process of one DNA sequence insertion into another (location) without relying on sequence homology. The DNA element can be transposed from one chromosomal location to another or from introduction of exogenous DNA and inserted into the genome.

A “transposition event” or “transposon insertion sequence” is used herein to refer to the translocation of a DNA transposon either from one location on the chromosomal DNA to another or from one location on introduced exogenous DNA to another on the chromosomal DNA.

By “transposon” or “transposable element” is meant a linear strand of DNA capable of integrating into a second strand of DNA which may be linear or may be a circularized plasmid. Transposons often have target site duplications, or remnants thereof, at their extremities, and are able to integrate into similar DNA sites selected at random, or nearly random. Preferred transposons have a short (e.g., less than 300) base pair repeat at either end of the linear DNA. By “transposable elements” is meant any genetic construct including but not limited to any gene, gene fragment, or nucleic acid that can be integrated into a target DNA sequence under control of an integrating enzyme, often called a transposase.

A coding sequence is “under the control of” or “operatively associated with” transcriptional and translational control sequences in a cell when RNA polymerase transcribes the coding sequence into mRNA, which is then trans-RNA spliced (if it contains introns) and translated, in the case of mRNA, into the protein encoded by the coding sequence.

The term “variant” may also be used to indicate a modified or altered gene, DNA sequence, enzyme, cell, etc., i.e., any kind of mutant.

The term “vector” is used interchangeably with the terms “construct”, “cloning vector” and “expression vector” and means the vehicle by which a DNA or RNA sequence (e.g. a foreign gene) can be introduced into a host cell, (e.g. ES cell or pronucleus) so as to transform the host and promote expression (e.g. transcription and translation) of the introduced sequence including but not limited to plasmid, phage, transposons, retrotransposons, viral vector, and retroviral vector. By “non-viral vector” is meant any vector that does not comprise a virus or retrovirus.

A “vector sequence” as used herein, refers to a sequence of DNA comprising at least one origin of DNA replication and at least one selectable marker gene.

For the purposes of the present invention, the term “zinc finger nuclease” or “ZFN” refers to a chimeric protein molecule comprising at least one zinc finger DNA binding domain effectively linked to at least one nuclease or part of a nuclease capable of cleaving DNA when fully assembled. Ordinarily, cleavage by a ZFN at a target locus results in a double stranded break (DSB) at that locus.

The present invention provides a desired rat or a rat cell which contains a predefined, specific and desired alteration rendering the rat or rat cell predisposed to abnormal perception of pain by modification of its structure or mechanism. Specifically, the invention pertains to a genetically altered rat, or a rat cell in culture, that is defective in at least one of two alleles of a neuropathic pain gene such as the Nrg1, Trpc4, ErbB4 gene, the Cyp3a4 gene, etc. In one embodiment, the neuropathic pain gene is the Nrg1, Trpc4, ErbB4 gene. In another embodiment, the pain gene is one or more pain genes, selected from the group consisting of Cyp3a4, Nrg1 NC_005115.2, Trpc4 NC_005101.2, Trpv1 NC_005109.2, Trpv3 NC_005109.2, ErbB4 NC_005108.2, Pparα NC_005106.2, Pparγ NC_005103.2, Trpml3 (NA), Trpml6 (NA), Trpm8 NC_005108.2, Trpv1 NC_005109.2, Trpa1 NC_005104.2, Trpc3 NC_005101.2, Trpc5 NC_005120.2, Scn9a NC_005102.2, Ntrk1 NC_005101.2, Wnk1 NC_005103.2, Hsan1 (NA), Sc10a (NA), Hsan3 (NA), Ptger2 NC_005114.2, Pnoc NC_005114.2, Gabbr1 NC_005119.2, Gabbr2 NC_005104.2, Cacna1g NC_005109.2, Tac1 NC_005103.2, Prx NC_005100.2, Homer1 (NA), Scn11a NC_005107.2, Oprl1 NC_005102.2, Prlhr NC_005100.2, P2x3 NC_005102.2, Bdkrb1 NC_005105.2, Ptgs2 NC_000001.10, Th NC_005100.2, Npy1r NC_005115.2, P2rx4 NC_005111.2, Mmp9 NC_005102.2, Mmp2 NC_005118.2, and Bdnf.

The inactivation of at least one of these pain alleles results in an animal with an altered pain response. In one embodiment, the genetically altered animal is a rat of this type and is able to serve as a useful model for pain induced by nerve alteration, disease state, drug treatment or spontaneously. The invention additionally pertains to the use of such rats or rat cells, and their progeny in research and medicine.

In one embodiment, the invention provides a genetically modified or chimeric rat cell whose genome comprises two chromosomal alleles of a pain gene (especially, the Nrg1, Trpc4, ErbB4 gene), wherein at least one of the two alleles contains a mutation, or the progeny of this cell. The invention includes the embodiment of the above animal cell, wherein one of the alleles expresses a normal pain gene product. The invention includes the embodiment wherein the rat cell is a pluripotent cell such as an embryonic cell, embryonic stein (ES) cell, induced pluripotent stem cell (iPS), or spermatagonial stem (SS) cell, and in particular, wherein the pain gene is the gene. In another embodiment, the pain gene is one or more pain genes, selected from the group consisting of Cyp3a4, Nrg1 NC_005115.2, Trpc4 NC_005101.2, Trpv1 NC_005109.2, Trpv3 NC_005109.2, ErbB4 NC_005108.2, Pparα NC_005106.2, Pparγ NC_005103.2, Trpml3 (NA), Trpml6 (NA), Trpm8 NC_005108.2, Trpv1 NC_005109.2, Trpa1 NC_005104.2, Trpc3 NC_005101.2, Trpc5 NC_005120.2, Scn9a NC_005102.2, Ntrk1 NC_005101.2, Wnk1 NC_005103.2, Hsan1 (NA), Sc10a (NA), Hsan3 (NA), Ptger2 NC_005114.2, Pnoc NC_005114.2, Gabbr1 NC_005119.2, Gabbr2 NC_005104.2, Cacna1g NC_005109.2, Tac1 NC_005103.2, Prx NC_005100.2, Homer1 (NA), Scn11a NC_005107.2, Oprl1 NC_005102.2, Prlhr NC_005100.2, P2x3 NC_005102.2, Bdkrb1 NC_005105.2, Ptgs2 NC_000001.10, Th NC_005100.2, Npy1r NC_005115.2, P2rx4 NC_005111.2, Mmp9 NC_005102.2, Mmp2 NC_005118.2, and Bdnf. In another embodiment, the rat cell is a somatic cell.

The methods of the present invention can be used to mutate any eukaryotic cell, including, but not limited to, haploid (in the case of multiple gene mutations), diploid, triploid, tetraploid, or aneuploid. In one embodiment, the cell is diploid. Cells in which the methods of the present invention can be advantageously used include, but are not limited to, primary cells (e.g., cells that have been explanted directly from a donor organism) or secondary cells (e.g., primary cells that have been grown and that have divided for some period of time in vitro, e.g., for 10-100 generations). Such primary or secondary cells can be derived from multi-cellular organisms, or single-celled organisms. The cells used in accordance with the invention include normal cells, terminally differentiated cells, or immortalized cells (including cell lines, which can be normal, established or transformed), and can be differentiated (e.g., somatic cells or germ cells) or undifferentiated (e.g., multipotent, pluripotent or totipotent stem cells).

A variety of cells isolated from the above-referenced tissues, or obtained from other sources (e.g., commercial sources or cell banks), can be used in accordance with the invention. Non-limiting examples of such cells include somatic cells such as immune cells (T-cells, B-cells, Natural Killer (NK) cells), blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, thymic nurse cells, Schwann cells, etc.). Eukaryotic germ cells (spermatocytes and oocytes) can also be used in accordance with the invention, as can the progenitors, precursors and stem cells that give rise to the above-described somatic and germ cells. These cells, tissues and organs can be normal, or they can be pathological such as those involved in diseases or physical disorders, including but not limited to immune related diseases, chronic inflammation, autoimmune responses, infectious diseases (caused by bacteria, fungi or yeast, viruses (including HIV) or parasites), in genetic or biochemical pathologies (e.g., cystic fibrosis, hemophilia, Alzheimer's disease, schizophrenia, muscular dystrophy, multiple sclerosis, etc.), or in carcinogenesis and other cancer-related processes. Rat pluripotent cells, including embryonic cells, spermatogonial stem cells, embryonic stem cells, and iPS cells are envisioned. Rat somatic cells are also envisioned.

In certain embodiments of the invention, cells can be mutated within the organism or within the native environment as in tissue explants (e.g., in vivo or in situ). Alternatively, tissues or cells isolated from the organism using art-known methods and genes can be mutated according to the present methods. The tissues or cells are either maintained in culture (e.g., in vitro), or re-implanted into a tissue or organism (e.g., ex vivo).

The invention also includes a non-human genetically modified or chimeric rat whose genome comprises two chromosomal alleles of a pain gene, wherein at least one of the two alleles contains a mutation, or the progeny of the animal, or an ancestor of the animal, at an embryonic stage (preferably the one-cell, or fertilized oocyte stage, and generally, not later than about the 8-cell stage) contains a mutation. The invention also includes the embodiment wherein the pain gene of the rat is the Nrg1, Trpc4, ErbB4 gene. In another embodiment, the pain gene is one of several known pain genes, such as In another embodiment, the pain gene is one or more pain genes, selected from the group consisting of Cyp3a4, Nrg1 NC_005115.2, Trpc4 NC_005101.2, Trpv1 NC_005109.2, Trpv3 NC_005109.2, ErbB4 NC_005108.2, Pparα NC_005106.2, Pparγ NC_005103.2, Trpml3 (NA), Trpml6 (NA), Trpm8 NC_005108.2, Trpv1 NC_005109.2, Trpa1 NC_005104.2, Trpc3 NC_005101.2, Trpc5 NC_005120.2, Scn9a NC_005102.2, Ntrk1 NC_005101.2, Wnk1 NC_005103.2, Hsan1 (NA), Sc10a (NA), Hsan3 (NA), Ptger2 NC_005114.2, Pnoc NC_005114.2, Gabbr1 NC_005119.2, Gabbr2 NC_005104.2, Cacna1g NC_005109.2, Tac1 NC_005103.2, Prx NC_005100.2, Homer1 (NA), Scn11a NC_005107.2, Oprl1 NC_005102.2, Prlhr NC_005100.2, P2x3 NC_005102.2, Bdkrb1 NC_005105.2, Ptgs2 NC_000001.10, Th NC_005100.2, Npy1r NC_005115.2, P2rx4 NC_005111.2, Mmp9 NC_005102.2, Mmp2 NC_005118.2, and Bdnf. The invention is also directed to the embodiment wherein the animal cell is a rat pluripotent cell. The invention is also directed to the embodiment wherein the animal cell is a rat somatic cell.

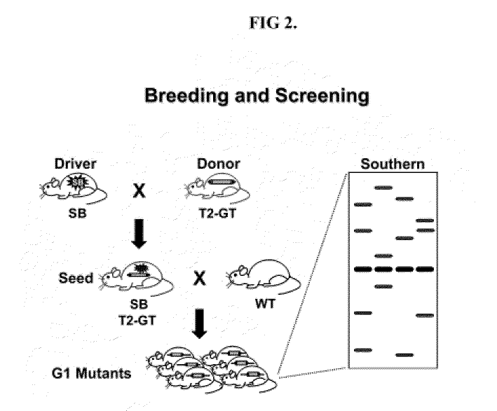

In one embodiment, the pain gene is mutated directly in the germ cells of a living organism. The separate transgenes for DNA transposon flanking ends and transposase are facilitated to create an active DNA transposon which integrates into the rat's genome. A plasmid containing tranposon inverted repeats is used to create the transgenic “donor” rat. A plasmid containing transposase is used to create a separate transgenic “driver” rat. The donor rat is then bred with the driver rat to produce a rat which contains both donor transposon with flanking repeats and driver transposase (FIG. 2). This rat known as the “seed” rat has an activated DNA transposase which drives transposition events. The seed rat is bred to wild type rats to create heterozygote progeny with new transposon insertions. The heterozygotes can be interbred to create homozygous rats. Transposon insertion mutations are identified and recovered via a cloning and sequencing strategy involving the transposon-cellular DNA junction fragments. The rats that are identified to have a new DNA transposon insertion in a known gene or EST or DNA sequence of interest are called knockout rats.

In one embodiment, the pain gene is mutated in the oocyte before fusion of the pronuclei. This method for genetic modification of rats uses microinjected DNA into the male pronucleus before nuclear fusion. The microinjected DNA creates a genetically modified founder rat. A female rat is mated and the fertilized eggs are flushed from their oviducts. After entry of the sperm into the egg, the male and female pronuclei are separate entities until nuclear fusion occurs. The male pronucleus is larger are can be identified via dissecting microscope. The egg can be held in place by micromanipulation using a holding pipette. The male pronucleus is then microinjected with DNA that can be genetically modified. The microinjected eggs are then implanted into a surrogate pseudopregnant female which was mated with a vasectomized male for uterus preparation. The foster mother gives birth to genetically modified animal. The microinjection method can introduce genetic modifications directly to the germline of a living animal.

In another embodiment, the pain gene is mutated in a pluripotent cell. These pluripotent cells can proliferate in cell culture and be genetically modified without affecting their ability to differentiate into other cell types including germ line cells. Genetically modified pluripotent cells from a donor can be microinjected into a recipient blastocyst, or in the case of spermatogonial stem cells can be injected into the rete testis of a recipient animal. Recipient genetically modified blastocysts are implated into pseudopregnant surrogate females. The progeny which have a genetic modification to the germline can then be established, and lines homozygous for the genetic modification can be produced by interbreeding.

In another embodiment, the pain gene is mutated in a somatic cell and then used to create a genetically modified animal by somatic cell nuclear transfer. Somatic cell nuclear transfer uses embryonic, fetal, or adult donor cells which are isolated, cultured, and/or modified to establish a cell line. Individual donor cells are fused to an enucleated oocyte. The fused cells are cultured to blastocyst stage, and then transplanted into the uterus of a pseudopregnant female.

In one embodiment, the present invention is directed to methods for mutating a single gene or multiple genes (e.g., two or more) in eukaryotic cells and multicellular organisms. The present invention contemplates several methods for creating mutations in the pain gene(s). In one embodiment the mutation is an insertion mutation. In another embodiment the mutation is a deletion mutation. In another embodiment the method of mutation is the introduction of a cassette or gene trap by recombination. In another embodiment a small nucleic acid sequence change is created by mutagenesis (through the creation of frame shifts, stop mutations, substitution mutations, small insertion mutations, small deletion mutations, and the like). In yet another embodiment, a transgene is delivered to knockout or knockdown the products of the pain gene (mRNA or protein) in trans.

The invention also is directed to insertional mutagens for making the mutant cells and organisms, and which also can be used to analyze the mutations that are made in the cells and organisms. The invention also is directed to methods in which one or more mutated genes is tagged by a tag provided by the insertional mutagen to allow the detection, selection, isolation, and manipulation of a cell with a genome tagged by the insertional mutagen and allows the identification and isolation of the mutated gene(s). The invention provides methods for making multiple mutations (i.e., mutations in two or more genes that produce a phenotype cumulatively) in cells and organisms and tagging at least one of the mutated genes such that the mutation can be rapidly identified.

The term gene disruption as used herein refers to a gene knock-out or knock-down in which an insertional mutagen is integrated into an endogenous gene thereby resulting expression of a fusion transcript between endogenous exons and sequences in the insertional mutagen.

In one embodiment, the invention provides for insertional mutagenesis involving the integration of one or more polynucleotide sequences into the genome of a cell or organism to mutate one or more endogenous genes in the cell or organism. Thus, the insertional mutagenic polynucleotides of the present invention are designed to mutate one or more endogenous genes when the polynucleotides integrate into the genome of the cell.

Accordingly, the insertional mutagens used in the present invention can comprise any nucleotide sequence capable of altering gene expression levels or activity of a gene product upon insertion into DNA that contains the gene. The insertional mutagens can be any polynucleotide, including DNA and RNA, or hybrids of DNA and RNA, and can be single-stranded or double-stranded, naturally occurring or non-naturally occurring (e.g., phosphorothioate, peptide-nucleic acids, etc.). The insertional mutagens can be of any geometry, including but not limited to linear, circular, coiled, supercoiled, branched, hairpin, and the like, and can be any length capable of facilitating mutation, and tagging of an endogenous gene. In certain embodiments, the insertional mutagens can comprise one or more nucleotide sequences that provide a desired function.

In another embodiment, the method further involves transforming a cell with a nucleic acid construct comprising donor DNA. An example of donor DNA may include a DNA transposon. Transposable elements are discrete sequences in the genome which are mobile. They have the ability to translocate from one position in the genome to another. Unlike most genetic entities that can create modification to an organism's genome, transposons do not require homology with the recipient genome for insertion. Transposons contain inverted terminal repeats which are recognized by the protein transposase. Transposase facilitates the transposition event. Transposition can occur in replicative (the element is duplicated) or nonreplicative (element moves from one site to another and is conserved) mechanism. Transposons can either contain their own transposase or transposase can be added in trans to facilitate transposition. The transposon promotes genetic modifications in many ways. The insertion itself may cause genetic modification by disruption of a DNA sequence or introduction of DNA. The transposon may be used to deliver a gene trap.

In another embodiment, the method for mutagenesis involves transforming a cell with nucleic acid by use of a LTR retrotransposon with reverse transcriptase. The retrotransposon is initially composed of a single strand of RNA. This single stranded RNA is converted into a double stranded DNA by reverse transcriptase. This is a linear duplex of DNA that is integrated into the host's genome by the enzyme integrase. This insertion event is much like a transposition event and can be engineered to genetically modify a host's genome.

In another embodiment, the method for mutageneis is a non-LTR retrotransposon. Long Interspersed Nucleotide Elements (LINEs) are retrotransposons that do not have long terminal repeats (LTR's). The LINES open reading frame 1 (ORF1) is a DNA binding protein, ORF2 provides both reverse transcriptase and endonuclease activity. The endonucleolytic nick provides the 3′-OH end required for priming the synthesis of cDNA on the RNA template by reverse transcriptase. A second cleavage site opens the other strand of DNA. The RNA/DNA hybrid integrates into the host genome before or after converting into double stranded DNA. The integration process is called target primed reverse transcription (TPRT).

In another embodiment a retrovirus may be used for insertional genetic modification. The retroviral vector (e.g. lentivirus) inserts itself into the genome. The vector can carry a transgene or can be used for insertional mutagenesis. The infected embryos are then injected into a receptive female. The female gives birth to founder animals which have genetic modifications in their germline. Genetically modified lines are established with these founder animals.

In another embodiment, mutagenesis by recombination of a cassette into the genome may be facilitated by targeting constructs or homologous recombination vectors. Homologous recombination vectors are composed of fragments of DNA which are homologous to target DNA. Recombination between identical sequences in the vector and chromosomal DNA will result in genetic modification. The vector may also contain a selection method (e.g., antibiotic resistance or GFP) and a unique restriction enzyme site used for further genetic modification. The targeting vector will insert into the genome at a position (e.g, exon, intron, regulatory element) and create genetic modification.

In another embodiment, mutagenesis through recombination of a cassette into the genome may be carried out by Serine and Tyrosine recombinase with the addition of an insertion cassette. Site-specific recombination occurs by recombinase protein recognition of DNA, cleavage and rejoining as a phosphodiesterase bond between the serinc or tyrosine residues. A cassette of exogenous or endogenous DNA may be recombined into the serine or tyrosine site. The cassette can contain a transgene, gene trap, reporter gene or other exogenous or endogenous DNA.

In one embodiment, the present invention is directed to methods for both targeted (site-specific) DNA insertions and targeted DNA deletions. In one embodiment, the method involves transformation of a cell with a nucleic acid or mRNA construct minimally comprising DNA encoding a chimeric zinc finger nuclease (ZFN), which can be used to create a DNA deletion. In another embodiment, a second DNA construct can be provided that will serve as a template for repair of the cleavage site by homologous recombination. In this embodiment, a DNA insertion may be created. The DNA insertion may contain a gene trap cassette.

The invention also is directed to nucleic acid sequence mutation for making the mutant cells and organisms.

In one embodiment, the method involves chemical mutagenesis with mutagens such as methane-sulfonic acid ethylester (EMS), N-ethyl-N-nitrosourea (ENU), diepoxyoctane and UV/trimethylpsorlalen to create nucleic acid sequence mutations.

In another embodiment, sequence editing methods are used that involve the delivery of small DNA fragments, hybrid DNA/RNA molecules, and modified DNA polymers to create sequence mismatches and nucleic acid mutations. RNA/DNA hybrids are molecules composed of a central stretch of DNA flanked by short RNA sequences that form hairpin structures. The RNA/DNA hybrids can produce single base-pair substitutions and deletions resulting in nucleotide mutations. Some other sequence editing examples include triplex forming oligonucliotides, small fragment homologous replacement, single-stranded DNA oligonucleotides, and adeno-associated virus (AAV) vectors.

The invention also is directed to genetic expression modification or mutagenesis, which may be carried out by delivery of a transgene that works in trans.

In one embodiment, RNA interference (RNAi) may be used to alter the expression of a gene. Single stranded mRNA can be regulated by the presence of sections of double stranded RNA (dsRNA) or small interfering RNA (siRNA). Both anti-sense and sense RNAs can be effective in inhibiting gene expression. siRNA mediates RNA interference and is created by cleavage of long dsDNA by the enzyme Dicer. RNAi can create genetic modification by triggering the degradation of mRNA's that are complementary to either strand of short dsRNA. When siRNA is associated with complementary single-stranded RNA it can signal for nuclease to degrade the mRNA. RNAi can also result in RNA silencing which occurs when the short dsRNA inhibits expression of a gene. Other forms of inhibitory RNA, such as small hairpin RNA (shRNA) are envisioned.

In another embodiment, the delivery of a transgene encoding a dominant negative protein may alter the expression of a target gene. Dominant negative proteins can inhibit the activity of an endogenous protein. One example is the expression a protein which contains the ligand binding site of an endogenous protein. The expressed dominant-negative protein “soaks up” all of the available ligand. The endogenous protein is therefore not activated, and the wild type function is knocked out or knocked down.

Other schemes based on these general concepts are within the scope and spirit of the invention, and are readily apparent to those skilled in the art.

The invention also provides methods for making homozygous mutations in rats by breeding a genetically modified rat which is heterozygous for a mutant allele with another genetically modified rat which is heterozygous for the same mutant allele. On average 25% of offspring of such matings are expected to produce animals that are homozygous for the mutant allele. Homozygous mutations are useful for discovering functions associated with the mutated gene.

The present invention is directed generally to reduction or inactivation of gene function or gene expression in cells in vitro and in multicellular organisms. The invention encompasses methods for mutating cells using one or more mutagens, particularly wherein at least one mutation is an insertion mutation, a deletion mutation, or a nucleic acid sequence mutation, to achieve a homozygous gene mutation or mutation of multiple genes required cumulatively to achieve a phenotype. The methods are used to create knock-outs, knock-downs, and other modifications in the same cell or organism.

The mutation can result in a change in the expression level of a gene or level of activity of a gene product. Activity encompasses all functions of a gene product, e.g. structural, enzymatic, catalytic, allosteric, and signaling. In one embodiment, mutation results in a decrease or elimination of gene expression levels (RNA and/or protein) or a decrease or elimination of gene product activity (RNA and/or protein). Most mutations will decrease the activity of mutated genes. However, both the insertional and physicochemical mutagens can also act to increase or to qualitatively change (e.g., altered substrate on binding specificity, or regulation of protein activity) the activity of the product of the mutated gene. Although mutations will often generate phenotypes that may be difficult to detect, most phenotypically detectable mutations change the level or activity of mutated genes in ways that are deleterious to the cell or organism.

As used herein, decrease means that a given gene has been mutated such that the level of gene expression or level of activity of a gene product in a cell or organism is reduced from that observed in the wild-type or non-mutated cell or organism. This is often accomplished by reducing the amount of mRNA produced from transcription of a gene, or by mutating the mRNA or protein produced from the gene such that the expression product is less abundant or less active.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a rat. Also disclosed are animals produced by the process of transfecting a cell within the animal any of the nucleic acid molecules disclosed herein, wherein the mammal is a rat.

Such methods are used to achieve mutation of a single gene to achieve a desired phenotype as well as mutation of multiple genes, required cumulatively to achieve a desired phenotype, in a rat cell or rat. The invention is also directed to methods of identifying one or more mutated genes, made by the methods of the invention, in rat cells and in rats, by means of a tagging property provided by the insertional mutagen(s). The insertional mutagen thus allows identification of one or more genes that are mutated by insertion of the insertional mutagen.

The invention is also directed to rat cells and rats created by the methods of the invention and uses of the rat cells and rats. The invention is also directed to libraries of rat cells created by the methods of the invention and uses of the libraries.

Drug Toxicology, Altered Drug and Chemical Metabolism-Associated Genes

The invention also features a novel genetically modified rat with a genetically engineered modification in a gene encoding a pain associated protein. In another aspect, the invention features a genetically modified rat, wherein a gene encoding pain protein is modified resulting in reduced pain protein activity. In preferred embodiments of this aspect, the genetically modified rat is homozygous for the modified gene. In other preferred embodiments, the gene encoding pain protein is modified by disruption, and the genetically modified rat has reduced pain protein activity. In yet another embodiment, the transgenic rat is heterozygous for the gene modification.

In another embodiment of this aspect of the invention, the invention features a nucleic acid vector comprising nucleic acid capable of undergoing homologous recombination with an endogenous pain gene in a cell, wherein the homologous recombination results in a modification of the pain gene resulting in decreased pain protein activity in the cell. In another aspect, the modification of the pain gene is a disruption in the coding sequence of the endogenous pain gene.

Another embodiment of this aspect of the invention features a rat cell, wherein the endogenous gene encoding pain protein is modified, resulting in reduced pain protein activity in the cell.

In certain embodiments, the reduced pain protein activity is manifested. In a related aspect, the invention features a rat cell containing an endogenous pain gene into which there is integrated a transposon comprising DNA encoding a gene trap and/or a selectable marker.

In another aspect, the invention features a rat cell containing an endogenous pain gene into which there is integrated a retrotransposon comprising DNA encoding a gene trap and/or a selectable marker. In another aspect, the invention features a rat cell containing an endogenous pain gene into which there is DNA comprising an insertion mutation in the pain gene. In another aspect, the invention features a rat cell containing an endogenous pain gene into which there is DNA comprising a deletion mutation in the pain gene. In another aspect, the invention features a rat cell containing an endogenous pain gene in which there has been nucleic acid sequence modification of the pain gene.

In another embodiment of the invention, the invention features a method for determining whether a compound is potentially useful for treating or alleviating the symptoms of a pain gene disorder, which includes (a) providing a cell that produces a pain protein, (b) contacting the cell with the compound, and (c) monitoring the activity of the pain protein, such that a change in activity in response to the compound indicates that the compound is potentially useful for treating or alleviating the symptoms of a pain gene disorder.

It is understood that simultaneous targeting of more than one gene may be utilized for the development of “knock-out rats” (i.e., rats lacking the expression of a targeted gene product), “knock-in rats” (i.e., rats expressing a fusion protein or a protein encoded by a gene exogenous to the targeted locus), “knock down rats” (i.e., rats with a reduced expression of a targeted gene product), or rats with a targeted gene such that a truncated gene product is expressed.

Rat models that have been genetically modified to alter pain gene expression may be used in in vivo assays to test for activity of a candidate pain modulating agent, or to further assess the role of pain gene in a pain pathway process such as T lymphocyte mediated apoptosis or native DNA autoantibody production. Preferably, the altered pain gene expression results in a detectable phenotype, such as decreased levels of P450 expression, bioavailability of a drug, increased susceptibility to toxicity, organ sequestration, compared to control animals having normal pain gene expression. The genetically modified rat may additionally have altered pain gene expression (e.g. pain gene knockout). In one embodiment, the genetically modified rats are genetically modified animals having a heterologous nucleic acid sequence present as an extrachromosomal element in a portion of its cells, i.e. mosaic animals (see, for example, techniques described by Jakobovits, 1994, Curr. Biol. 4:761-763) or stably integrated into its germ line DNA (i.e., in the genomic sequence of most or all of its cells). Heterologous nucleic acid is introduced into the germ line of such genetically modified animals by genetic manipulation of, for example, embryos or germ cells or germ cells precursors of the host animal.

Methods of making genetically modified rodents are well-known in the art (see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No. 4,945,050, by Sandford et al.; for genetically modified Drosophila see Rubin and Spradling, Science (1982) 218:348-53 and U.S. Pat. No. 4,670,388; for genetically modified insects see Berghammer A. J. et al., A Universal Marker for Genetically modified Insects (1999) Nature 402:370-371; for genetically modified Zebrafish see Lin S., Genetically modified Zebrafish, Methods Mol Biol. (2000); 136:375-3830); for microinjection procedures for fish, amphibian eggs and birds see Houdebine and Chourrout, Experientia (1991) 47:897-905; Hammer et al., Cell (1990) 63:1099-1112; and for culturing of embryonic stem (ES) cells and the subsequent production of genetically modified animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman genetically modified animals can be produced according to available methods (sec Wilmut, I. et al. (1997) Nature 385:810-813; and PCT International Publication Nos. WO 97/07668 and WO 97/07669).

In one embodiment, the genetically modified rat is a “knock-out” animal having a heterozygous or homozygous alteration in the sequence of an endogenous pain gene that results in a dysregulation of nervous system function, preferably such that pain gene expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the genetically modified host species. For example, a mouse drug trransporter gene is used to construct a homologous recombination vector suitable for altering an endogenous pain gene in the mouse genome. Detailed methodologies for homologous recombination in rodents are available (see Capecchi, Science (1989) 244:1288-1292; Joyner et al., Nature (1989) 338:153-156). Procedures for the production of non-rodent genetically modified mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as rats harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the genetically modified rat is a “knock-down” animal having an alteration in its genome that results in altered expression (e.g., decreased expression) of the pain gene, e.g., by introduction of mutations to the pain gene, or by operatively inserting a regulatory sequence that provides for altered expression of an endogenous copy of the pain gene.

Genetically modified rats can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a crc/loxP recombinase system is used to regulate expression of the transgene, animals containing transgenes encoding both the Cre recombinase and a selected protein are required. Such animals can be provided through the construction of “double” genetically modified animals, e.g., by mating two genetically modified animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified rats can be used in genetic studies to further elucidate the pain function pathways, as animal models of disease and disorders implicating dysregulated pain function, and for in vivo testing of candidate therapeutic agents, such as those identified in screens described below. The candidate therapeutic agents are administered to a genetically modified animal having altered pain function and phenotypic changes are compared with appropriate control animals such as genetically modified animals that receive placebo treatment, and/or animals with unaltered pain function that receive candidate therapeutic agent.

The invention also features novel genetically modified animals with a genetically engineered modification in the gene encoding pain proteins. In one aspect, the invention features a genetically modified non-human mammal, wherein a gene encoding a pain gene is provided as follows:

Intacellular Ca2+ regulation, temperature sensitization, axon guidance: TRP channels, Transient receptor potential channel (Trpc4).

The Trpc4 gene encodes a protein Transient receptor potential channel 4. Trpc4 is highly homologous to Trpc5. These nonselective cation channels are activated by G-protein coupled receptors (GPCRs) and tyrosine kinases and requires phospholipases c (PLC). TRP channels mediate a transmembrane flux of cations through electrochemical gradients, raising intracellular Ca2+ and Na+, depolarizing the cell and ultimately controlling neuronal potential and propogation. Cell changes in temperature are tightly associated with the opening of TRP channels. Interestingly, cell swelling may also activate TRP channel activation. These activation mechanisms add to the involvement in pain because of their association with hyperalgesia and inflammatory or traumatic induced pain exhibited in disease states such as diabetes. Cell bound guidance cues control axon guidance and facilitate axon interaction with targets. The tips of developing neurites modulate extension as a response to attractive or repellant stimulation. Axon stimulation can be effected by signals of pain and response to environmental sensitivity to temperature. Blocking TRP channels inhibits axon growth and activation promotes attractive steering; indicating that TRP channels regulate instructive Ca2+ signals in the nervous system. The role of TRP channels in axon guidance by Ca2+ flux which leads to instructive signaling validates Trpc4 as a TRP channel important in pain mechanisms such as sensory signaling in the peripheral nervous system. In order to produce effective animal models for pain Trpc4 knockout rats were produced by transposon mediated insertion.

Schwann cell development, axon-Schwann cell interaction, myelin mediated nerve conduction, pain: Neuregulin-1 (Nrg1)

Nrg1 encodes a protein NRG1, a key receptor in erbB signaling which plays a role in the interactions of peripheral axons and Schwann cells. Schwann cells produce myelin which regulates axon conduction and neuron-glia interaction. NRG1 induces neural crest cells, Schwann cell proliferation, survival of embryonic and immature Schwann cells, and cell migrations. Nrg1 expression is essential for Schwann cell development. NRG1-erbB signaling is critical for the development of myelin with normal thickness. Studies with dominant-negative (DN) erbB mice have elucidated that NRG1-erbB signaling in myelinating Schwann cells is critical for development of myelin sheaths. Nerve conduction velocity in NRG1-erbB signaling is severely reduced; however, the mice displayed enhanced sensitization to mechanical stimulation. In order to develop effective animal models for pain Nrg1 knockout rats were produced by transposon mediated insertion.

ErbB Signaling Defects, Non-Myelinating Schwann Cell Proliferation and Death Cycles, Sensory Defects: ErbB4

ErbB4 is a member of the type I receptor tyrosine kinase gene family which includes Egfr, and ErbB2 targets for anticancer drug Herceptin. ErbB4 is a transmembrane tyrosine receptor kinase which is instrumental in neuronal development. ErbB4 regulates non-myelinating Schwann cell proliferation and differentiation. In animal models which disrupted ErbB4 signaling non-myelination Schwann cells undergo “proliferate and die” cycles of which leads to dysregulation of sensory neurons and peripheral neuropathies. Memon et a.l. Brit. J. Cancer 91, 2004) discovered that NRG's and their receptors including ErbB4 are expressed in 91% of bladder cancers. However, invasive cancers display lower expression indicating that early loss of ErbB4 is a marker for bladder cancer development. By whole genome genetic mapping Silberberg et al. (Am. J. Med. Genet. 141B: 142-148, 2006) discovered three SNP's which resided in the third exon of ErbB4 and were tightly associated with Schizophrenia. In order to study the connection between ErbB4 mediated neuronal development and pain; rats with a transposon insertion within the gene rendering it expressionless were created.

Peroxisome proliferator-activated receptors (PPARs) consist of three isoforms (α, β/δ, γ). These ligand activated transcription factors are essential lipid metabolism regulators. However, PPAR administration in animal models has displayed an anti-inflammatory effect on neurodegeneration and autoimmune diseases. The success of neuroinflammatory treatment by PPAR's in animal models is provoked studies on their effect as treatments in human pain including inflammatory pain. The PPARα isoform has been shown to be upregulated in the spinal cords of rats with peripheral inflammation. PPARα was active in inducing hyperalgesia in rat models for neuroinflammation. Further, PPARα agonists reduced pain response behaviors in animal models for pain. PPARγ is also a neuroinflammatory pain related gene. Neuroprotection following cerebral ischemia is mediated by PPARγ inhibitors by blocking inflammation. In order to facilitate for effective study of PPAR involvement in pain PPARα, and PPARγ knockout rats were created.

The invention also features novel genetically modified cells and animals with a genetically engineered modification in a gene encoding for a pain protein. In one aspect, the invention features genetically modified rat cells or rats, wherein a gene modification occurs in a gene encoding a pain protein provided in Table 1:

TABLE 1

| Neu- | | Rat |
|---|---|---|
| ropathic | | Chromosomal |
| paingene | Function | Location |
| Nrg1 | Schwann cell development, proliferation, | 16q12.3 |
| | survival, migration. Generation of | |
| | function myelin. Nerve conduction | |
| | velocity and sensitivity, peripheral axon | |
| | and neuron-glia interactions. | |
| Trpc4 | Transmembrane cation flux, | 2q26 |
| | electrochemical gradient control, and | |
| | intracellular Ca2+ and Na+ concentrations. | |
| | Neuronal action potential enhancement, | |
| | axonal guidance and target sensory. | |
| Scn9a | Loss of function mutations result in | 8q24 |
| | channelopathy associated insensitivity to | |
| | pain; gain of function mutations result in | |
| | the pain disorder primary erythermalgia | |
| | due to enhanced sodium channels. | |
| | Mutations in this gene also cause extreme | |
| | pain disorder by inactivation of Na(v) 1.7 | |
| | channels. | |
| ErbB4 | Degradation of the sciatic nerve due to | 9q32 |
| | continuous cycles of non-myelination | |
| | Schwann cell proliferation and apoptosis. | |
| | Disruption of ErbB signaling results in a | |
| | progressively developed sensory defect | |
| | and neuropathic pain phenotype. | |
| Pnoc | Known as nociceptin, agonist for opioid | 1: |
| | receptor like-1. Induces allodynia and | (200918521- |
| | hyperalgesia. | 200928919) |
| | | bp |
| PPARα,, | PPAR's exhibit anti-neuroinflammatory | 7q34, 4q42 |
| PPARγ | and neuroportective properties in animal | |
| | models for neurodegeneration and | |
| | autoimmune diseases. Highly expressed in | |
| | the spinal cord of rats with peripheral | |
| | inflammation and elicits hyperalgesia | |

Methods

The methods used in the present invention are comprised of a combination of genetic introduction methods, genetic modification or mutagenesis mechanisms, and vector delivery methods. For all genetic modification or mutagenesis mechanisms one or more introduction and delivery method may be employed. The invention may include but is not limited to the methods described below.

Genetic Introduction Methods

In one introduction method, the pain gene is mutated directly in the germ cells of an adult animal. This method usually involves the creation of a transgenic founder animal by pronuclear injection. Rat oocytes are microinjected with DNA into the male pronucleus before nuclear fusion. The microinjected DNA creates a transgenic founder rat. In this method, a female rat is mated and the fertilized eggs are flushed from their oviducts. After entry of the sperm into the egg, the male and female pronuclei are separate entities until nuclear fusion occurs. The male pronucleus is larger are can be identified via dissecting microscope. The egg can be held in place by micromanipulation using a holding pipette. The male pronucleus is then microinjected with DNA that can be genetically modified. The microinjected eggs are then implanted into a surrogate pseudopregnant female which was mated with a vasectomized male for uterus preparation. The foster mother gives birth to transgenic founder animals. If the transgenic DNA encodes the appropriate components of a mutagenesis system, such as transposase and a DNA transposon, then mutagenesis will occur directly in the germ cells of founder animals and some offspring will contain new mutations. Chemical mutagenesis can also be used to cause direct germ line mutations.

In another introduction method, the pain gene is mutated in the early embryo of a developing animal. The mutant embryonic cells develop to constitute the germ cells of the organism, thereby creating a stable and heritable mutation. Several forms of mutageneis mechanisms can be introduced this way including, but not limited to, zinc finger nucleases and delivery of gene traps by a retrovirus.

In another introduction method, the pain gene is mutated in a pluripotent cell. These pluripotent cells can proliferate in cell culture and be genetically modified without affecting their ability to differentiate into other cell types including germ line cells. Genetically modified pluripotent cells from a donor can be microinjected into a recipient blastocyst, or in the case of spermatogonial stem cells can be injected into the rete testis of a recipient animal. Recipient genetically modified blastocysts are implanted into pseudopregnant surrogate females. The progeny which have a genetic modification to the germ line can then be established, and lines homozygous for the genetic modification can be produced by interbreeding.

In another introduction method, the pain gene is mutated in a somatic cell and then used to create a genetically modified animal by somatic cell nuclear transfer. Somatic cell nuclear transfer uses embryonic, fetal, or adult donor cells which are isolated, cultured, and/or modified to establish a cell line. Individual donor cells are fused to an enucleated oocyte. The fused cells are cultured to blastocyst stage, and then transplanted into the uterus of a pseudopregnant female. Alternatively the nucleus of the donor cell can be injected directly into the enucleated oocyte. See U.S. Appl. Publ. No. 20070209083.

Genetic Modification Methods

Mobile DNA Technology

DNA transposons are discrete mobile DNA segments that are common constituents of plasmid, virus, and bacterial chromosomes. These elements are detected by their ability to transpose self-encoded phenotypic traits from one replicon to another, or to transpose into a known gene and inactivate it. Transposons, or transposable elements, include a piece of nucleic acid bounded by repeat sequences. Active transposons encode enzymes (transposases) that facilitate the insertion of the nucleic acid into DNA sequences.

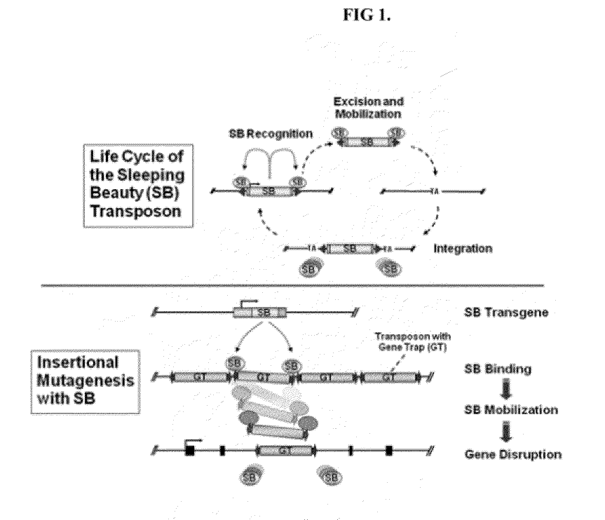

The lifecycle and insertional mutagenesis of DNA transposon Sleeping Beauty (SB) is depicted in FIG. 1. In its lifecycle, the SB encodes a transposase protein. That transposase recognizes the inverted terminal repeats (ITRs) that flank the SB transposon. The transposase then excises SB and reintegrates it into another region of the genome. Mutagenesis via Sleeping Beauty is depicted. The mechanism is similar to the life cycle, but transposase is not encoded by the transposon, but instead is encoded elsewhere in the genome

The Sleeping Beauty (SB) mutagenesis breeding and screening scheme is depicted in FIG. 2. One rat referred to as the “driver” rat contains the (SB) transposase within its genome. A second rat, the “donor” rat contains the transposon which has the transposase-recognizable inverted terminal repeats (ITRs). The two rats are bred to create the “seed” rat which has an active transposon containing transposase and ITRs. The transposon recognizes the ITRs, excises the transposon, and inserts it elsewhere in the rat's genome. This insertion event often disrupts coding, regulatory, and other functional regions in the genome to create knockout rat models. The “seed” rat is bred with wild type rats which beget heterozygous GI mutants. If the transposon has inserted into the genome, the event will be recorded via size comparison of DNA by Southern blot analysis. The exact location of the transposon insertion is determined by PCR-based amplification methods combined with sequencing of the DNA flanking the new insertion.

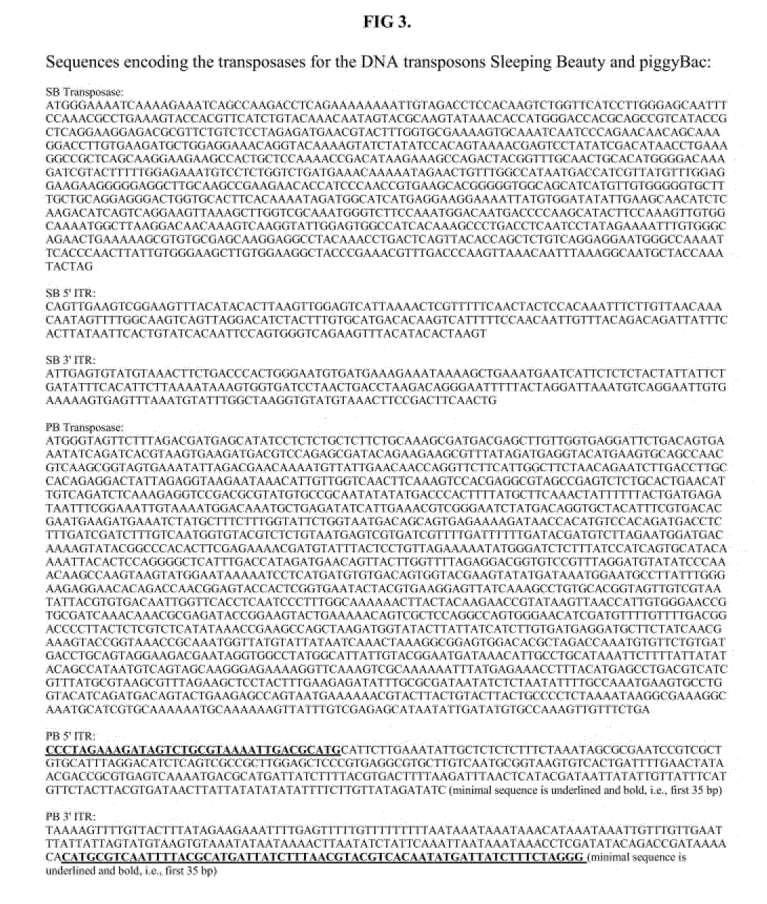

The sequences for the DNA transposons Sleeping Beauty (SB) piggyBac (PB) functional domains are shown in FIG. 3. The SB and PB transposase sequences encode the protein that recognizes the ITRs and carries out the excision and re-integration. The 3′ and 5′ ITRs are the flanking sequences which the respective transposases recognizes in order to carry out excision and reintegration elsewhere in the genome.

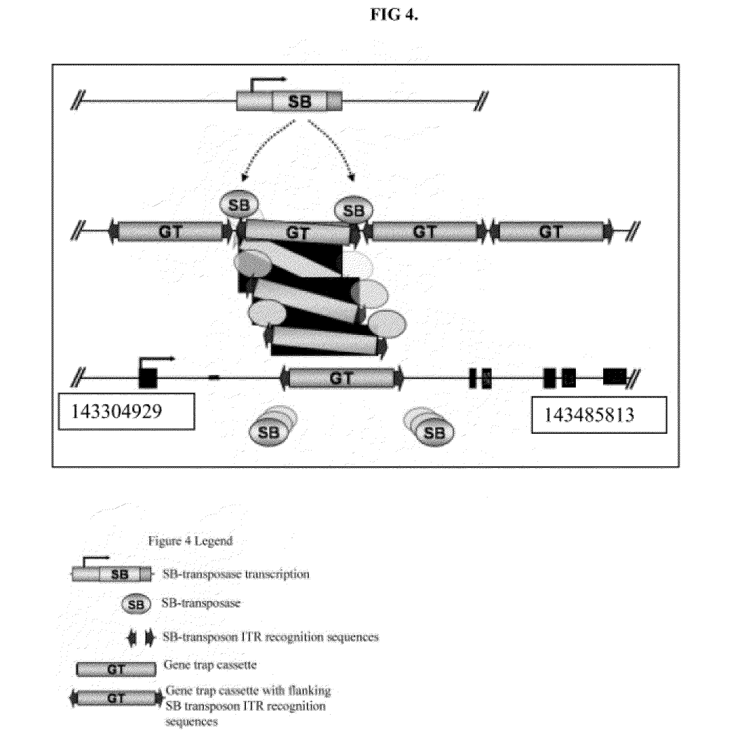

The DNA transposon Sleeping Beauty (SB) was used by the inventors to create a knockout rat in the Nrg1, Trpc4, ErbB4 genes. The mechanism is depicted in FIG. 4, and is the same as that described above. The transposase is encoded, and the protein recognizes the ITRs of the transposon. The transposon is then excised and reinserted into the first intron of the rat Nrg1, Trpc4, ErbB4 genes which resides on chromosome locations 16q12.3, 2q26, 9q32 respectively.

In another embodiment, the present invention utilizes the transposon piggyBac, and sequence configurations outside of piggyBac, for use as a mobile genetic element as described in U.S. Pat. No. 6,962,810. The Lepidopteran transposon piggyBac is capable of moving within the genomes of a wide variety of species, and is gaining prominence as a useful gene transduction vector. The transposon structure includes a complex repeat configuration consisting of an internal repeat (IR), a spacer, and a terminal repeat (TR) at both ends, and a single open reading frame encoding a transposase.

The Lepidopteran transposable element piggyBac transposes via a unique cut-and-paste mechanism, inserting exclusively at 5′ TTAA 3′ target sites that are duplicated upon insertion, and excising precisely, leaving no footprint (Elick et al., 1996b; Fraser et al., 1996; Wang and Fraser 1993).

In another embodiment, the present invention utilizes the Sleeping Beauty transposon system for genome manipulation as described, for example, in U.S. Pat. No. 7,148,203. In one embodiment, the system utilizes synthetic, salmonid-type Tel-like transposases with recognition sites that facilitate transposition. The transposase binds to two binding-sites within the inverted repeats of salmonid elements, and appears to be substrate-specific, which could prevent cross-mobilization between closely related subfamilies of fish elements.

In another aspect of this invention, the invention relates to a transposon gene transfer system to introduce DNA into the DNA of a cell comprising: a nucleic acid fragment comprising a nucleic acid sequence positioned between at least two inverted repeats wherein the inverted repeats can bind to a SB protein and wherein the nucleic acid fragment is capable of integrating into DNA of a cell; and a transposase or nucleic acid encoding a transposase. In one embodiment, the transposase is provided to the cell as a protein and in another the transposase is provided to the cell as nucleic acid. In one embodiment the nucleic acid is RNA and in another the nucleic acid is DNA. In yet another embodiment, the nucleic acid encoding the transposase is integrated into the genome of the cell. The nucleic acid fragment can be part of a plasmid or a recombinant viral vector. Preferably, the nucleic acid sequence comprises at least a portion of an open reading frame and also preferably, the nucleic acid sequence comprises at least a regulatory region of a gene. In one embodiment the regulatory region is a transcriptional regulatory region and the regulatory region is selected from the group consisting of a promoter, an enhancer, a silencer, a locus-control region, and a border element. In another embodiment, the nucleic acid sequence comprises a promoter operably linked to at least a portion of an open reading frame.

In the transgene flanked by the terminal repeats, the terminal repeats can be derived from one or more known transposons. Examples of transposons include, but are not limited to the following: Sleeping Beauty (Izsvak Z, Ivies Z. and Plasterk R H. (2000) Sleeping Beauty, a wide host-range transposon vector for genetic transformation in vertebrates. J. Mol. Biol. 302:93-102), mosl (Bessereau J L, et al. (2001) Mobilization of a Drosophila transposon in the Caenorhabditis elegans germ line. Nature. 413(6851):70-4; Zhang L, et al. (2001) DNA-binding activity and subunit interaction of the mariner transposase. Nucleic Acids Res.29(17):3566-75, piggyBac (Tamura T. et al. Germ line transformation of the silkworm Bombyx mori L. using a piggyBac transposon-derived vector. Nat Biotechnol. 2000 January; 18(1):81-4), Himar1 (Lampe D J, et al. (1998) Factors affecting transposition of the Himar1 mariner transposon in vitro. Genetics. 149(11):179-87), Hermes, Tol2 element, Pokey, Tn5 (Bhasin A, et al. (2000) Characterization of a Tn5 pre-cleavage synaptic complex. J Mol Biol 302:49-63), Tn7 (Kuduvalli P N, Rao J E, Craig N L. (2001) Target DNA structure plays a critical role in Tn7 transposition. EMBO J 20:924-932), Tn916 (Marra D, Scott J R. (1999) Regulation of excision of the conjugative transposon Tn916. Mol Microbiol 2:609-621), Tel/mariner (Izsvak Z, Ivies Z4 Hackett P B. (1995) Characterization of a Tel-like transposable element in zebrafish (Danio rerio). Mol. Gen. Genet. 247:312-322), Minos and S elements (Franz G and Savakis C. (1991) Minos, a new transposable element from Drosophila hydei, is a member of the Tc1-like family of transposons. Nucl. Acids Res. 19:6646; Merriman P J, Grimes C D, Ambroziak J, Hackett D A, Skinner P, and Simmons M J. (1995) S elements: a family of Tc1-like transposons in the genome of Drosophila melanogaster. Genetics 141:1425-1438), Quetzal elements (Ke Z, Grossman G L, Cornel A J, Collins F H. (1996) Quetzal: a transposon of the Tel family in the mosquito Anopheles albimanus. Genetica 98:141-147); Txr elements (Lam W L, Seo P, Robison K, Virk S, and Gilbert W. (1996) Discovery of amphibian Tc1-like transposon families. J Mol Biol 257:359-366), Tel-like transposon subfamilies (Ivies Z, Izsvak Z, Minter A, Hackett P B. (1996) Identification of functional domains and evolution of Tel-like transposable elements. Proc. Natl. Acad Sci USA 93: 5008-5013), Tc3 (Tu Z. Shao H. (2002) Intra- and inter-specific diversity of Tc-3 like transposons in nematodes and insects and implications for their evolution and transposition. Gene 282:133-142), ICESt1 (Burrus V et al. (2002) The ICESt1 element of Streptococcus thermophilus belongs to a large family of integrative and conjugative elements that exchange modules and change their specificity of integration. Plasmid. 48(2): 77-97), maT, and P-element (Rubin G M and Spradling A C. (1983) Vectors for P element-mediated gene transfer in Drosophila. Nucleic Acids Res. 11:6341-6351). These references are incorporated herein by reference in their entirety for their teaching of the sequences and uses of transposons and transposon ITRs.

Translocation of Sleeping Beauty (SB) transposon requires specific binding of SB transposase to inverted terminal repeats (ITRs) of about 230 bp at each end of the transposon, which is followed by a cut-and-paste transfer of the transposon into a target DNA sequence. The ITRs contain two imperfect direct repeats (DRs) of about 32 bp. The outer DRs are at the extreme ends of the transposon whereas the inner DRs are located inside the transposon, 165-166 bp from the outer DRs. Cui et al. (J. Mol Biol 318:1221-1235) investigated the roles of the DR elements in transposition. Within the 1286-bp element, the essential regions are contained in the intervals bounded by coordinates 229-586, 735-765, and 939-1066, numbering in base pairs from the extreme 5′ end of the element. These regions may contain sequences that are necessary for transposase binding or that are needed to maintain proper spacing between binding sites.

Transposons are bracketed by terminal inverted repeats that contain binding sites for the transposase. Elements of the IR/R subgroup of the Tc1/mariner superfamily have a pair of transposase-binding sites at the ends of the 200-250 bp long inverted repeats (IRs) (Izsvak, et al. 1995). The binding sites contain short, 15-20 bp direct repeats (DRs). This characteristic structure can be found in several elements from evolutionarily distant species, such as Minos and S elements in flies (Franz and Savakis, 1991; Merriman et al, 1995), Quetzal elements in mosquitoes (Ke et al, 1996), Txr elements in frogs (Lam et al, 1996) and at least three Tc1-like transposon subfamilies in fish (Ivies et al., 1996), including SB [Sleeping Beauty] and are herein incorporated by reference.

Whereas Tc1 transposons require one binding site for their transposase in each IR, Sleeping Beauty requires two direct repeat (DR) binding sites within each IR, and is therefore classified with Tc3 in an IR/DR subgroup of the Tc1/mariner superfamily (96,97). Sleeping Beauty transposes into TA dinucleotide sites and leaves the Tc1/mariner characteristic footprint, i.e., duplication of the TA, upon excision. The non-viral plasmid vector contains the transgene that is flanked by IR/DR sequences, which act as the binding sites for the transposase. The catalytically active transposase may be expressed from a separate (trans) or same (cis) plasmid system. The transposase binds to the IR/DRs, catalyzes the excision of the flanked transgene, and mediates its integration into the target host genome.

Naturally occurring mobile genetic elements, known as retrotransposons, are also candidates for gene transfer vehicles. This mutagenesis method generally involves the delivery of a gene trap.

Retrotransposons are naturally occurring DNA elements which are found in cells from almost all species of animals, plants and bacteria which have been examined to date. They are capable of being expressed in cells, can be reverse transcribed into an extrachromosomal element and reintegrate into another site in the same genome from which they originated.

Retrotransposons may be grouped into two classes, the retrovirus-like LTR retrotransposons, and the non-LTR elements such as human L1 elements, Neurospora TAD elements (Kinsey, 1990, Genetics 126:317-326), I factors from Drosophila (Bucheton et al., 1984, Cell 38:153-163), and R2Bm from Bombyx mori (Luan et al., 1993, Cell 72: 595-605). These two types of retrotransposon are structurally different and also retrotranspose using radically different mechanisms.

Unlike the LTR retrotransposons, non-LTR elements (also called polyA elements) lack LTRs and instead end with polyA or A-rich sequences. The LTR retrotransposition mechanism is relatively well-understood; in contrast, the mechanism of retrotransposition by non-LTR retrotransposons has just begun to be elucidated (Luan and Eickbush, 1995, Mol. Cell. Biol. 15:3882-3891; Luan et al., 1993, Cell 72:595-605). Non-LTR retrotransposons can be subdivided into sequence-specific and non-sequence-specific types. L1 is of the latter type being found to be inserted in a scattered manner in all human, mouse and other mammalian chromosomes.

Some human L1 elements (also known as a LINEs) can retrotranspose (express, cleave their target site, and reverse transcribe their own RNA using the cleaved target site as a primer) into new sites in the human genome, leading to genetic disorders.

Further included in the invention are DNAs which are useful for the generation of mutations in a cell. The mutations created are useful for assessing the frequency with which selected cells undergo insertional mutagenesis for the generation of genetically modified animals and the like. Engineered L1 elements can also be used as retrotransposon mutagens. Sequences can be introduced into the L1 that increases its mutagenic potential or facilitates the cloning of the interrupted gene. DNA sequences useful for this application of the invention include marker DNAs, such as GFP, that are specifically engineered to integrate into genomic DNA at sites which are near to the endogenous genes of the host organism. Other potentially useful DNAs for delivery are regulatory DNA elements, such as promoter sequences, enhancer sequences, retroviral LTR elements and repressors and silencers. In addition, genes which are developmentally regulated are useful in the invention.

Viral Mutagenesis Methods

Viral vectors are often created using a replication defective virus vector with a genome that is partially replaced by the genetic material of interest (e.g., gene trap, selectable marker, and/or a therapeutic gene). The viral vector is produced by using a helper virus to provide some of the viral components that were deleted in the replication defective virus, which results in an infectious recombinant virus whose genome encodes the genetic material of interest. Viral vectors can be used to introduce an insertion mutation into the rat's genome. Integration of the viral genetic material is often carried out by the viral enzyme integrase. Integrase brings the ends of viral DNA together and converts the blunt ends into recessed ends. Integrase creates staggered ends on chromosomal DNA. The recessed ends of the viral DNA are then joined with the overhangs of genomic DNA, and the singlestranded regions are repaired by cellular mechanisms. Some recombinant virus vectors are equipped with cell uptake, endosomal escape, nuclear import, and expression mechanisms allowing the genetic material of interest to be inserted and expressed in the rat's genome. The genetic material introduced via viral vectors can genetically modify the rat's genome but is not limited to disrupting a gene, inserting a gene to be expressed, and by delivery of interfering RNA. Viral vectors can be used in multiple methods of delivery. The most common mode of delivery is the microinjection of a replication deficient viral vector (e.g. retroviral, adenoviral) into an early embryo (1-4 day) or a onecell pronuclear egg. After viral vector delivery, the embryo is cultured in vitro and transferred to recipient rats to create genetically modified progeny.

In one embodiment, insertion mutations can be created by delivery of a gene trap vector into the rat genome. The gene trap vector consists of a cassette that contains selectable reporter tags. Upstream from this cassette is a 3′ splice acceptor sequence. Downstream from the cassette lays a termination sequence poly adenine repeat tail (polyA). The splice acceptor sequence allows the gene trap vector to be spliced into chromosomal mRNA. The polyA tail signals the premature interruption of the transcription. The result is a truncated mRNA molecule that has decreased function or is completely non-functional. The gene trap method can also be utilized to introduce exogenous DNA into the genome.

In another embodiment an enhancer trap is used for insertional mutagenesis. An enhancer trap is a transposable element vector that carries a weak minimal promoter which controls a reporter gene. When the transposable element is inserted the promoter drives expression of the reporter gene. The expression of the reporter gene also displays the expression patterns of endogenous genes. Enhancer trapping results in genetic modification and can be used for gain-of-function genetics. The Gal4-mediated expression system is an example of an enhancer trap.

Further included are one or more selectable marker genes. Examples of suitable prokaryotic marker genes include, but are not limited to, the ampicillin resistance gene, the kanamycin resistance gene, the gene encoding resistance to chloramphenicol, the lacZ gene and the like. Examples of suitable eukaryotic marker genes include, but are not limited to, the hygromycin resistance gene, the green fluorescent protein (GFP) gene, the neomycin resistance gene, the zeomycin gene, modified cell surface receptors, the extracellular portion of the IgG receptor, composite markers such as beta-geo (a lac/neo fusion) and the like.

In one embodiment, the gene trap will need to be integrated into the host genome and an integrating enzyme is needed. Integrating enzymes can be any enzyme with integrating capabilities. Such enzymes are well known in the art and can include but are not limited to transposases, integrases, recombinases, including but not limited to tyrosine site-specific recombinases and other site-specific recombinases (e.g., cre), bacteriophage integrases, retrotransposases, and retroviral integrases.

The integrating enzymes of the present invention can be any enzyme with integrating capabilities. Such enzymes are well known in the art and can include but are not limited to transposases (especially DDE transposases), integrases, tyrosine site-specific recombinases and other site-specific recombinases (e.g., cre), bacteriophage integrases, integrons, retrotransposases, retroviral integrases and terminases.

Disclosed are compositions, wherein the integrating enzyme is a transposase. It is understood and herein contemplated that the transposase of the composition is not limited and to any one transposase and can be selected from at least the group consisting of Sleeping Beauty (SB), Tn7, Tn5, mosl, piggyBac, Himar1, Hermes, Tol2, Pokey, Minos, S elements, P-elements, ICESt1, Quetzal elements, Tn916, maT, Tel/mariner and Tc3.

Where the integrating enzyme is a transposase, it is understood that the transposase of the composition is not limited and to any one transposase and can be selected from at least the group consisting of Sleeping Beauty (SB), Tn7, Tn5, Tn916, Tel/mariner, Minos and S elements, Quetzal elements, Txr elements, maT, most, piggyBac, Himar1, Hermes, Tol2, Pokey, P-elements, and Tc3. Additional transposases may be found throughout the art, for example, U.S. Pat. No. 6,225,121, U.S. Pat. No. 6,218,185 U.S. Pat. No. 5,792,924 U.S. Pat. No. 5,719,055, U.S. Patent Application No. 20020028513, and U.S. Patent Application No. 20020016975 and are herein incorporated by reference in their entirety. Since the applicable principal of the invention remains the same, the compositions of the invention can include transposases not yet identified.

Also disclosed are integrating enzymes of the disclosed compositions wherein the enzyme is an integrase. For example, the integrating enzyme can be a bacteriophage integrase. Such integrase can include any bacteriophage integrase and can include but is not limited to lamda bacteriophage and mu bacteriophage, as well as Hong Kong 022 (Cheng Q., et al. Specificity determinants for bacteriophage Hong Kong 022 integrase: analysis of mutants with relaxed core-binding specificities. (2000) Mol Microbiol. 36(2):424-36.), HP1 (Hickman, A. B., et al. (1997). Molecular organization in site-specific recombination: The catalytic domain of bacteriophage HP1 integrase at 2.7 A resolution. Cell 89: 227-237), P4 (Shoemaker, N B, et al. (1996). The Bacteroides mobilizable insertion element, NBU1, integrates into the 3′ end of a Leu-tRNA gene and has an integrase that is a member of the lambda integrase family. J Bacteriol. 178(12):3594-600.), P1 (Li Y, and Austin S. (2002) The P1 plasmid in action: time-lapse photomicroscopy reveals some unexpected aspects of plasmid partition. Plasmid. 48(3):174-8.), and T7 (Rezende, L. F., et al. (2002) Essential Amino Acid Residues in the Single-stranded DNA-binding Protein of Bacteriophage T7. Identification of the Dimer Interface. J. Biol. Chem. 277, 50643-50653.). Integrase maintains its activity when fused to other proteins.

Also disclosed are integrating enzymes of the disclosed compositions wherein the enzyme is a recombinase. For example, the recombinase can be a Cre recombinase, Flp recombinase, HIN recombinase, or any other recombinase. Recombinases are well-known in the art. An extensive list of recombinases can be found in Nunes-Duby S E, et al. (1998) Nuc. Acids Res. 26(2): 391-406, which is incorporated herein in its entirety for its teachings on recombinases and their sequences.

Also disclosed are integrating enzymes of the disclosed compositions wherein the enzyme is a retrotransposase. For example, the retrotransposase can be a GATE retrotransposase (Kogan G L, et al. (2003) The GATE retrotransposon in Drosophila melanogaster: mobility in heterochromatin and aspects of its expression in germ line tissues. Mol Genet Genomics. 269(2):234-42).

Other general techniques for integration into the host genome include, for example, systems designed to promote homologous recombination. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

Zinc Finger Nucleases

In another method, a zinc finger nuclease creates site-specific deletions via double-stranded DNA breaks that are repaired by non-homologous end joining (NHEJ). Zinc finger nucleases may also be used to create an insertion mutation by combining the ZFN with a homologously integrating cassette to create an insertion in the genomic DNA. Therefore, this genetic modification method can be used for both targeted (site-specific) DNA insertions and targeted DNA deletions. In one embodiment, the method involves transformation of a cell with a nucleic acid or mRNA construct minimally comprising DNA encoding a chimeric zinc finger nuclease (ZFN), which can be used to create a DNA deletion. In another embodiment, a second DNA construct can be provided that will serve as a template for repair of the cleavage site by homologous recombination. In this embodiment, a DNA insertion may be created. The DNA insertion may contain a gene trap cassette. In one embodiment, this method can be combined with spermatogonia) stem cell technology or embryonic stem cell technology, as mentioned above. In another embodiment, this method can be combined with mobile DNA technology. This technique can also be done directly in the rat embryo.

Nucleic Acid Modification Methods

In one embodiment, a random mutation is created with a chemical mutagen and then a screen is performed for insertions in a particular pain gene. Chemical mutagens such as methane-sulfonic acid ethylester (EMS), N-ethyl-N-nitrosourea (ENU), diepoxyoctane and UV/trimethylpsorlalen may be employed to create nucleic acid sequence mutations.

Sequence editing methods can also be used that involve the delivery of small DNA fragments, hybrid DNA/RNA molecules, and modified DNA polymers to create sequence mismatches and nucleic acid mutations. RNA/DNA hybrids are molecules composed of a central stretch of DNA flanked by short RNA sequences that form hairpin structures. The RNA/DNA hybrids can produce single base-pair substitutions and deletions resulting in nucleotide mutations. Some other sequence editing examples include triplex forming oligonucleotides, small fragment homologous replacement, single stranded DNA oligonucleotides, and adeno-associated virus (AAV) vectors.

The invention also is directed to genetic expression modification or mutagenesis by delivery of a transgene that works in trans.

In one genetic modification method, RNA interference may be used to alter the expression of a gene. In another genetic modification method, the delivery of a transgene encoding a dominant negative protein may alter the expression of a target gene.

Vector Delivery Methods

The mutagenesis methods of this invention may be introduced into one or more cells using any of a variety of techniques known in the art such as, but not limited to, microinjection, combining the nucleic acid fragment with lipid vesicles, such as cationic lipid vesicles, particle bombardment, electroporation, DNA condensing reagents (e.g., calcium phosphate, polylysine or polyethyleneimine) or incorporating the nucleic acid fragment into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector.

DNA or other genetic material may be delivered through viral and non-viral vectors. These vectors can carry exogenous DNA that is used to genetically modify the genome of the rat. For example Adenovirus (AdV), Adeno-associated virus (AAV), and Retrovirus (RV) which contain LTR regions flanking a gene trap, transgene, cassette or interfering RNA are used to integrate and deliver the genetic material. Another delivery method involves non-viral vectors such as plasmids used for electroporation and cationic lipids used for lipofection. The non-viral vectors usually are engineered to have mechanisms for cell uptake, endosome escape, nuclear import, and expression. An example would be a non-viral vector containing a specific nuclear localization sequence and sequence homology for recombination in a targeted region of the genome.

There are a number of compositions and methods which can be used to deliver nucleic acids to cells, either in vitro or in vivo. For example, the nucleic acids can be delivered through a number of direct delivery systems such as, electroporation, lipofection, calcium phosphate precipitation, plasmids, cosmids, or via transfer of genetic material in cells or carriers such as cationic liposomes. Appropriate means for transfection, including chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed non-viral vectors for example, lipids such as liposomes, such as cationic liposomes (e.g., DOTMA, DOPE, DC-cholesterol) or anionic liposome, or polymersomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the vector can be administered as a component of a microcapsule that can be targeted to specific cell types, such as macrophages, or where the diffusion of the compound or delivery of the compound from the microcapsule is designed for a specific rate or dosage.

In the methods described above, which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

These vectors may be targeted to a particular cell type via antibodies, receptors, or receptor ligands. The following references are examples of the use of this technology to target specific proteins to tumor tissue and are incorporated by reference herein (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid-mediated drug targeting to colonic carcinoma), receptor-mediated targeting of DNA through cell specific ligands, lymphocyte-directed tumor targeting, and highly specific therapeutic retroviral targeting of murine glioma cells in vivo. The following references are examples of the use of this technology to target specific proteins to tumor tissue and are incorporated by reference herein (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Nucleic acids that are delivered to cells which are to be integrated into the host cell genome typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Nrg1 Domains and Loss of Function Mutations

Rattus norvegicus Neuregulin-1 (NRG1) is a 662 amino acid (AA) protein. The protein consists of multiple conserved domains and processing sites. Molecular processing sequences include AA: propeptide, 1-13; pro-neuregulin-1 membrane bound isoform, 14-662; neuregulin-1, 14-264. Conserved domains occur between AA: extracellular, 14-265; internal signal sequence 266-288; cytoplasmic, 289-662; Ig-like C2 type, 37-128; EGF like, 178-222; Ser/The rich, 165-177. Amino acid modification sites occur at AA: N-linked glycosylation, 120, 126, 164; disulfide bond, 57-112, 182-196, 190-210, 212-221. The Nrg1 gene mRNA consists of 3272 base pairs with a coding sequence between base pairs 345-2255. A highly conserved region which is essential for proper erbB signaling is 215 bp in length between by 2601-2815.

Lee et al. (Nature. 378:394-398, 1995) found that NRG1 is essential for Schwann cell development. Stefansson et al. (Am. J. Hum. Genet. 71: 877-892, 2002) discovered by genome wide association between Nrg1 and Schizophrenia.

TABLE
Amino Acid changes resulting in pain pathway modification.
This table displays some amino acid changes that are predicted
to disrupt NRG1 activity.

| Amino Acid | NRG1 functional domain effected |
|---|---|
| 1-13 | propeptide formation |
| 14-662 | Proneurgulin formation |
| 14-264 | Neuregulin function, Schwann cell development |
| 14-265 | Extracellular domain function |
| 266-288 | Signaling, cellular localization |
| 289-662 | Cytoplasmic, regulation of interactions and |
| | trafficking, glia interaction |
| 37-128 | Immuno globulin like domain |
| 178-222 | EGF-like, erbB signaling and binding domain |
| 120, 126, 164 | Post transcriptional modification disruption, |
| | decrease in function |
| 57-112, 182- | Disulfide bond disruption, decrease in function |
| 196, 190-210, | |
| 212-221 | |

Trpc4 Domains and Loss of Function Mutations

Rattus norvegicus Transient receptor potential channel 4 (TRPC4) is a 977 amino acid (AA) protein. The multipass membrane protein consists of multiple conserved domains. Cytoplasmic domains AA: 1-329, 384-436, 491-511, 621-974; transmembrane domains AA: 330-350, 363-383, 437-457, 470-490, 512-532, 660-620; extracellular domains AA: 351-362, 458-469, 533-599; ANK repeats AA: 69-98, 141-170; binding domain for ITPR1, 2, 3 receptors AA: 615-977; binding domain for NHERF PDV domain AA: 975-977; modified residues, phosphoserine AA: 193, 195.

TABLE
Amino Acid changes resulting in pain pathway modification.
This table displays some amino acid changes that are predicted
to disrupt TRPC4 activity.

| Amino Acid | TRPC4 functional domain effected |
|---|---|
| 1-329, 384- | Cypoplasmic domain potentiation disrupted, |
| 436, 491-511, | axon guidance, ion channel opening, Ca2+ and |
| 621-974 | Na+ flux disruption |
| 330-350, | Transmembrane anchoring function, disruption |
| 363-383, | of structure function relationships, ion channel |
| 437-457, | opening, Ca2+ and Na+ flux disruption, axon |
| 470-490, | guidance |
| 512-532, | |
| 600-620 | |
| 351-362, | Extracellular signaling, erbB signaling, axon |
| 458-469, | guidance, ion channel opening, Ca2+ and Na+ |
| 533-599 | flux disruption, axon guidance |
| 69-98, | ANK repeat interactions |
| 141-170 | |
| 615-977 | ITPR1, 2, 3 binding and interaction disruption |
| 975-977 | NHERF PDZ domain binding and interaction |
| | disruption |
| 193, 195 | Post-transcriptional modification interaction |
| | disruption |

ErbB4 Domains and Loss of Function

Rattus norvegicus v-erb-a erythroblastic leukemia viral oncogene homolog 4 (ErbB4) is a 1308 amino acid (AA) protein. The proteins signal peptide is AA 1-25. The ErBB4 protein consists of multiple conserved domains. Cytoplasmic domains AA: 676-1308; transmembrane domains AA: 652-675; extracellular domains AA: 26-651; protein kinase AA: 718-985; ATP nucleotide binding AA: 724-732; WW1 binding AA: 1032-1035; WW2 binding AA: 1298-1301; PDZ binding AA: 1306-1308; cysteine rich 186-334, 496-633; Active sites proton acceptor AA: 843, ATP binding AA: 751; modified residues, phosphotyrosine AA: 733, 1162, 1188, 1258, 1284; glycosylation AA: 138, 174, 253, 358, 410, 473, 495, 548, 576, 620; disulfide bonds occur continuously between AA: 29-633. Silberberg et al. discovered three SNP's in the ErbB4 third exon that were closely associated with the development of Schizophrenia.

Amino Acid changes resulting in pain pathway modification.
This table displays some amino acid changes that are predicted
to disrupt ErbB4 activity.

| Amino Acid | ErbB4 functional domain effected |
|---|---|
| 676-1308 | Cytoplasmic domain interactions |
| 652-675 | Structure function dysregulation |
| 26-651 | Extracellular interactions, cellular signaling |
| 718-985 | Protein kinase function and transactivation |
| | signaling |
| 724-732 | Nucleotide binding defects |
| 1032-1035 | Binding and interaction with WWOX |
| 1298-1301 | Binding and interaction with WWOX |
| 1306-1308 | PDZ binding |
| 186-334 | Cysteine rich |
| 496-633 | Cysteine rich |
| 843 | Proton acceptor |
| 751 | ATP binding |
| 733, 1162, | Protein folding |
| 1188, 1258, | |
| 1284 | |
| 138, 174, | Protein folding |
| 253, 358, | |
| 410, 473, | |
| 495, 548, | |
| 576, 620 | |
| 29-633 | Disulfide bond formation |
| 26-651 | Schizophrenia related |

Nrg1, Trpc4, ErbB4 Phenotypes

The Nrg1, Trpc4, ErbB4 activity resulting from a loss of function in one or several Nrg1, Trpc4, ErbB4 effectors has completely different and variable phenotypes; some resulting in less sensitivity to pain response. Complete loss of function or “knockout” of Nrg1, Trpc4, ErbB4 resulting in loss of function in all of its effectors always results in hyposensitivity to pain response. These defects resulting from non-functional Nrg1, Trpc4, ErbB4 are known to affect the pain signaling pathway, axonal signaling, Ca2+ or Na+ flux signaling, Schwann cell development, signaling, survival, myelin development in known animal models. This pain signaling pathway alteration affects the sensitivity to induced pain responses, spontaneous pain, hyperalgesia, temperature and light induced pain, sudden pain attacks, disease state such as diabetic and inflammatory induced pain and drug induced pain. Animal models exhibiting defects in the Nrg1, Trpc4, ErbB4 gene are models of pain.

TABLE
Pain Gene Phenotypes

| Gene | Pain induction | KO pain response phenotype |
|---|---|---|
| Nrg1 | Mechanical von Frey filament, | Almost a 100% reduction in |
| | heat induced, light induced | sensitivity response to |
| | | induced pain when compared |
| | | to WT control rats |
| Trpc4 | Mechanical von Frey filament, | A large reduction in |
| | heat induced, light induced, | sensitivity to induced |
| | drug induced, acetone, | pain occurred in |
| | disease state (diabetes) | mechanical, light, heat, |
| | induced, and drug induced. | acetone induced. |
| ErbB4 | Heat and cold plate exposure | Sciatic nerve degradation, |
| | followed by paw withdrawal | delayed response to heat |
| | latency measurement. | and cold sensation. |
| Pparα/γ | Induced hyperalgesia | PPAR Knockouts have |
| | and nociception. SNI | reduced autonomic |
| | constriction | nociceptive behaviors and |
| | | reduced hyperalgesic |
| | | responses to SNI |
| | | constriction |

CLUSTAL 2.0.10 multiple sequence alignment of rat and mouse Neuregulin 1, Transient receptor potential cation channel, subfamily C, member 4, and v-erb-a erythroblastic leukemia viral oncogene homolog amino acid sequence. The sequence alignment shows close homology between the mouse and rat Nrg1, Trpc4, ErbB4 sequence. The homology of conserved domains and knowledge of insertion mutagenesis allows evidence that mutagenesis has created a total knockout rat in Nrg1, Trpc4, ErbB4.

| | | |
|---|---|---|
| NRG1 | | |
| rattus | GCGGCCGCAGCTGCCGGGAGATGCGAGCGCAGACCGGATTGTGATCACCTTTCCCTCTTC | 60 |
| mus | ------------------------------------------------------------ | |
| rattus | GGGCTGTAAGAGAGCGAGACAAGCCACCGAAGCGAGGCCACTCCAGAGCCGGCAGCGGAG | 120 |
| mus | ------------------------------------------------------------ | |
| rattus | GGACCCGGGACACTAGAGCAGCTCCGAGCCACTCCAGACTGAGCGGACGCTCCAGGTGAT | 180 |
| mus | ---------ATGGAGATTTATCCCCCAGACATGTCTGAGGGAGCTGGCGGG--AGGTCCT | 49 |
| | * * * ** ** ** * ** **** * ** **** * | |
| rattus | CGAGTCCACGCTGCTTCCTGCAGGCGACAGGCGACGCCTCCCGAGCAGC--CCGGCCACT | 238 |
| mus | CCAGCCC---CTCCACTCAGCTGAGTGCAGACC-CATCTCTCGATGGGCTTCCGGCAGCG | 105 |
| | * ** ** ** * * ** * *** * * *** *** ** ***** * | |
| rattus | GGCTCTTCCCC---TCCTGGGACAAACTTTTCTGCAAGCCCTTGGACCAAACTTGTCGCG | 295 |
| mus | GAACATATGCCAGACACCCACACAGAAGATGGGAGAAGCCCT-GGAC------TCCTGGG | 158 |
| | * * ** * *** * * ******* **** * * * | |
| rattus | CGTCACCGTCACCCAACCGGGTCCGCGTAGAGCGCTCATCTTCGGCGAGATGTCTGAGCG | 355 |
| mus | CCTGGCCGT-GCCCTGCTGTGTCTGCCTGGAA------------GCGGAGCGTCTCAGAG | 205 |
| | * * **** *** * * *** ** * ** *** **** ** * | |
| rattus | CAAAGAAGGCAGAGGCAAGGGGAAGGGCAAGAAGAAGGACCGGGGATCCCGCGGGAAGCC | 415 |
| mus | --GGTGCCTCAACTCCGAGAAGATCTGCATTGTTCCCATTCTGGCTTGTCTAGTAAGCCT | 253 |
| | ** * ** ** *** * ** * * * * * | |
| rattus | CGGGCCCGCCGAGGGCGACCCGAGCCCAGCACTGCCTCCCAGATTGAAAGAAATGAAGAG | 475 |
| mus | CTGCCTCTGCATTGCTGGCCTAAAGTGGGTATTTG---------TGGACAAGATATTCGA | 314 |
| | * * * * * * * ** * * * * ** * * ** | |
| rattus | CCAGGAGTCAGCTGCAGGCTCCAAGCTAGTGCTCCGGTGCGAAACCAGCTCCGAGTACTC | 535 |
| mus | ATACGACTCTCCTAC-----CCACCTTGACCCTGGGGGGTTAGGCCAGGACC--CTGTTA | 367 |
| | * ** ** ** * *** * ** ** * * **** ** * * | |
| rattus | CTCACTCAGATTCAAATGGTTCAAGAATGGGAACGAGCTGAACCGCAAAA-ATAAACCAG | 594 |
| mus | TTTCTCTGGATCCAACGGCTGCCTCCGCTGTTCTGGTCTCATCCGAGGCATACACTTCAC | 427 |
| | * *** *** * * * * * ** * *** * * * ** | |
| rattus | AAAACATCAAGATACAGAA-GAAGCCAGGGAAGTCAGAGCTTCGAATTAACAAAGCATCC | 653 |
| mus | CTGTCTCTAAGGCTCAGTCTGAAGCCGAGG--CTCATGTTACAGGGCAAGGTGACCATGT | 485 |
| | * *** *** ****** ** *** * * * *** | |
| rattus | CTGGCTGACTCTGGAGAGTATATGTGCAAAGTGATCAGCAAGTTAGGAAATGACAGTGC- | 712 |
| mus | CGCTGTGGCCTCTGAACCT-----TCCGCAGTACCCACCCGG--AAGAACCGGCTGTCTG | 538 |
| | * ** * ** * * * *** ** * * * *** * * ** | |
| rattus | CTCTGCCAACATCACCATTGTTGAGTCAAACGAGTTCATCACTGGCATGCCAGCCTCGAC | 772 |
| mus | CTTTTCCTCCCTTACACTCCACTCCACCGCCCTTCCCTTCTCCAGCTCGGACCCCTGAGG | 598 |
| | ** * ** * * ** * * * * ** * ** * *** | |
| rattus | TGAGACAGCCTATGT---GTCCTCAGAGTCTCCCATTAGAATCTCAGTTTCAACAGAAGG | 829 |
| mus | TGAGAACACCCAAGTCAGGAACTCAGCCACAAACAACAGAAACTAA---TCTGCAAACTG | 655 |
| | ***** ** * ** * ***** * ** **** ** * ** ** * * | |
| rattus | CGCAAACACTTCTTCATCCACATCAACATCCACGACTGGGACCAGCCATCTCATAAAGTG | 889 |
| mus | CTCCTAAACTT-----TCCACATCTACATCCACGACTGGGACCAGCCATCTCATAAAGTG | 710 |
| | * * * **** ******** *********************************** | |
| rattus | TGCGGAGAAGGAGAAAACTTTCTGTGTGAATGGGGGCGAGTGCTTCACGGTGAAGGACCT | 949 |
| mus | TGCGGAGAAGGAGAAAACTTTCTGTGTGAATGGAGGCGAGTGCTTCATGGTGAAGGACCT | 770 |
| | ********************************* ************* ************ | |
| rattus | GTCAAACCCGTCAAGATACTTGTGCAAGTGCCCAAATGAGTTTACTGGTGATCGTTGCCA | 1009 |
| mus | GTCAAACCCCTCAAGATACTTGTGCAAGTGCCCAAATGAGTTTACTGGTGATCGTTGCCA | 830 |
| | ********* ************************************************** | |
| rattus | AAACTACGTAATGGCCAGCTTCTACAA------------------------AGCGGAGGA | 1045 |
| mus | AAACTACGTAATGGCCAGCTTCTACAAGCATCTTGGGATTGAATTTATGGAAGCGGAGGA | 890 |
| | *************************** ********* | |
| rattus | ACTCTACCAGAAGAGGGTGCTGACAATTACTGGCATCTGTATCGCCCTGCTGGTGGTCGG | 1105 |
| mus | GCTCTACCAGAAGAGGGTACTGACAATTACTGGCATCTGTATCGCCCTGTTGGTGGTCGG | 950 |
| | ***************** ****************************** ********** | |
| rattus | CATCATGTGTGTGGTGGCCTACTGCAAAACCAAGAAGCAGCGGCAGAAGCTTCATGATCG | 1165 |
| mus | CATCATGTGTGTGGTGGCCTACTGCAAAACCAAGAAACAGCGGCAGAAGCTTCATGATCG | 1010 |
| | ************************************ *********************** | |
| rattus | GCTTCGGCAGAGTCTTCGGTCAGAACGGAGCAACCTGGTGAACATAGCGAATGGGCCTCA | 1225 |
| Titus | GCTCCGGCAGAGCCTTCGGTCAGAACGAAACAACATGGTGAACATAGCGAATGGCCCTCA | 1070 |
| | *** ******** ************** * **** ******************* ***** | |
| rattus | CCACCCAAACCCACCGCCAGAGAACGTGCAGCTGGTGAATCAATACGTATCTAAAAACGT | 1285 |
| mus | CCATCCAAACCCACCACCAGAGAATGTGCAACTGGTGAATCAATATGTATCTAAAAACGT | 1130 |
| | *** *********** ******** ***** ************** ************** | |
| rattus | CATCTCCAGTGAGCATATTGTTGAGAGAGAAGTGGAGACTTCCTTTTCCACCAGTCATTA | 1345 |
| mus | CATCTCCAGTGAGCATATTGTGGAGAGAGAAGTGGAGACCTCCTTTTCCACCAGTCACTA | 1190 |
| | ********************* ***************** ***************** ** | |
| rattus | CACTTCCACAGCCCATCACTCCACGACTGTCACCCAGACTCCTAGTCACAGCTGGAGTAA | 1405 |
| mus | CACTTCCACAGCTCATCACTCCACGACTGTCACCCAGACTCCTAGTCACAGCTGGAGTAA | 1250 |
| | ************ *********************************************** | |
| rattus | TGGGCACACGGAGAGCGTCATTTCAGAAAGCAACTCCGTAATCATGATGTCTTCGGTAGA | 1465 |
| mus | TGGGCACACAGAAAGCATCATTTCAGAAAGCCACTCTGTAATCATGATGTCATCGGTAGA | 1310 |
| | ********* ** *** ************** **** ************** ******** | |
| rattus | GAACAGCAGGCACAGCAGTCCCGCCGGGGGCCCACGAGGACGTCTTCATGGCCTGGGAGG | 1525 |
| mus | GAACAGCAGGCACAGCAGCCCAGCTGGGGGCCCACGAGGACGTCTTCATGGCCTGGGAGG | 1370 |
| | ****************** ** ** *********************************** | |
| rattus | CCCTCGTGA---TAACAGCTTCCTCAGGCATGCCAGAGAAACCCCTGACTCCTACAGAGA | 1582 |
| mus | CCCTCGCGAATGTAACAGCTTCCTCAGGCATGCCAGAGAAACCCCTGACTCCTACAGAGA | 1430 |
| | ****** ** ************************************************ | |
| rattus | CTCTCCTCATAGCGAAAGGTATGTATCAGCCATGACCACCCCGGCTCGTATGTCACCTGT | 1642 |
| mus | CTCTCCTCATAGTGAAAGGTATGTATCAGCCATGACCACCCCGGCTCGTATGTCACCTGT | 1490 |
| | ************ *********************************************** | |
| rattus | AGATTTCCACACGCCAAGCTCCCCTAAATCGCCCCCTTCGGAAATGTCTCCACCCGTGTC | 1702 |
| mus | AGATTTCCACACGCCAAGCTCCCCTAAATCGCCCCCTTCGGAAATGTCTCCACCCGTGTC | 1550 |
| | ************************************************************ | |
| rattus | CAGCATGACGGTGTCCATGCCCTCTGTGGCAGTCAGCCCCTTTGTGGAAGAAGAGAGGCC | 1762 |
| mus | CAGCATGACGGTGTCCATGCCCTCTGTGGCAGTCAGCCCCTTTGTGGAAGAAGAGAGGCC | 1610 |
| | ************************************************************ | |
| rattus | TCTGCTGCTTGTGACGCCACCAAGGCTACGGGAGAAGAAATATGATCATCACCCCCAGCA | 1822 |
| mus | TCTGCTTCTTGTGACGCCACCGAGGCTACGGGAGAAGAAGTATGATCATCACCCCCAGCA | 1670 |
| | ****** ************** ***************** ******************** | |
| rattus | ACTCAACTCCTTTCATCACAACCCTGCACATCAGAGTACCAGCCTCCCCCCTAGCCCACT | 1882 |
| mus | ACTCAACTCCTTTCATCACAACCCTGCACATCAGAGTACCAGCCTCCCCCCTAGCCCATT | 1730 |
| | ********************************************************** * | |
| rattus | GAGGATAGTGGAGGATGAGGAGTACGAGACGACCCAGGAGTATGAGTCAGTTCAAGAGCC | 1942 |
| mus | GAGGATAGTGGAGGATGAGGAATACGAAACGACCCAGGAGTATGAGCCAATTCAAGAGCC | 1790 |
| | ********************* ***** ****************** ** ********** | |
| rattus | CGTTAAGAAAGTCACCAATAGCCGGCGGGCCAAAAGAACCAAGCCCAATGGCCACATTGC | 2002 |
| mus | TATTAAGAAAGTCACCAATAGCCGGCGGGCCAAAAGAACCAAGCCCAATGGCCACATTGC | 1850 |
| | ********************************************************** | |
| rattus | CAATAGGTTGGAAATGGACAGCAACACAAGTTCTGTGAGCAGTAACTCAGAAAGTGAGAC | 2062 |
| mus | CAATAGGTTGGAAATGGACAGCAACCCAAGTTCTGTGAGCAGTAACTCAGAAAGTGAGAC | 1910 |
| | ************************* ********************************** | |
| rattus | AGAAGACGAAAGAGTAGGTGAAGACACACCATTCCTGGGCATACAGAACCCCCTGGCAGC | 2122 |
| mus | AGAAGATGAAAGAGTAGGTGAAGATACACCATTCCTGGGCATACAGAACCCCCTGGCAGC | 1970 |
| | ****** ***************** *********************************** | |
| rattus | CAGCCTTGAGGTGGCCCCTGCCTTCCGTCTGGCTGAGAGCAGGACTAACCCAGCAGGCCG | 2182 |
| mus | CAGCCTTGAGGTGGCCCCTGCCTTCCGTCTGGCTGAGAGCAGGACTAACCCAGCAGGCCG | 2030 |
| | ************************************************************ | |
| rattus | CTTCTCCACACAGGAGGAATTACAGGCCAGGCTGTCTAGTGTAATCGCTAACCAAGACCC | 2242 |
| mus | CTTCTCCACACAGGAAGAATTACAGGCCAGGCTGTCTAGTGTAATCGCTAACCAAGACCC | 2090 |
| | *************** ******************************************** | |
| rattus | TATTGCTGTATAAAACCTAAATAAACACATAGATTCACCTGTAAAACTTTATTTTATATA | 2302 |
| mus | TATTGCTGTATAA----------------------------------------------- | 2103 |
| | ************* | |
| rattus | ATAAAGTATTTCACCTTAAATTAAACAATTTATTTTATTTTAGCAGTTCTGCAAATAGAA | 2362 |
| mus | ------------------------------------------------------------ | |
| rattus | AACAGGAAGAAAAAAAAACTTTTATAAATTAAATATATGTATGTAAAAATGTGTTATGTG | 2422 |
| mus | ------------------------------------------------------------ | |
| rattus | CCATATGTAGCAATTTTTTTACAGTATTTCAAAAACGAGAAAGATATCAATGGTGCCTTT | 2482 |
| mus | ------------------------------------------------------------ | |
| rattus | ATGTTCTGTTATGTCGAGAGCAAGTTTTATAAAGTTATGGTGATTTCTTTTTCACAGTAT | 2542 |
| mus | ------------------------------------------------------------ | |
| rattus | TTCAGCAAAACCTGCCATATATTCAGTTTCTGCTGGCTTTTTGTGCATTGCATTATGATG | 2602 |
| mus | ------------------------------------------------------------ | |
| rattus | TTGACTGGATGTATGGTTTGCAAGGCTAGCAGCTCGCTCGTGTTCTCTCTCTCTCTCTCT | 2662 |
| mus | ------------------------------------------------------------ | |
| rattus | CTCTCTCTCTCTCTGTCTCTCTCTCTGTCTCTCTCTCTCTCTCTCTCTCTCTCTCTCTCT | 2722 |
| mus | ------------------------------------------------------------ | |
| rattus | CTCTCTCTCTCTCTCTCTCTCTGTCTCTCTCTCTGCTTCCCGTAGCTCCCAACCAGTACT | 2782 |
| mus | ------------------------------------------------------------ | |
| rattus | GTCTTGGACTGGCACATCCATCCAAATACCTTTCTACTTTGTATGAAGTTTTCTTTGCTT | 2842 |
| mus | ------------------------------------------------------------ | |
| rattus | TCCCAATATGAAATGAGTTCTCTCTACTCTGTCAGCCAAAGGTTTGCTTCACTGGACTCT | 2902 |
| mus | ------------------------------------------------------------ | |
| rattus | GAGATAATAGTAGACCCAGCAGCATGCTACTATTACGTATAGCAGGAAACTGCACCAAGT | 2962 |
| mus | ------------------------------------------------------------ | |
| rattus | AATGTCCAATAATAGGAAGAAAGTAATACTGTGATTTAAAAAAAAAAACAAACTATATTA | 3022 |
| mus | ------------------------------------------------------------ | |
| rattus | TTAATCAGAAGACAGCTTGCTCTTGGTAAAAGGAGCTACCATTGACTCTAATTTTGACTT | 3082 |
| mus | ------------------------------------------------------------ | |
| rattus | TTTAGTTATTGTTCTTGACAAAGAGTAACAGCTTCAAGTACAGCCTAGAAAAAAAAATGG | 3142 |
| mus | ------------------------------------------------------------ | |
| rattus | GTTCTGGCCTGCTATCAGGATAAATCTATCGACGTAGATAGATTCAACTCAGTTTCACTT | 3202 |
| mus | ------------------------------------------------------------ | |
| rattus | TCTGTCTTGGGGGAAATGATCCAGCCACTCATATGACGACCAACCAACCACAGGTGCCTC | 3262 |
| mus | ------------------------------------------------------------ | |
| rattus | TGCTCCCTGT | 3272 |
| mus | ---------- | |
| Trpc4 | | |
| rattus | ------------------------------------------------------------ | |
| mus | GTTTTTTTCCCCCTTGGAATGCTCCAAAAAACTCGGTAGCGACTACGGAAACCCCATCGG | 60 |
| rattus | -------CAGCTGCGCTAGCACCAGGCACAGCACTGGTGCCACGCGCCCGCCGAGCCCAC | 53 |
| mus | AACTGACCAGCTGCGCTAGCACCAGGCACAGCACTGGTGCTGCGCGCTCGCCGAGCCCAC | 120 |
| | ********************************* ***** ************ | |
| rattus | CGCGGTCACTTCAGCCACCAGATTGCAACTTTGCGGAGATGATG---GACTAGCATGGCC | 110 |
| mus | CTCGGTCACTTCAACCACCAGATTGCAACTTTGCGGAGATGATGATGGACTAGCATGGCC | 180 |
| | * *********** ****************************** ************* | |
| rattus | TGAAGCATGGCTCAGTTCTATTACAAACGAAATGTCAACGCCCCCTACCGAGACCGCATC | 170 |
| mus | TGAAGCATGGCTCAGTTCTATTACAAAAGAAATGTCAACGCCCCCTACAGAGACCGCATC | 240 |
| | *************************** ******************** *********** | |
| rattus | CCACTGAGGATCGTCAGGGCAGAATCTGAACTCTCACCATCAGAGAAAGCCTACTTGAAT | 230 |
| mus | CCACTGAGGATTGTCAGAGCAGAATCTGAGCTCTCACCATCAGAGAAAGCCTACTTGAAT | 300 |
| | *********** ***** *********** ****************************** | |
| rattus | GCCGTGGAAAAGGGGGACTATGCAAGCGTCAAGAAATCTCTGGAGGAAGCCGAGATTTAT | 290 |
| mus | GCTGTGGAGAAGGGGGACTATGCAAGCGTCAAGAAGTCTCTGGAGGAAGCTGAGATTTAT | 360 |
| | ** ***** ************************** ************** ********* | |
| rattus | TTTAAAATCAACATTAACTGCATTGACCCCCTTGGGAGGACTGCTCTTCTCATTGCCATT | 350 |
| mus | TTTAAAATCAACATTAACTGCATCGACCCCCTGGGAAGGACCGCCCTCCTCATTGCCATT | 420 |
| | *********************** ******** ** ***** ** ** ************ | |
| rattus | GAAAATGAGAACCTGGAGCTGATTGAACTGTTGTTGAGTTTCAATCTCTATGTTGGCGAT | 410 |
| mus | GAAAATGAGAATCTGGAGCTTATTGAACTATTGTTGAGTTTCAATGTCTATGTAGGCGAT | 480 |
| | *********** ******** ******** *********************** ****** | |
| rattus | GCGCTACTTCACGCCATCAGGAAAGAGGTGGTTGGAGCCGTGGAGCTACTGCTGAACCAC | 470 |
| mus | GCGCTGCTTCACGCCATCAGAAAAGAGGTGGTTGGAGCCGTCGAGCTACTGCTGAACCAC | 540 |
| | ***** ************** *************************************** | |
| rattus | AAAAAAGCCCAGCGGAGAGAAGCAGGTGCCTCCCATCCTCCTTGACAAACAGTTCTCTGA | 530 |
| mus | AAAAA-GCCAAGTGGAGAGAAGCAGGTGCCTCCCATTCTCCTTGATAAACAGTTCTCTGA | 599 |
| | ***** *** ** *********************** ******** ************** | |
| rattus | ATTCACCCCAGACATCACGCCTATCATCTTGGCTGCACATACAAATAATTATGAGATAAT | 590 |
| mus | ATTCACTCCGGACATCACACCCATCATCTTGGCTGCACATACAAATAATTACGAGATAAT | 659 |
| | ****** ** ******** ** ***************************** ******** | |
| rattus | CAAACTCTTGGTCCAGAAGGGTGTCTCGGTGCCCAGACCCCACGAGGTCCGCTGTAACTG | 650 |
| mus | CAAACTTTTGGTTCAGAAAGGTGTCTCAGTGCCCAGACCCCACGAGGTCCGCTGTAACTG | 719 |
| | ****** ***** ***** ******** ******************************** | |
| rattus | TGTTGAGTGTGTCTCCAGCTCAGACGTGGACAGCCTCAGGCACTCACGGTCCAGGCTCAA | 710 |
| mus | TGTTGAGTGTGTCTCCAGCTCGGATGTGGACAGCCTCAGGCATTCACGGTCCAGGCTCAA | 779 |
| | ********************* ** ***************** ***************** | |
| rattus | CATCTACAAGGCTTTGGCCAGCCCCTCGCTCATTGCGCTGTCAAGTGAAGACCCTTTCCT | 770 |
| mus | CATCTACAAGGCCTTGGCCAGCCCCTCGCTCATTGCCCTGTCAAGCGAAGACCCTTTCCT | 839 |
| | ************ *********************** ******** ************** | |
| rattus | CACCGCCTTTCAGTTAAGCTGGGAGCTGCAAGAACTGAGTAAGGTGGAGAATGAATTCAA | 830 |
| mus | TACTGCCTTTCAGTTAAGTTGGGAGCTGCAAGAACTCAGCAAGGTGGAGAACGAATTCAA | 899 |
| | ** ************** ***************** ** *********** ******** | |
| rattus | GTCGGAGTATGAGGAGCTGTCTAGACAGTGCAAACAGTTTGCTAAGGACCTCCTAGATCA | 890 |
| mus | GTCGGAGTATGAGGAGCTGTCTAGACAGTGCAAACAATTTGCCAAGGACCTCCTAGATCA | 959 |
| | ************************************ ***** ***************** | |
| rattus | GACACGGAGTTCCAGAGAGCTGGAAATCATTCTTAATTACCGTGATGACAATAGCCTGAT | 950 |
| mus | GACACGGAGTTCCAGAGAGCTGGAAATCATTCTTAATTACCGTGATGACAATAGTCTGAT | 1019 |
| | ****************************************************** ***** | |
| rattus | CGAAGAACAGAGTGGAAATGATCTTGCGAGGCTAAAATTAGCCATTAAGTACCGTCAAAA | 1010 |
| mus | CGAAGAACAGAGTGGAAATGATCTTGCAAGGCTAAAATTAGCCATTAAGTACCGTCAAAA | 1079 |
| | *************************** ******************************** | |
| rattus | AGAGTTTGTTGCTCAGCCCAACTGCCAGCAGCTGCTTGCTTCCCGCTGGTACGATGAGTT | 1070 |
| mus | AGAGTTTGTTGCTCAGCCCAACTGCCAGCAGCTGCTCGCTTCCCGCTGGTACGATGAGTT | 1139 |
| | ************************************ *********************** | |
| rattus | CCCAGGCTGGAGGAGAAGACACTGGGCGGTGAAGATGGTGACATGTTTCATAATAGGACT | 1130 |
| mus | CCCAGGCTGGAGGAGAAGACACTGGGCGGTGAAGATGGTGACGTGTTTCATAATAGGACT | 1199 |
| | ****************************************** ***************** | |
| rattus | ACTCTTCCCCGTCTTCTCCGTGTGCTACCTGATAGCTCCCAAAAGCCCACTTGGACTGTT | 1190 |
| mus | ACTCTTCCCCGTCTTCTCCGTGTGCTACCTGATAGCTCCCAAAAGCCCACTTGGACTGTT | 1259 |
| | ************************************************************ | |
| rattus | CATCAGAAAGCCATTTATCAAGTTTATCTGCCACACAGCCTCCTATCTGACCTTTTTGTT | 1250 |
| mus | CATCAGAAAGCCATTTATCAAGTTTATCTGCCACACAGCCTCCTATCTGACCTTTTTGTT | 1319 |
| | ************************************************************ | |
| rattus | TCTGCTGCTGCTAGCCTCTCAGCACATCGACAGGT------------------------- | 1285 |
| mus | TCTGCTGCTGCTAGCCTCTCAGCACATCGACAGGTCAGACTTGAACAGGCAAGGTCCACC | 1379 |
| | *********************************** | |
| rattus | -------------------------------------------TTTATATGGGGAGAGAT | 1302 |
| mus | ACCAACCATCGTGGAGTGGATGATATTACCGTGGGTCCTGGGTTTTATATGGGGAGAGAT | 1439 |
| | ***************** | |
| rattus | TAAACAGATGTGGGATGGCGGACTTCAGGATTACATCCACGACTGGTGGAATCTAATGGA | 1362 |
| mus | TAAACAGATGTGGGATGGCGGACTCCAGGATTACATCCATGACTGGTGGAATCTAATGGA | 1499 |
| | ************************ ************** ******************** | |
| rattus | CTTTGTGATGAACTCCTTGTATCTGGCGACAATCTCCTTGAAGATTGTCGCATTTGTAAA | 1422 |
| mus | CTTTGTGATGAACTCCTTGTATCTGGCAACAATCTCCTTGAAGATTGTCGCGTTTGTAAA | 1559 |
| | *************************** *********************** ******** | |
| rattus | GTACAGTGCTCTGAACCCACGGGAATCATGGGACATGTGGCACCCCACCCTGGTGGCAGA | 1482 |
| mus | GTACAGTGCTCTGAACCCACGGGAATCATGGGACATGTGGCACCCCACCCTGGTGGCAGA | 1619 |
| | ************************************************************ | |
| rattus | GGCTTTATTCGCAATTGCAAACATCTTCAGTTCCCTCCGCCTGATCTCTCTGTTCACTGC | 1542 |
| mus | GGCATTATTTGCTATTGCAAACATCTTCAGTTCCCTCCGCCTGATCTCTCTGTTCACTGC | 1679 |
| | *** ***** ** *********************************************** | |
| rattus | CAATTCTCACCTGGGGCCTCTGCAGATATCTCTGGGAAGAATGCTCCTGGACATCCTAAA | 1602 |
| mus | CAATTCTCACCTGGGGCCTCTGCAGATATCTCTGGGAAGGATGCTTCTGGACATCCTGAA | 1739 |
| | *************************************** ***** *********** ** | |
| rattus | GTTCTTATTCATATACTCCCTCGTGCTGCTAGCTTTTGCAAATGGCCTAAATCAACTGTA | 1662 |
| mus | GTTCTTGTTCATCTACTGCCTCGTGCTGCTAGCTTTTGCAAATGGCCTAAATCAGCTGTA | 1799 |
| | ****** ***** ***************************************** ***** | |
| rattus | CTTCTACTATGAAGAAACGAAGGGGTTAAGCTGCAAAGGCATACGGTGCGAAAAACAGAA | 1722 |
| mus | CTTTTACTATGAAGAAACAAAGGGGCTAAGCTGCAAAGGCATCCGGTGCGAGAAACAGAA | 1859 |
| | *** ************** ****** **************** ******** ******** | |
| rattus | CAACGCGTTCTCCACGTTATTTGAGACTCTACAGTCCCTGTTTTGGTCAATATTTGGACT | 1782 |
| mus | CAACGCGTTTTCCACGTTATTCGAGACACTACAGTCCCTGTTTTGGTCAATATTTGGACT | 1919 |
| | ********* *********** ***** ******************************** | |
| rattus | CATCAATCTCTATGTTACCAATGTCAAAGCCCAGCATGAGTTCACTGAGTTTGTTGGGGC | 1842 |
| mus | CATCAATCTCTATGTTACCAATGTCAAAGCCCAGCACGAGTTCACTGAGTTTGTTGGGGC | 1979 |
| | ************************************ *********************** | |
| rattus | CACCATGTTTGGCACATATAACGTCATCTCTCTGGTTGTCCTCCTGAACATGCTGATCGC | 1902 |
| mus | CACCATGTTTGGCACATATAATGTCATCTCTCTGGTTGTCCTGCTGAACATGTTAATTGC | 2039 |
| | ********************* ******************** ********* * ** ** | |
| rattus | TATGATGAATAATTCTTACCAACTAATTGCCGACCACGCAGATATAGAGTGGAAATTTGC | 1962 |
| mus | TATGATGAATAATTCTTACCAACTAATTGCCGACCATGCAGATATAGAATGGAAATTTGC | 2099 |
| | ************************************ *********** *********** | |
| rattus | TCGAACAAAGCTTTGGATGAGCTACTTTGAAGAAGGGGGTACCCTGCCTACACCTTTCAA | 2022 |
| mus | TCGAACAAACCTTTGGATCACCTACTTTGAAGAAGGAGGTACCCTGCCTACACCTTTCAA | 2159 |
| | ************************************ *********************** | |
| rattus | TGTCATCCCAAGCCCCAAGTCCCTGTGGTACCTGGTCAAGTGGATATGGACGCACTTATG | 2082 |
| mus | TGTCATCCCAAGCCCCAAGTCCCTGTGGTACCTGGTCAAGTGGATATGGACACACTTATG | 2219 |
| | *************************************************** ******** | |
| rattus | TAAGAAAAAGATGAGAAGAAAGCCAGAAAGCTTTGGGACAATTGGGCGGCGTGCTGCTGA | 2142 |
| mus | TAAGAAAAAAATGAGAAGGAAGCCAGAAAGCTTCGGGACAATTGGGCGGCGTGCTGCTGA | 2279 |
| | ********* ******** ************** ************************** | |
| rattus | TAACTTGAGAAGGCATCACCAATACCAAGAGGTGATGAGGAATCTGGTGAAGCGGTACGT | 2202 |
| mus | TAACTTGAGAAGACATCACCAATACCAAGAGGTGATGAGGAACCTGGTGAAGCGGTACGT | 2339 |
| | ************ ***************************** ***************** | |
| rattus | GGCAGCCATGATCAGAGAGGCAAAAACTGAAGAAGGCTTGACAGAGGAGAATGTTAAGGA | 2262 |
| mus | GGCAGCCATGATCAGAGAGGCAAAAACCGAAGAAGGCTTGACGGAGGAGAATGTTAAGGA | 2399 |
| | *************************** ************** ***************** | |
| rattus | ACTAAAGCAAGACATTTCTAGCTTCCGCTTCGAAGTTCTGGGATTGCTCCGGGGAAGCAA | 2322 |
| mus | ACTAAAGCAAGACATTTCTAGCTTCCGCTTCGAAGTTCTGGGATTGCTCAGAGGAAGCAA | 2459 |
| | ************************************************* * ******** | |
| rattus | GCTCTCAACAATACAGTCAGCCAACGCAGCGAGTTCAGCCAGCTCCGCGGACTCCGATGA | 2382 |
| mus | GCTCTCTACAATACAGTCAGCCAACGCGGCGAGTTCAGC---------GGACTCCGACGA | 2510 |
| | ****** ******************** *********** ********* ** | |
| rattus | GAAGAGCCACAGCGAAGGTAATGGCAAGGACAAGAGAAAGAATCTCAGCCTCTTTGATTT | 2442 |
| mus | GAAGAGCCAGAGCGAAGGTAATGGCAAGGACAAGAGAAAGAATCTCAGCCTCTTTGATTT | 2570 |
| | ********* ************************************************** | |
| rattus | AACCACTCTGATCCACCCGCGGTCGGCAGTCATTGCCTCCGAGAGACATAACCTAAGCAA | 2502 |
| mus | AACCACTCTGATCCACCCGCGGTCGGCAGCCATTGCCTCCGAGAGACATAACCTAAGCAA | 2630 |
| | ***************************** ****************************** | |
| rattus | TGGTTCTGCCCTGGTGGTGCAGGAGCCGCCCAGGGAGAAGCAGAGGAAAGTGAATTTTGT | 2562 |
| mus | TGGTTCCGCCCTGGTGGTGCAGGAGCCGCCCAGGGAGAAGCAGAGGAAAGTGAATTTTGT | 2690 |
| | ****** ***************************************************** | |
| rattus | GGCTGATATCAAAAACTTCGGGTTATTTCATAGACGGTCAAAGCAAAATGCTGCTGAGCA | 2622 |
| mus | GGCTGATATCAAAAACTTCGGGTTATTTCATAGACGGTCAAAACAAAATGCTGCTGAGCA | 2750 |
| | ****************************************** ***************** | |
| rattus | AAACGCAAACCAAATCTTCTCTGTTTCAGAAGAAATTACTCGTCAACAGGCGGCAGGAGC | 2682 |
| mus | AAACGCAAACCAAATCTTCTCTGTTTCAGAAGAAATTACTCGTCAACAGGCGGCAGGAGC | 2810 |
| | ************************************************************ | |
| rattus | ACTTGAGAGAAATATCCAACTGGAATCCCAAGGATTAGCTTCACGGGGTGACCGCAGCAT | 2742 |
| mus | ACTTGAGCGAAATATCGAACTGGAATCCAAAGGATTAGCTTCACGGGGTGACCGCAGCAT | 2870 |
| | ******* ******** *********** ******************************* | |
| rattus | TCCTGGTCTCAATGAACAGTGTGTGCTAGTAGACCATAGAGAAAGGAATACGGACACTTT | 2802 |
| mus | TCCTGGTCTCAATGAACAGTGTGTGCTAGTAGACCATAGAGAAAGGAATACGGACACTTT | 2930 |
| | ************************************************************ | |
| rattus | GGGTTTACAGGTAGGCAAGAGAGTGTGCTCCTCCTTCAAGTCGGAGAAGGTGGTGGTGGA | 2862 |
| mus | GGGTTTACAGGTAGGCAAGAGAGTGTGCTCCACCTTCAAGTCGGAGAAGGTGGTGGTGGA | 2990 |
| | ******************************* **************************** | |
| rattus | AGACACCGTCCCTATTATACCAAAGGAGAAACACGCCCAGGAGGAGGACTCAAGCATAGA | 2922 |
| mus | AGACACCGTCCCTATTATACCAAAGGAGAAACACGCCCACGAGGAGGACTCGAGCATAGA | 3050 |
| | *************************************** *********** ******** | |
| rattus | TTATGATTTAAGCCCCACGGACACAGTTGCCCATGAAGATTATGTGACCACGAGATTGTG | 2982 |
| mus | CTATGACTTAAGCCCCACGGACACAGCTGCCCACGAAGATTATGTGACCACAAGATTGTG | 3110 |
| | ***** ******************* ****** ***************** ******** | |
| rattus | ACAACTTGGAGAAGGAGTGTTTACCATACCTATACATATTTTCCATAGTGCTCTGGGCAG | 3042 |
| mus | ACC-CTTGG---AGGAGTGTTTACCATACCTATACATATTTTCCATAGTGCTCTGGGCAG | 3166 |
| | ** ***** ************************************************ | |
| rattus | GCAAAATGTATGAAATTACATTATCAAATGCTAATTTACACTTTCTAACGTTTATCTGTC | 3102 |
| mus | GCAAAATGTTTGAAATCCCATTATCAAATGCTAATTTCCACTTTCTAATGTTTATCTGTT | 3226 |
| | ********* ****** ******************* ********** ********** | |
| rattus | GTGGCGTATTAGCCTGTATTTATGTTTGAACAAAGCAGAGGCAACGTGAACCCTCCTCTT | 3162 |
| mus | GTGGCATATTAACCTGTAAT-ATGTTTGAACAAAGCAGAAGTAATATGAACCCTCCTCTT | 3285 |
| | ***** ***** ****** * ****************** * ** ************** | |
| rattus | TTGTAGCCTGCTTTTGCTATCATGGTTTATTTTACAAGTGTTTCTGTTGAATAAACGCAC | 3222 |
| mus | TTGTAGCCTGCTTTTGCTTTCACCGTTGATTTTACAAGTGTTTCTGTTAAATAAACGCAC | 3345 |
| | ****************** *** *** ******************** *********** | |
| rattus | CTTCTACCCTTGTACTGTTACAATAACCCACAGAAAATTTTTAGCTAT------------ | 3270 |
| mus | CTTTTATCCTTGTACTGTTACAATAACCCACAGAAAATTTTTAGCTATCTTTTTCAATTA | 3405 |
| | *** ** ***************************************** | |
| rattus | ----------------- | |
| mus | AAACCAATGCAATTGTT | 3422 |
| ErbB4 | | |
| rattus | ------------------------------------------------------------ | |
| mus | ACTCCGGGAACTAGCTGTACGTTGTGCTCGGAGCACCAGCCGCACAGTCGCGCTCACTCC | 60 |
| rattus | ------------------------------------------------------------ | |
| mus | CACCCGCGCGCCCTCCTCCGCGGCCCCTTGCCGGGTCCGCGGGTCCACGGGTCCTGGAAG | 120 |
| rattus | ------------------------------------------------------------ | |
| mus | CCGCCGCCGTCGCCGACTGGCTCTCCGGCCCCGGGAAGCCCGTGCACCAAGCGCGCCGCG | 180 |
| rattus | ------------------------------------------------------------ | |
| mus | CCCGCCCCCCTTGCGCCCCCCACGCGCTCCCGGCTGAGGGGGGGAGATCTCCTCCGCGTG | 240 |
| rattus | ------------------------------AATTGTCAGCACGAATTCTGAGACTTGCCA | 30 |
| mus | CTCGCAAGTGGCTATGGTATTTGGACATGTAATTGTCAGCGCGGGATCTGAGACTTGCCA | 300 |
| | ********** ** ************** | |
| rattus | AAAATGAAGCTGGCGACGGGACTGTGGGTCTGGGGGAGCCTTCTGGTGGCAGCCAGGACC | 90 |
| mus | AAAATGAAGCTGGCGACGGGACTCTGGGTCTGGGGGAGCCTTCTGATGGCAGCGGGGACC | 360 |
| | *********************** ********************* ******* ***** | |
| rattus | GTCCAGCCCAGCGCTTCTCAGTCAGTGTGTGCCGGAACAGAGAACAAACTGAGCTCTCTC | 150 |
| mus | GTCCAGCCCAGCGCTTCTCAGTCAGTGTGCGCAGGAACAGAGAACAAACTGAGCTCTCTC | 420 |
| | ***************************** ** *************************** | |
| rattus | TCTGATCTGGAGCAGCAGTACCGAGCCTTGCGCAAATACTATGAAAACTGCGAGGTAGTC | 210 |
| mus | TCTGACCTGGAACAGCAGTACCGAGCCTTGCGCAAATACTATGAAAACTGCGAGGTAGTC | 480 |
| | ***** ***** ************************************************ | |
| rattus | ATGGGCAACCTGGAGATCACCAGCATAGAGCACAACCGGGACCTCTCCTTCCTGCGGTCT | 270 |
| mus | ATGGGCAACCTGGAGATCACCAGCATCGAGCACAACCGGGACCTCTCCTTCCTGCGGTCT | 540 |
| | ************************** ********************************* | |
| rattus | ATCCGAGAAGTCACAGGCTATGTACTTGTGGCCCTCAACCAGTTTCGTTACCTGCCTCTG | 330 |
| mus | ATCCGAGAAGTCACAGGCTACGTCCTGGTGGCCCTCAACCAGTTTCGTTACTTGCCTCTG | 600 |
| | ******************** ** ** ************************ ******** | |
| rattus | GAGAATTTACGCATTATTCGTGGGACAAAACTGTATGAAGATCGCTATGCCTTAGCAATA | 390 |
| mus | GAGAATTTACGCATTATTCGTGGGACAAAACTATATGAAGATCGCTATGCCTTAGCGATA | 660 |
| | ******************************** *********************** *** | |
| rattus | TTCTTAAACTACAGGAAAGATGGCAACTTTGGACTTCAAGAACTGGGATTAAAGAACCTG | 450 |
| mus | TTCTTAAACTACAGGAAAGATGGCAACTTTGGACTCCAAGAACTTGGATTAAAGAACCTG | 720 |
| | *********************************** ******** *************** | |
| rattus | ACCGAAATACTAAATGGTGGAGTCTATGTAGACCAGAACAAATTCCTATGTTATGCTGAT | 510 |
| mus | ACCGAAATACTAAATGGTGGAGTCTATGTAGACCAGAACAAATTCCTATGTTATGCTGAC | 780 |
| | *********************************************************** | |
| rattus | ACTATACACTGGCAAGATATTGTTCGGAATCCATGGCCTTCCAACATGACTCTGGTGTCA | 570 |
| mus | ACTATACACTGGCAAGATATTGTTCGGAATCCATGGCCTTCCAACATGACTCTGGTGTCA | 840 |
| | ************************************************************ | |
| rattus | ACAATTGGAAGTTCTGGATGCGGAAGATGCCATAAGTCTTGCACTGGTCGATGCTGGGGA | 630 |
| mus | ACAAATGGAAGTTCTGGATGTGGAAGATGCCATAAGTCTTGCACTGGCCGATGCTGGGGA | 900 |
| | **** *************** ************************** ************ | |
| rattus | CCCACAGAAAATCACTGCCAGACCTTGACAAGGACTGTGTGTGCAGAACAATGTGATGGC | 690 |
| mus | CCCACAGAAAATCACTGCCAGACCTTGACCAGAACTGTGTGTGCTGAACAATGTGATGGC | 960 |
| | ***************************** ** *********** *************** | |
| rattus | AGGTGCTATGGACCCTACGTCAGTGACTGCTGCCATCGAGAATGTGCCGGAGGCTGCTCA | 750 |
| mus | AGGTGCTATGGACCCTACGTTAGTGACTGCTGCCATCGAGAATGTGCTGGAGGCTGCTCA | 1020 |
| | ******************** ************************** ************ | |
| rattus | GGACCAAAAGACACTGACTGCTTTGCCTGCATGAACTTCAATGACAGTGGAGCATGTGTT | 810 |
| mus | GGACCAAAGGACACTGACTGCTTTGCCTGCATGAACTTCAATGACAGTGGAGCCTGCGTT | 1080 |
| | ******** ******************************************** ** *** | |
| rattus | ACTCAGTGTCCCCAAACGTTCGTCTACAATCCAACCACCTTTCAACTGGAACACAACTTC | 870 |
| mus | ACTCAATGTCCCCAAACATTTGTCTACAATCCAACCACCTTTCAACTGGAACACAACTTC | 1140 |
| | ***** *********** ** *************************************** | |
| rattus | AATGCAAAGTACACATATGGAGCATTCTGTGTTAAGAAATGTCCACATAACTTCGTGGTA | 930 |
| mus | AATGCAAAGTACACGTATGGAGCATTCTGTGTTAAGAAATGTCCACATAACTTCGTGGTA | 1200 |
| | ************** ********************************************* | |
| rattus | GATTCCAGTTCTTGTGTACGAGCCTGCCCTAGTTCCAAGATGGAAGTCGAAGAAAATGGA | 990 |
| mus | GATTCCAGTTCTTGTGTACGAGCCTGCCCTAGTTCTAAGATGGAAGTACAAGAAAATGGG | 1260 |
| | *********************************** *********** *********** | |
| rattus | ATTAAAATGTGTAAGCCTTGCACTGATATTTGCCCCAAAGCATGTGATGGAATCGGCACC | 1050 |
| mus | ATTAAAATGTGTAAGCCTTGCACCGATATTTGCCCCAAAGCATGTGATGGAATCGGCACG | 1320 |
| | *********************** *********************************** | |
| rattus | GGATCCTTGATGTCTGCTCAGACTGTGGATTCCAGTAACATTGACAAATTCATAAACTGC | 1110 |
| mus | GGATCACTGATGTCTGCTCAGACTGTGGATTCAAGTAACATTGACAAATTCATAAACTGC | 1380 |
| | ***** ************************* *************************** | |
| rattus | ACCAAGATCAACGGGAATCTCATCTTTCTTGTCACTGGCATTCATGGGGACCCTTACAAT | 1170 |
| mus | ACAAAGATCAATGGCAATCTCATCTTTCTTGTCACTGGCATTCATGGAGACCCTTACAAT | 1440 |
| | ** ******** ** ******************************** ************ | |
| rattus | GCTATTGACGCCATAGACCCAGAGAAACTGAATGTCTTTCGGACAGTCAGAGAAATAACA | 1230 |
| mus | GCTATTGACGCCATAGATCCAGAGAAACTGAATGTCTTTCGGACTGTCAGAGAAATAACA | 1500 |
| | ***************** ************************** *************** | |
| rattus | GGTTTCCTGAACATACAGACTTGGCCCCCAAATATGACAGATTTCAGTGTTTTCTCCAAC | 1290 |
| mus | GGTTTCCTGAACATACAGTCTTGGCCCCCAAATATGACAGATTTCAGTGTTTTCTCCAAC | 1560 |
| | ****************** ***************************************** | |
| rattus | CTTGTAACCATTGGAGGAAGAGTCCTCTACAGTGGTCTGTCATTGCTTATCCTCAAACAA | 1350 |
| mus | CTCGTCACAATTGGAGGAAGAGTCCTCTACAGTGGTCTCTCATTGCTGATCCTCAAACAA | 1620 |
| | ** ** ** ***************************** ******** ************ | |
| rattus | CAAGGTATCACTTCACTACAGTTCCAGTCTCTGAAGGAAATCAGTGCGGGCAATATCTAC | 1410 |
| mus | CAAGGTATCACTTCCCTAGAGTTCCAGTCTCTGAAGGAAATCAGTGCGGGCAATATCTAC | 1680 |
| | ************** ********************************************* | |
| rattus | ATCACGGACAACAGCAACCTGTGTTATTACCACACCATCAACTGGACAACACTGTTCAGC | 1470 |
| mus | ATCACTGACAACAGCAACCTGTGTTATTACCATACCATTAACTGGACAACACTCTTCAGC | 1740 |
| | ***** ************************** ***** ************** ****** | |
| rattus | ACCGTTAACCAGAGGATAGTGATCCGAGACAACAGGAGGGCTGAGAACTGTACTGCTGAA | 1530 |
| mus | ACCATTAACCAGAGAATAGTGATCCGAGATAACAGAAGAGCTGAGAATTGTACTGCTGAA | 1800 |
| | *** ********** ************** ***** ** ******** ************ | |
| rattus | GGGATGGTGTGTAACCACCTGTGTTCAAATGATGGTTGTTGGGGACCTGGGCCAGACCAG | 1590 |
| mus | GGCATGGTATGCAACCACCTGTGTTCAAATGATGGTTGTTGGGGACCTGGGCCGGACCAG | 1860 |
| | ** ***** ** ***************************************** ****** | |
| rattus | TGTCTGTCATGTCGGCGCTTCAGCAGGGGAAAGATCTGTATAGAGTCCTGCAACCTTTAT | 1650 |
| mus | TGCCTGTCATGTCGGCGCTTCAGCAGGGGAAAGATCTGCATAGAGTCTTGCAACCTTTAT | 1920 |
| | ** *********************************** ******** ************ | |
| rattus | GATGGGGAGTTTCGAGAGTTTGAAAATGGCTCCATCTGTGTTGAGTGTGACTCCCAGTGT | 1710 |
| mus | GATGGGGAATTTCGAGAGTTTGAAAACGGCTCCATCTGTGTTGAGTGTGACTCCCAGTGT | 1980 |
| | ******** ***************** ********************************* | |
| rattus | GAGAAAATGGAAGACGGACTCCTCACATGCCATGGACCGGGACCTGACAACTGTACAAAG | 1770 |
| mus | GAGAAAATGGAAGATGGACTCCTCACATGCCATGGACCGGGACCTGACAACTGCACAAAG | 2040 |
| | ************** ************************************** ****** | |
| rattus | TGTTCTCATTTTAAAGATGGTCCAAACTGTGTAGAGAAATGTCCAGATGTCCTACAGGGA | 1830 |
| mus | TGCTCTCATTTTAAGGATGGTCCAAACTGTGTGGAGAAATGTCCAGATGGCCTACAGGGA | 2100 |
| | ** *********** ***************** **************** ********** | |
| rattus | GCAAACAGTTTCATATTTAAGTACGCAGATCAGGATCGGGAGTGCCACCCTTGCCATCCA | 1890 |
| mus | GCAAACAGTTTCATTTTTAAGTATGCAGATCAGGATCGGGAGTGCCACCCTTGCCATCCA | 2160 |
| | ************** ******** ************************************ | |
| rattus | AACTGCACCCAGGGGTGTAACGGTCCCACTAGTCATGACTGCATTTACTACCCATGGACG | 1950 |
| mus | AACTGCACCCAGGGGTGTAACGGTCCCACTAGTCATGACTGCATTTACTACCCATGGACG | 2220 |
| | ************************************************************ | |
| rattus | GGCCATTCCAGTTTACCACAACATGCTAGAACTCCACTGATTGCAGCCGGAGTCATTGGT | 2010 |
| mus | GGCCATTCCACTTTACCACAACACGCTAGAACTCCACTGATTGCAGCCGGAGTCATTGGA | 2280 |
| | *********************** *********************************** | |
| rattus | GGGCTCTTCATCCTGGTCATCATGGCTCTGACATTTGCCGTTTATGTCAGAAGGAAGAGC | 2070 |
| mus | GGCCTCTTCATCCTGGTGATCATGGCTTTGACATTTGCTGTCTATGTCAGAAGAAAGAGC | 2340 |
| | ** ************** ********* ********** ** *********** ****** | |
| rattus | ATCAAAAAGAAACGCGCTTTGAGAAGATTCCTGGAGACCGAGTTAGTCGAGCCCTTAACC | 2130 |
| mus | ATCAAAAAGAAACGTGCTTTGAGGAGATTCCTGGAGACAGAGCTGGTAGAGCCCTTAACT | 2400 |
| | ************** ******** ************** *** * ** *********** | |
| rattus | CCTAGTGGCACAGCACCCAATCAAGCTCAACTTCGAATTTTGAAGGAAACAGAGCTAAAG | 2190 |
| mus | CCCAGTGGCACGGCACCCAATCAAGCTCAACTTCGCATTTTGAAGGAAACCGAACTAAAG | 2460 |
| | ** ******** *********************** ************** ** ****** | |
| rattus | AGGGTAAAAGTCCTTGGCTCGGGAGCATTTGGAACCGTTTATAAAGGAATCTGGGTACCT | 2250 |
| mus | AGGGTAAAGGTCCTTGGCTCGGGAGCTTTTGGAACCGTTTATAAAGGTATTTGGGTGCCT | 2520 |
| | ******** ***************** ******************** ** ***** *** | |
| rattus | GAAGGAGAAACTGTGAAAATCCCTGTGGCTATTAAGATCCTCAATGAGACAACTGGCCCC | 2310 |
| mus | GAAGGTGAAACAGTGAAAATCCCTGTGGCTATAAAGATCCTCAATGAAACAACTGGCCCC | 2580 |
| | ***** ***** ******************** ************** ************ | |
| rattus | AAAGCCAATGTGGAGTTCATGGATGAGGCACTGATTATGGCAAGTGTGGATCACCCACAC | 2370 |
| mus | AAAGCCAACGTGGAGTTCATGGATGAGGCTCTGATCATGGCAAGTATGGATCACCCACAC | 2640 |
| | ******** ******************** ***** ********* ************** | |
| rattus | CTAGTGCGTTTACTGGGTGTGTGTTTGAGCCCCACTATCCAGTTGGTTACTCAGTTGATG | 2430 |
| mus | CTAGTTCGCCTATTGGGAGTGTGTCTGAGTCCCACTATCCAGTTGGTTACGCAGCTGATG | 2700 |
| | ***** ** ** **** ****** **** ******************** *** ***** | |
| rattus | CCACATGGCTGCCTACTGGAATATGTCCACGAACACAAGGATAACATCGGATCACAACTG | 2490 |
| mus | CCGCATGGCTGCCTACTGGACTATGTTCATGAACACAAGGATAACATTGGATCACAGCTG | 2760 |
| | ** ***************** ***** ** ***************** ******** *** | |
| rattus | CTGTTGAACTGGTGTGTCCAGATTGCTAAGGGAATGATGTATCTGGAGGAAAGGCGGCTT | 2550 |
| mus | CTGTTGAACTGGTGTGTCCAGATTGCTAAGGGAATGATGTACCTAGAAGAAAGACGGCTT | 2820 |
| | ***************************************** ** ** ***** ****** | |
| rattus | GTTCATCGGGATCTGGCAGCCCGCAATGTGTTGGTGAAATCTCCAAATCATGTTAAAATC | 2610 |
| mus | GTTCATCGGGATCTGGCAGCCCGCAATGTCTTAGTGAAATCTCCAAATCATGTTAAAATC | 2880 |
| | ***************************** ** *************************** | |
| rattus | ACAGACTTTGGACTGGCCCGGCTCTTGGAAGGAGATGAAAAAGAATACAATGCTGACGGT | 2670 |
| mus | ACAGATTTTGGACTGGCCCGGCTCTTGGAAGGAGATGAAAAAGAATACAATGCTGATGGT | 2940 |
| | ***** ************************************************** *** | |
| rattus | GGCAAGATGCCAATTAAATGGATGGCTCTGGAATGTATACATTATAGGAAATTCACACAT | 2730 |
| mus | GGCAAGATGCCAATTAAATGGATGGCTCTGGAATGTATACATTATAGGAAATTCACACAT | 3000 |
| | ************************************************************ | |
| rattus | CAAAGCGATGTTTGGAGCTACCGTGTCACTATATGGGAACTGATGACCTTTGGAGGAAAG | 2790 |
| mus | CAAAGTGATGTTTGGAGCTATGGCGTCACTATATGGGAACTGATGACCTTTGGAGGAAAG | 3060 |
| | ***** ************** ** ************************************ | |
| rattus | CCCTATGATGGAATTCCAACGCGAGAAATCCCTGATTTATTAGAGAAGGGAGAGCGTTTG | 2850 |
| mus | CCCTATGATGGAATTCCAACCCGAGAAATCCCCGATTTACTGGAGAAAGGAGAGCGTCTC | 3120 |
| | ******************** *********** ****** * ***** ********* ** | |
| rattus | CCTCAACCTCCCATCTGCACTATTGACGTTTACATCGTCATGGTCAAATGTTGGATGATC | 2910 |
| mus | CCTCAGCCTCCCATCTGCACTATTGATGTTTACATGGTCATGGTCAAATGTTGGATGATC | 3180 |
| | ***** ******************** ******** ************************ | |
| rattus | GATGCTGACAGCAGACCTAAATTCAAAGAACTGGCTGCTGAGTTTTCAAGGATGGCTAGA | 2970 |
| mus | GATGCTGACAGCAGACCTAAATTCAAAGAACTGGCTGCTGAGTTTTCAAGAATGGCTAGA | 3240 |
| | ************************************************** ********* | |
| rattus | GACCCTCAAAGATACCTAGTAATTCAGGGGGATGATCGCATGAAGCTTCCCAGTCCAAAC | 3030 |
| mus | GACCCTCAAAGATACCTAGTTATTCAGGGTGATGATCGTATGAAGCTTCCCAGTCCAAAT | 3300 |
| | ******************** ******** ******** ******************** | |
| rattus | GACAGCAAATTCTTCCAGAATCTCTTGGATGAAGAGGATTTGGAAGATATGATGGACGCT | 3090 |
| mus | GACAGCAAATTCTTCCAGAATCTCTTGGATGAAGAGGATTTGGAAGACATGATGGATGCT | 3360 |
| | *********************************************** ******** *** | |
| rattus | GAGGAATATTTGGTCCCCCAGGCTTTCAATATCCCACCTCCTATCTACACATCCAGAACA | 3150 |
| mus | GAGGAATATTTGGTCCCCCAGGCTTTCAACATACCTCCTCCCATCTACACATCCAGAACA | 3420 |
| | ***************************** ** ** ***** ****************** | |
| rattus | AGAATTGACTCCAATAGGAGTGAAATTGGACACAGCGCTCCTCCTGCCTACACCCCCATG | 3210 |
| mus | AGAATTGACTCCAATAGGAAT--------------------------------------- | 3441 |
| | ******************* * | |
| rattus | TCGGGAAGTCAGTTTGTGTACCAGGATGGGGGTTTCGCTACACAACAAGGAATGCCCATG | 3270 |
| mus | ---------CAGTTTGTGTACCAAGATGGGGGCTTTGCTACACAACAAGGAATGCCCATG | 3492 |
| | ************** ******** ** ************************ | |
| rattus | CCCTACACAGCCACAACCAGCACCATACCAGAGGCTCCAGTCGCCCAGGGTGCAACGGCT | 3330 |
| mus | CCCTACAGAGCCACAACCAGCACCATACCAGAGGCTCCAGTAGCTCAGGGTGCAACGGCT | 3552 |
| | ******* ********************************* ** *************** | |
| rattus | GAGATGTTTGATGACTCCTGCTGTAATGGTACCCTGCGAAAGCCAGTGGTACCCCACGTC | 3390 |
| mus | GAGATGTTTGATGACTCCTGCTGTAATGGTACCCTACGAAAGCCAGTGGCACCCCATGTC | 3612 |
| | *********************************** ************* ****** *** | |
| rattus | CAAGAGGACAGTAGCACTCAGAGGTATAGTGCCGACCCCACAGTGTTCGCCCCAGAACGG | 3450 |
| mus | CAAGAGGACAGTAGCACTCAGAGGTATAGTGCTGATCCCACAGTGTTCGCCCCAGAACGG | 3672 |
| | ******************************** ** ************************ | |
| rattus | AACCCACGAGGAGAACTGGATCAAGAAGGCTACATGACTCCCATGCATGACAAGCCAAAA | 3510 |
| mus | AATCCTCGAGGAGAACTGGATGAAGAAGGCTACATGACTCCAATGCATGACAAGCCCAAA | 3732 |
| | ** ** **** ****************************** ************** *** | |
| rattus | CAAGAATATCTGAATCCTGTGGAAGAGAACCCTTTTGTGTCCCGGAGGAAGAATGGAGAC | 3570 |
| mus | CAAGAATATCTGAATCCTGTGGAAGAGAACCCTTTTGTGTCCCGAAGGAAGAATGGAGAT | 3792 |
| | ******************************************** ************** | |
| rattus | CTTCAAGCTTTAGATAATCCAGAGTATCACAGCGCTTCCAGCGGTCCCCCCAAGGCAGAG | 3630 |
| mus | CTTCAAGCTTTAGATAATCCGGAGTATCACAGTGCTTCCAGCGGTCCACCCAAGGCGGAG | 3852 |
| | ******************** *********** ************** ******** *** | |
| rattus | GATGAGTACGTGAATGAGCCCCTTTATCTCAACACCTTCACCAACGCCTTGGGAAATGCA | 3690 |
| mus | GATGAATACGTGAATGAGCCTCTATACCTCAACACCTTCGCCAATGCCTTGGGGAGTGCA | 3912 |
| | ***** ************** ** ** ************ **** ******** * **** | |
| rattus | GAGTACATGAAAAACAGCTTACTGTCTGTGCCAGAGAAAGCCAAGAAAGCATTTGACAAC | 3750 |
| mus | GAGTACATGAAAAACAGTGTACTGTCTGTGCCAGAGAAAGCCAAGAAAGCATTTGACAAC | 3972 |
| | ***************** ***************************************** | |
| rattus | CCCCACTACTGGAACCACAGCCTGCCACCCCGGAGCACTCTTCAGCACCCAGACTACCTG | 3810 |
| mus | CCCGACTACTGGAACCACAGCCTGCCACCCCGGAGCACCCTTCAGCACCCAGACTACCTG | 4032 |
| | ************************************** ********************* | |
| rattus | CAGGAATACAGCACAAAATATTTTTATAAACAGAATGGACGGATCCGCCCTATTGTGGCA | 3870 |
| mus | CAGGAATACAGCACAAAATATTTTTATAAACAGAATGGACGGATCCGCCCCATTGTGGCA | 4092 |
| | ************************************************** ********* | |
| rattus | GAGAATCCTGAGTACCTCTCAGAGTTCTCGCTGAAGCCAGGCACTATGCTGCCCCCTCCG | 3930 |
| mus | GAGAATCCTGAGTACCTCTCGGAGTTCTCGCTGAAGCCTGGCACTATGCTGCCCCCTCCG | 4152 |
| | ******************** ***************** ********************* | |
| rattus | CCCTACAGACACCGGAATACTGTGGTGTGAGCTCAGCTAGAGTGTTTTAGGAGCAGAAAC | 3990 |
| mus | CCCTACAGACACCGGAATACTGTGGTGTGAGCTTGGCTAGAGTGTTAGGTGGAGAAACAC | 4212 |
| | ********************************* *********** * * * ** | |
| rattus | ACAGCCGCTCCATTTCCCCTTCTCCCTCCTCTTTCTCTGGCAGTCTTCCTTCTACCCCAA | 4050 |
| mus | ACACCCACTCCATTTCCC-TTCCCCCTCCTCTTTCTCTGGTGGTCT-------------- | 4257 |
| | ****** *********** *** ***************** **** | |
| rattus | GGCCAGTAGT | 4060 |
| mus | ---------- | |

Pain Gene Knockout Phenotypes.

Neuregulin-1. (Nrg1) Knockout, complete loss of function phenotype.

The interaction between peripheral axons and myelinating Schwann cells is dependent on Nrg1 expression. In order to study and develop animal models for pain Nrg1 knockout rats were created by transposon mediated insertion. Genetic modification to Rattus norvegicus pain gene Neuregulin-1 (Nrg1) was carried out by a DNA transposon insertional mutagenesis method similar to that described in Nature Genet., 25, 35 (2000). The DNA transposon-mediated genetically modified allele was designated Nrg1 Tn(sb-T2/Bart3)2.183Mcwi. The mutant strain symbol for the pain rat was designated F344-Nrg1 Tn(sb-T2/Bart3)2.183Mcwi. The DNA transposon insertion occurred in chromosome 16, within intron 1 of the rat Nrg1 gene. The sequence tag map position was between base pairs: 174755561 174756178. The sequence tag was:

TACATATACATATACATATACATATACATATACATATACATA TACATATACATATACATATACATATACATATACATCATATAC ATATACCCAGAGAGAGGGAGATAGTGCATATACATATAGTG TTTTTATCAATTGATTACAATTTCATAATTATCCTTATTCACA AAGTCATGCATTATGACTATATTCACTTTCCATTCCTCCTCCA AAACCTCCCAGCTCCAGTCCTACCCCCTAACTTGCTCTCAAT TTCATGTCTGCTTTTGTTTCCTTATCACTATAAACCACCAAGT CAGCTTCTACTCCTAG. Thus, a DNA transposon was inserted into the Nrg1 gene of Rattus norvegicus and Western blot analysis indicated that the gene was completely inactive. Since Nrg1 plays an important role in Schwann cell development and myelination it was suspected that the axon mediator was involved in pain. To induce pain spinal nerve ligation (SNL) surgery was done on both Nrg1 (−/−) and WT rats. Tight ligation of spinal nerves by surgery created groups of control and KO pain animals. A baseline threshold for mechanical pain was established in order to eliminate startle reactions. Animals were placed in an elevated wire mesh floor. Mechanical allodynia/hyperalgesia was assessed by utilizing multiple von Frey filaments of different forces. In ascending order of force the filaments were applied to the hind paw of control and Nrg1 knockout rats. Withdrawal responses were recorded within 5s of filament application and an overall response percentage was calculated. When the Nrg1 knockout rat response to mechanical pain was calculated the model showed an almost absent response compared to the WT controls. The Nrg1 (−/−) rat had a 10% vehicle response rate when compared to the WT control animals. This study validated the Nrg1 knockout rat as a genetically modified animal model for pain. To supplement the mechanical pain data a cold behavioral test was also done using acetone. In the same mesh floor box a drop of acetone was placed on the center of the ventral side of control and Nrg1 (−/−) knockout rat hindpaws. For 20s after the acetone treatment the rat's response was recorded. Induced pain by nerve ligation, disease state, and drug exposure produces significant cold allodynia. Acetone induced cold-allodynia pain responses include: quick withdrawal, flick or stamp, prolonged withdrawal with multiple flicking, and licking or biting of the affected hindpaw. These responses were added up to produce a cold sensitivity score. The average cold score for WT control rats after acetone treatment was 8. The average cold-score of Nrg1 knockout rats following acetone treatment was 0.1. This study indicated that the Nrg1 knockout rats were close to completely deficient in cold allodynia pain response. These data exhibit that the Nrg1 knockout rat displays a decreased sensitivity to multiple forms of pain. The data explains and validates a knockout rat model for pain.

Transient Receptor Potential (TRP) Channel 4 (Trpc4) Knockout, Complete Loss of Expression Phenotype.

Transient receptor potential (TRP) channels are essential for transmembrane Ca2+ and Na+ flux, axon guidance and neurite extension. Due to TRY involvement in the nervous system Trpc4 knockout rats were created by transposon insertional mutagenesis. Genetic modification to Rattus norvegicus pain gene Transient receptor potential channel 4 (Trpc4) was carried out by a DNA transposon insertional mutagenesis method similar to that described in Nature Genet., 25, 35 (2000). The DNA transposon-mediated genetically modified allele was designated Trpc4Tn(sb-T2/Bart3)2.192Mcwi. The mutant strain symbol for the pain rat was designated F344-Trpc4Tn(sb-T2/Bart3)2.192Mcwi. The DNA transposon insertion occurred in chromosome 2, within intron 1 of the rat Trpc4 gene. The sequence tag map position was between base pairs: 143344742-143344909. The sequence tag was: TATGTTTAGGCCATGGAGATAAGAGGCATCTTCCAGAGTTA GGAATTACATACATCTGCACTTATGTATCACGATTATGCTTC TGAATGCACCTAACAAGAGCTCGAGGAGAAACCATGCAGAG AGGAACAATTGAAAAGGAAGTACATTGTGCAGACTGCTTCC TAG. Thus, a DNA transposon was inserted into the Trpc4 gene of Rattus norvegicus and Western blot analysis indicated that the gene was completely inactive. To study the effect of Trpc4 on pain two mechanical pain tests, diabetes induced, and drug induced studies were done on the animal models. First the spared nerve injury (SNI) operation was performed on a set of controls and Trpc4 knockout rats. Once the sciatic nerve and terminal branches were exposed on the lateral side of the back paw the peroneal and tibial nerves were ligated. Groups of wildtype and Trpc4 knockout rats also underwent partial nerve injury (PNI) by tightening the siactic nerve of approximately ⅓-½ of the normally functional diameter. From the SM and PNI pain induced rats, controls and Trpc4 knockouts were assessed for response to mechanical pain via von Frey filaments as described above. It was observed that under both pain models Trpc4 knockout rats exhibited 10-20% of the pain response that was displayed in wildtype control rats. This mechanical test indicated that Trpc4 knockout rats were indeed models for pain.

Since many patients with diabetes mellitus also suffer from hyperalgesia derived pain the Trpc4(−/−) rats were studied for this indication. Groups of control and Trpc4 knockout rats were treated with streptozotocin (STZ) in order to induce diabetes. Rats were observed to be in a diabetic state if the presence of hyperglycemia, and glucosuria occurred. The mechanical induced von Frey filament method was again used to establish altered pain response. When STZ non-treated control rats were compared to STZ diabetic control rats the pain response to mechanical induction was very drastic. The control non-treated rats maintained the threshold as expected. However, the STZ treated diabetic rats threshold for pain was decreased by 40%. Therefore, the STZ treated control rats were 40% more sensitive to mechanical induced pain. When control STZ induced control and Trpc4−/− rats were compared for mechanical induced sensitivity to pain the change was significant. The STZ treated diabetic Trpc4−/− rats exhibited a similar threshold for pain as the control non-treated rats. Therefore, the Trpc4−/− rats were able to recover the STZ induced diabetic neuropathy phenotype. These data prove that the Trpc4 knockout rat is a model for diabetic induced pain.

Cancer patients who are treated with paclitaxel often display sensory abnormalities and symptoms of pain such as sudden unexplainable pain attacks. In order to study the effects of cation channels on pacitaxel induced pain Trpc4−/− rats were treated with the anti-cancer drug. Since one of the major symptoms of patients who are treated with pacitaxel is cold allodynia control and Trpc4−/− rats were exposed to pacitaxel and tested for acetone cold response. Previously control non-pacitaxel treated and pacitaxel treated WT rats were studied for differences in cold allodynia. Based on established cold score calculations the non-treated rats scored an average of 8 while the pacitaxel treated rats scored an average of 15. These data indicate that pacitaxel treated rats are indeed hypersensitive to cold induced pain. When control and Trpc4−/− treated rats were compared the difference in pain sensitivity was dramatic. Trpc4 knockout rats which were treated with pacitaxel displayed a cold score of 9. The recovery of a nearly wild type cold allodynia score was remarkable in Trpc4 knockout rats. These data implicate Trpc4 as a key mediator of drug (pacitaxel) induced pain.

ErbB-signaling, v-erb-a erythroblastic leukemia viral oncogene homolog 4 (ErbB4) knockout, complete loss of expression phenotype.

ErbB signaling for the interaction of myelination in axons and Schwann cells has been implicated in sensory disorders as a result of C-fiber and Schwann cell apoptosis. In order to study this phenomenon transposon mediated mutagenesis was done to generate v-erb-a erythroblastic leukemia viral oncogene homolog 4 (ErbB4) knockout rats. Genetic modification to Rattus norvegicus ErbB4 was carried out by a DNA transposon insertional mutagenesis method similar to that described in Nature Genet., 25, 35 (2000). The DNA transposon-mediated genetically modified allele was designated Erbb4Tn(sb-T2/Bart3)2.208Mcwi. The mutant strain symbol for the pain rat was designated F344-Erbb4Tn(sb-T2/Bart3)2.208Mcwi. The DNA transposon insertion occurred in chromosome 9, within intron 1 of the rat ErbB4 gene. The sequence tag map position was between base pairs: 67440981-67441017. The sequence tag was: TACATCCATGTTTTTCTACTGATGTCCTTGTCTCTAG. Thus, a DNA transposon was inserted into the ErbB4 gene of Rattus norvegicus and Western blot analysis indicated that the gene was completely inactive. When the sciatic nerves of 40 day old ErbB4−/− rats were examined it had a diameter that was nearly 50% smaller than the width of a wild type nerve. In order to study a sensory defect phenotype the rats were placed in hot and cold plates set on 55 C and −5 C respectively. Interestingly, knockout rats exhibited a progressive hot and cold sensory defect. At the age of 3 weeks Erbb4 knockout rats responded similar to WT when tested for paw withdrawal latency to the hot and cold plates. At 3 weeks of age the rats paw withdrawal was almost even at around 10 seconds of exposure to heat plate. However, at the age of 6 weeks the ErbB4−/− rat paw withdrawals of over 30 seconds compared to a relatively unchanged WT withdrawal. This phenotype displays the clear development of a sensory defect to thermal heat in ErbB−/− rats. A similar result was obtained using the cold plate. By 5 weeks of age all homozygous ErbB4 knockout rats exhibit this sensory defect. These data validate the ErbB4 knockout rat model as a pain animal model.

### Examples

The rat and progenies thereof of the present invention may be any rat or progenies thereof, so long as they are a rat or progenies thereof in which genome is modified so as to have decreased or deleted activity of the pain gene.

Gene Disruption Technique which Targets at a Gene Encoding Neuregulin-1 (Nrg1) and Transient receptor potential family 4 (Trpc4).

The gene disruption method may be any method, so long as it can disrupt the gene of the target enzyme. Examples include a homologous recombination method, a method using retrovirus, a method using DNA transposon, and the like.

(a) Preparation of the Rat and Progenies Thereof of the Present Invention by Homologous Recombination

The rat and the progenies thereof of the present invention can be produced by modifying a target gene on chromosome through a homologous recombination technique which targets at a gene encoding the pain gene. The target gene on chromosome can be modified by using a method described in Gene Targeting, A Practical Approach, IRL Press at Oxford University Press (1993) (hereinafter referred to as “Gene Targeting, A Practical Approach”); or the like, for example.

Based on the nucleotide sequence of the genomic DNA, a target vector is prepared for homologous recombination of a target gene to be modified (e.g., structural gene of the pain gene, or a promoter gene). The prepared target vector is introduced into an embryonic stem cell and a cell in which homologous recombination occurred between the target gene and target vector is selected.

The selected embryonic stem cell is introduced into a fertilized egg according to a known injection chimera method or aggregation chimera method, and the embryonic stem cell-introduced fertilized egg is transplanted into an oviduct or uterus of a pseudopregnant female rat to thereby select germ line chimeras.

The selected germ line chimeras are crossed, and individuals having a chromosome into which the introduced target vector is integrated by homologous recombination with a gene region on the genome which encodes the pain protein are selected from the born offsprings.

The selected individuals are crossed, and homozygotes having a chromosome into which the introduced target vector is integrated by homologous recombination with a gene region on the genome which encodes the pain protein in both homologous chromosomes are selected from the born offsprings. The obtained homozygotes are crossed to obtain offspring to thereby prepare the rat and progenies thereof of the present invention.

(b) Preparation of the Rat and Progenies Thereof of the Present Invention by a Method Using a Transposon

The rat and progenies thereof of the present invention can be prepared by using a transposon system similar to that described in Nature Genet., 25, 35 (2000) or the like, and then by selecting a mutant of the pain gene.

The transposon system is a system in which a mutation is induced by randomly inserting an exogenous gene into chromosome, wherein an gene trap cassette or exogenous gene interposed between transposons is generally used as a vector for inducing a mutation, and a transposase expression vector for randomly inserting the gene into chromosome is introduced into the cell at the same time. Any transposase can be used, so long as it is suitable for the sequence of the transposon to be used. As the gene trap cassette or exogenous gene, any gene can be used, so long as it can induce a mutation in the DNA of the cell.

The rat and progenies thereof of the present invention can be prepared by introducing a mutation into a gene encoding the pain associated protein, and then by selecting a rat of interest in which the DNA is mutated.

Specifically, the method includes a method in which a rat of interest in which the mutation occurred in the gene encoding the NRG1, TRPC4, ERBB4 protein is selected from mutants born from generative cells which are subjected to mutation-inducing treatment or spontaneously generated mutants. In another embodiment, the pain gene is one of several known pain genes, such as (Pparα, Pparγ, Trpml3, Trpml6, Trpm8, Trpv1, Trpa1, Trpc3, Trpc5, Scn9a, Ntrk1, Wnk1, Hsan1, Sc10a, Hsan3, Ptger2, Pnoc, Gabbr1, Gabbr2, Cacna1g, Tac1, Prx, Homer1, Scn11a, Oprl1, Prlhr, P2x3, Bdkrb1, Ptgs2, Th, Npy1r, P2rx4, Mmp9, Mmp2, Bdnf.) The generative cell includes cells capable of forming an individual such as a sperm, an ovum or a pluripotent cells. The generative cell may also be a somatic cell and the animal may then be created by somatic cell nuclear transfer.

Examples in which several methods described above have been employed by the inventors to create a pain gene model phenotype in Rattus norvegicus are described below. Genetic modification to Rattus norvegicus pain gene Neuregulin-1 (Nrg1) was carried out by a DNA transposon insertional mutagenesis method similar to that described in Nature Genet., 25, 35 (2000). The DNA transposon-mediated genetically modified allele was designated Nrg1Tn(sb-T2/Bart3)2.183Mcwi. The mutant strain symbol for the pain rat was designated F344-Nrg1 Tn(sb-T2/Bart3)2.183Mcwi. The DNA transposon insertion occurred in chromosome 16, within intron 1 of the rat Nrg1 gene. The sequence tag map position was between base pairs: 174755561 174756178. The sequence tag was: TACATATACATATACATATACATATACATATACATATACATA TACATATACATATACATATACATATACATATACATCATATAC ATATACCCAGAGAGAGGGAGATAGTGCATATACATATAGTG TTTTTATCAATTGATTACAATTTCATAATTATCCTTATTCACA AAGTCATGCATTATGACTATATTCACTTTCCATTCCTCCTCCA AAACCTCCCAGCTCCAGTCCTACCCCCTAACTTGCTCTCAAT TTCATGTCTGCTTTTGTTTCCTTATCACTATAAACCACCAAGT CAGCTTCTACTCCTAG. Genetic modification to Rattus norvegicus pain gene Transient receptor potential channel 4 (Trpc4) was carried out by a DNA transposon insertional mutagenesis method similar to that described in Nature Genet., 25, 35 (2000). The DNA transposon-mediated genetically modified allele was designated Trpc4Tn(sb-T2/Bart3)2.192Mcwi. The mutant strain symbol for the pain rat was designated F344-Trpc4Tn(sb-T2/Bart3)2.192Mcwi. The DNA transposon insertion occurred in chromosome 2, within intron 1 of the rat Trpc4 gene. The sequence tag map position was between base pairs: 143344742-143344909. The sequence tag was: TATGTTTAGGCCATGGAGATAAGAGGCATCTTCCAGAGTTA GGAATTACATACATCTGCACTTATGTATCACGATTATGCTTC TGAATGCACCTAACAAGAGCTCGAGGAGAAACCATGCAGAG AGGAACAATTGAAAAGGAAGTACATTGTGCAGACTGCTTCC TAG.

A DNA transposon was inserted into the Nrg1, Trpc4, ErbB4 genes of Rattus norvegicus rendering the gene completely inactive. Neuregulin-1, Transient receptor potential family 4, and V-erb-a erythroblastic leukemia viral oncogene homolog 4 (Nrg1, Trpc4, ErbB4−/−) KO rats exhibited multiple pain phenotypes including hypo- and hyper-sensitiveness to induced pain tests, which included mechanical, cold allodynia, heat, diseases state induction, and drug induced. These rat knockout models are valuable tools for studying pain.

The practice of the present invention will employ, unless otherwise indicated, conventional techniques of molecular biology (including recombinant techniques), microbiology, cell biology and biochemistry, which are within the skill of the art.

1. A genetically modified non-human mammal, or progenies thereof, at least some of whose cells comprise a genome comprising a genetic mutation in one or more genes that causes the mammal to have a greater susceptibility to abnormal condition of pain perception than a mammal not comprising the genetic mutation.

2. The genetically modified nonhuman mammal of claim 1, wherein the mammal is a chimeric mammal.

3. The genetically modified nonhuman mammal of claim 1, wherein the mammal is a rat.

4. The genetically modified nonhuman mammal of claim 3, wherein one or more pain genes or loci are misexpressed.

5. The genetically modified nonhuman mammal of claim 3, wherein one or more pain genes are conditionally misexpressed.

6. The non-human animal model of claim 4, wherein the misexpression results in decreased expression of one or more neuropathic pain gene products.

7. The genetically modified nonhuman mammal of claim 4, wherein the one or more genes encoding a neuropathic pain gene product is disrupted.

8. The genetically modified nonhuman mammal of claim 4, wherein all alleles on the genome of the neuropathic pain gene are disrupted.

9. The genetically modified nonhuman mammal of claim 4, wherein the neuropathic pain gene is selected from the group consisting of Cyp3a4, Nrg1, Trpc4, Trpv1, Trpv3, ErbB4, Pparα, Pparγ, Trpml3, Trpml6, Trpm8, Trpv1, Trpa1, Trpc3, Trpc5, Scn9a, Ntrk1, Wnk1, Hsan1, Sc10a, Hsan3, Ptger2, Pnoc, Gabbr1, Gabbr2, Cacna1g, Tac1, Prx, Homer1, Scn11a, Oprl1, Prlhr, P2x3, Bdkrb1, Ptgs2, Th, Npy1r, P2rx4, Mmp9, Mmp2, and Bdnf.

10. The genetically modified nonhuman mammal of claim 4, wherein the neuropathic pain gene is selected from the group consisting of Cyp3a4, Nrg1, Trpc4, Trpv1, Trpv3 and ErbB.

11. The genetically modified nonhuman mammal of claim 4, wherein Trpc4.

12. The genetically modified nonhuman mammal of claim 4, wherein the cells are somatic cells.

13. The genetically modified nonhuman mammal of claim 4, wherein the cells are hepatocytes.

14. The genetically modified nonhuman mammal of claim 4, wherein the one or more pain genes or loci are disrupted using a method selected from the group consisting of mutating directly in the germ cells of a living organism, removal of DNA encoding all or part of the ion transporter protein, insertion mutation, transposon insertion mutation, deletion mutation, introduction of a cassette or gene trap by recombination, chemical mutagenesis, RNA interference (RNAi), and delivery of a transgene encoding a dominant negative protein, which may alter the expression of a target gene.

15. The genetically modified nonhuman mammal of claim 7, wherein the mammal is homozygous for the one or more disrupted genes or loci.

16. The genetically modified nonhuman mammal of claim 7, wherein the mammal is heterozygous for the one or more disrupted genes or loci.

17. A genetically modified non-human mammal, or progenies thereof, whose genome is disrupted at one or more neuropathic pain gene loci so as to produce a phenotype, relative to a wild-type phenotype, comprising abnormal condition of pain perception of the mammal.

18. The genetically modified nonhuman mammal of claim 16, wherein the disruption causes the mammal to have a greater susceptibility to altered conditions of pain perception.

19. The genetically modified nonhuman mammal of claim 16, wherein the mammal is a rat.

20. The genetically modified nonhuman mammal of claim 16, wherein the disruption causes a complete loss-of-function phenotype.

21. The genetically modified nonhuman mammal of claim 16, wherein the disruption causes a partial loss-of-function phenotype.

22. The genetically modified nonhuman mammal of claim 16, wherein the disruption causes a phenotype resulting from multiple transporter disruptions.

23. The genetically modified nonhuman mammal of claim 16, wherein the protein product of the neuropathic pain gene is associated with the phenotype that is characterized as altered conditions of pain perception.

24. The genetically modified nonhuman mammal of claim 16, wherein the neuropathic pain gene is selected from the group consisting of Cyp3a4, Nrg1, Trpc4, Trpv1, Trpv3 and ErbB.

25. The genetically modified nonhuman mammal of claim 16, wherein Trpc4.

26. The genetically modified nonhuman mammal of claim 16, wherein the one or more pain genes or loci are disrupted by transposon insertion mutations.

27. The genetically modified nonhuman mammal of claim 16, wherein the one or more pain genes or loci are disrupted by deletion mutation.

28. The genetically modified nonhuman mammal of claim 16, wherein the one or more pain genes or loci are disrupted by the introduction of a cassette or gene trap by recombination.

29. The genetically modified nonhuman mammal of claim 16, wherein the one or more pain genes or loci are disrupted by chemical mutagenesis with mutagens.

30. The genetically modified nonhuman mammal of claim 16, wherein the one or more pain genes or loci are disrupted by RNA interference (RNAi).

31. The genetically modified nonhuman mammal of claim 16, wherein the one or more pain genes or loci are disrupted by delivery of a transgene encoding a dominant negative protein, which may alter the expression of a target gene.

32. The genetically modified nonhuman mammal of claim 16, wherein the mammal is homozygous for the one or more disrupted genes or loci.

33. The genetically modified nonhuman mammal of claim 16, wherein the mammal 1 is heterozygous for the one or more disrupted genes or loci.

34. The genetically modified nonhuman mammal of claim 16, wherein the phenotype results from a diminished amount, relative to the wild-type phenotype, of a protein selected from the group consisting of Trpc4.

35-57. (canceled)


SEQUENCE LISTING (17 sequences)

SEQ ID NO: 1 - DNA, 3272 bp, Rattus norvegicus
gcggccgcagctgccgggagatgcgagcgcagaccggattgtgatcacctttccctcttcgggctgtaagagagcgagacaagccaccgaagcgaggccactccagagccggcagcggagggacccgggacactagagcagctccgagccactccagactgagcggacgctccaggtgatcgagtccacgctgcttcctgcaggcgacaggcgacgcctcccgagcagcccggccactggctcttcccctcctgggacaaacttttctgcaagcccttggaccaaacttgtcgcgcgtcaccgtcacccaaccgggtccgcgtagagcgctcatcttcggcgagatgtctgagcgcaaagaaggcagaggcaaggggaagggcaagaagaaggaccggggatcccgcgggaagcccgggcccgccgagggcgacccgagcccagcactgcctcccagattgaaagaaatgaagagccaggagtcagctgcaggctccaagctagtgctccggtgcgaaaccagctccgagtactcctcactcagattcaaatggttcaagaatgggaacgagctgaaccgcaaaaataaaccagaaaacatcaagatacagaagaagccagggaagtcagagcttcgaattaacaaagcatccctggctgactctggagagtatatgtgcaaagtgatcagcaagttaggaaatgacagtgcctctgccaacatcaccattgttgagtcaaacgagttcatcactggcatgccagcctcgactgagacagcctatgtgtcctcagagtctcccattagaatctcagtttcaacagaaggcgcaaacacttcttcatccacatcaacatccacgactgggaccagccatctcataaagtgtgcggagaaggagaaaactttctgtgtgaatgggggcgagtgcttcacggtgaaggacctgtcaaacccgtcaagatacttgtgcaagtgcccaaatgagtttactggtgatcgttgccaaaactacgtaatggccagcttctacaaagcggaggaactctaccagaagagggtgctgacaattactggcatctgtatcgccctgctggtggtcggcatcatgtgtgtggtggcctactgcaaaaccaagaagcagcggcagaagcttcatgatcggcttcggcagagtcttcggtcagaacggagcaacctggtgaacatagcgaatgggcctcaccacccaaacccaccgccagagaacgtgcagctggtgaatcaatacgtatctaaaaacgtcatctccagtgagcatattgttgagagagaagtggagacttccttttccaccagtcattacacttccacagcccatcactccacgactgtcacccagactcctagtcacagctggagtaatgggcacacggagagcgtcatttcagaaagcaactccgtaatcatgatgtcttcggtagagaacagcaggcacagcagtcccgccgggggcccacgaggacgtcttcatggcctgggaggccctcgtgataacagcttcctcaggcatgccagagaaacccctgactcctacagagactctcctcatagcgaaaggtatgtatcagccatgaccaccccggctcgtatgtcacctgtagatttccacacgccaagctcccctaaatcgcccccttcggaaatgtctccacccgtgtccagcatgacggtgtccatgccctctgtggcagtcagcccctttgtggaagaagagaggcctctgctgcttgtgacgccaccaaggctacgggagaagaaatatgatcatcacccccagcaactcaactcctttcatcacaaccctgcacatcagagtaccagcctcccccctagcccactgaggatagtggaggatgaggagtacgagacgacccaggagtatgagtcagttcaagagcccgttaagaaagtcaccaatagccggcgggccaaaagaaccaagcccaatggccacattgccaataggttggaaatggacagcaacacaagttctgtgagcagtaactcagaaagtgagacagaagacgaaagagtaggtgaagacacaccattcctgggcatacagaaccccctggcagccagccttgaggtggcccctgccttccgtctggctgagagcaggactaacccagcaggccgcttctccacacaggaggaattacaggccaggctgtctagtgtaatcgctaaccaagaccctattgctgtataaaacctaaataaacacatagattcacctgtaaaactttattttatataataaagtatttcaccttaaattaaacaatttattttattttagcagttctgcaaatagaaaacaggaagaaaaaaaaacttttataaattaaatatatgtatgtaaaaatgtgttatgtgccatatgtagcaatttttttacagtatttcaaaaacgagaaagatatcaatggtgcctttatgttctgttatgtcgagagcaagttttataaagttatggtgatttctttttcacagtatttcagcaaaacctcccatatattcagtttctgctggctttttgtgcattgcattatgatgttgactggatgtatggtttgcaaggctagcagctcgctcgtgttctctctctctctctctctctctctctctctgtctctctctctgtctctctctctctctctctctctctctctctctctctctctctctctctctctctgtctctctctctgcttcccgtagctcccaaccagtactgtcttggactggcacatccatccaaatacctttctactttgtatgaagttttctttgctttcccaatatgaaatgagttctctctactctgtcagccaaaggtttgcttcactggactctgagataatagtagacccagcagcatgctactattacgtatagcaggaaactgcaccaagtaatgtccaataataggaagaaagtaatactgtgatttaaaaaaaaaaacaaactatattattaatcagaagacagcttgctcttggtaaaaggagctaccattgactctaattttgactttttagttattgttcttgacaaagagtaacagcttcaagtacagcctagaaaaaaaaatgggttctggcctgctatcaggataaatctatcgacgtagatagattcaactcagtttcactttctgtcttgggggaaatgatccagccactcatatgacgaccaaccaaccacaggtgcctctgctccctgt

SEQ ID NO: 2 - DNA, 2103 bp, Mus musculus
atggagatttatcccccagacatgtctgagggagctggcgggaggtcctccagcccctccactcagctgagtgcagacccatctctcgatgggcttccggcagcggaacatatgccagacacccacacagaagatgggagaagccctggactcctgggcctggccgtgccctgctgtgtctgcctggaagcggagcgtctcagagggtgcctcaactccgagaagatctgcattgttcccattctggcttgtctagtaagcctctgcctctgcattgctggcctaaagtgggtatttgtggacaagatattcgaatacgactctcctacccaccttgaccctggggggttaggccaggaccctgttatttctctggatccaacggctgcctccgctgttctggtctcatccgaggcatacacttcacctgtctctaaggctcagtctgaagccgaggctcatgttacagggcaaggtgaccatgtcgctgtggcctctgaaccttccgcagtacccacccggaagaaccggctgtctgcttttcctcccttacactccactccaccgcccttcccttctccagctcggacccctgaggtgagaacacccaagtcaggaactcagccacaaacaacagaaactaatctgcaaactgctcctaaactttccacatctacatccacgactgggaccagccatctcataaagtgtgcggagaaggagaaaactttctgtgtgaatggaggcgagtgcttcatggtgaaggacctgtcaaacccctcaagatacttgtgcaagtgcccaaatgagtttactggtgatcgttgccaaaactacgtaatggccagcttctacaagcatcttgggattgaatttatggaagcggaggagctctaccagaagagggtactgacaattactggcatctgtatcgccctgttggtggtcggcatcatgtgtgtggtggcctactgcaaaaccaagaaacagcggcagaagcttcatgatcggctccggcagagccttcggtcagaacgaaacaacatggtgaacatagcgaatggccctcaccatccaaacccaccaccagagaatgtgcaactggtgaatcaatatgtatctaaaaacgtcatctccagtgagcatattgtggagagagaagtggagacctccttttccaccagtcactacacttccacagctcatcactccacgactgtcacccagactcctagtcacagctggagtaatgggcacacagaaagcatcatttcagaaagccactctgtaatcatgatgtcatcggtagagaacagcaggcacagcagcccagctgggggcccacgaggacgtcttcatggcctgggaggccctcgcgaatgtaacagcttcctcaggcatgccagagaaacccctgactcctacagagactctcctcatagtgaaaggtatgtatcagccatgaccaccccggctcgtatgtcacctgtagatttccacacgccaagctcccctaaatcgcccccttcggaaatgtctccacccgtgtccagcatgacggtgtccatgccctctgtggcagtcagcccctttgtggaagaagagaggcctctgcttcttgtgacgccaccgaggctacgggagaagaagtatgatcatcacccccagcaactcaactcctttcatcacaaccctgcacatcagagtaccagcctcccccctagcccattgaggatagtggaggatgaggaatacgaaacgacccaggagtatgagccaattcaagagcctattaagaaagtcaccaatagccggcgggccaaaagaaccaagcccaatggccacattgccaataggttggaaatggacagcaacccaagttctgtgagcagtaactcagaaagtgagacagaagatgaaagagtaggtgaagatacaccattcctgggcatacagaaccccctggcagccagccttgaggtggcccctgccttccgtctggctgagagcaggactaacccagcaggccgcttctccacacaggaagaattacaggccaggctgtctagtgtaatcgctaaccaagaccctattgctgtataa

SEQ ID NO: 3 - DNA, 3270 bp, Rattus norvegicus
cagctgcgctagcaccaggcacagcactggtgccacgcgcccgccgagcccaccgcggtcacttcagccaccagattgcaactttgcggagatgatggactagcatggcctgaagcatggctcagttctattacaaacgaaatgtcaacgccccctaccgagaccgcatcccactgaggatcgtcagggcagaatctgaactctcaccatcagagaaagcctacttgaatgccgtggaaaagggggactatgcaagcgtcaagaaatctctggaggaagccgagatttattttaaaatcaacattaactgcattgacccccttgggaggactgctcttctcattgccattgaaaatgagaacctggagctgattgaactgttgttgagtttcaatgtctatgttggcgatgcgctacttcacgccatcaggaaagaggtggttggagccgtggagctactgctgaaccacaaaaaagcccagcggagagaagcaggtgcctcccatcctccttgacaaacagttctctgaattcaccccagacatcacgcctatcatcttggctgcacatacaaataattatgagataatcaaactcttggtccagaagggtgtctcggtgcccagaccccacgaggtccgctgtaactgtgttgagtgtgtctccagctcagacgtggacagcctcaggcactcacggtccaggctcaacatctacaaggctttggccagcccctcgctcattgcgctgtcaagtgaagaccctttcctcaccgcctttcagttaagctgggagctgcaagaactgagtaaggtggagaatgaattcaagtcggagtatgaggagctgtctagacagtgcaaacagtttgctaaggacctcctagatcagacacggagttccagagagctggaaatcattcttaattaccgtgatgacaatagcctgatcgaagaacagagtggaaatgatcttgcgaggctaaaattagccattaagtaccgtcaaaaagagtttgttgctcagcccaactgccagcagctgcttgcttcccgctggtacgatgagttcccaggctggaggagaagacactgggcggtgaagatggtgacatgtttcataataggactactcttccccgtcttctccgtgtgctacctgatagctcccaaaagcccacttggactgttcatcagaaagccatttatcaagtttatctgccacacagcctcctatctgacctttttgtttctgctgctgctagcctctcagcacatcgacaggttttatatggggagagattaaacagatgtgggatggcggacttcaggattacatccacgactggtggaatctaatggactttgtgatgaactccttgtatctggcgacaatctccttgaagattgtcgcatttgtaaagtacagtgctctgaacccacgggaatcatgggacatgtggcaccccaccctggtggcagaggctttattcgcaattgcaaacatcttcagttccctccgcctgatctctctgttcactgccaattctcacctggggcctctgcagatatctctgggaagaatgctcctggacatcctaaagttcttattcatatactgcctcgtgctgctagcttttgcaaatggcctaaatcaactgtacttctactatgaagaaacgaaggggttaagctgcaaaggcatacggtgcgaaaaacagaacaacgcgttctccacgttatttgagactctacagtccctgttttggtcaatatttggactcatcaatctctatgttaccaatgtcaaagcccagcatgagttcactgagtttgttggggccaccatgtttggcacatataacgtcatctctctggttgtcctcctgaacatgctgatcgctatgatgaataattcttaccaactaattgccgaccacgcagatatagagtggaaatttgctcgaacaaagctttggatgagctactttgaagaagggggtaccctgcctacacctttcaatgtcatcccaagccccaagtccctgtggtacctggtcaagtggatatggacgcacttatgtaagaaaaagatgagaagaaagccagaaagctttgggacaattgggcggcgtgctgctgataacttgagaaggcatcaccaataccaagaggtgatgaggaatctggtgaagcggtacgtggcagccatgatcagagaggcaaaaactgaagaaggcttgacagaggagaatgttaaggaactaaagcaagacatttctagcttccgcttcgaagttctgggattgctccggggaagcaagctctcaacaatacagtcagccaacgcagcgagttcagccagctccgcggactccgatgagaagagccacagcgaaggtaatggcaaggacaagagaaagaatctcagcctctttgatttaaccactctgatccacccgcggtcggcagtcattgcctccgagagacataacctaagcaatggttctgccctggtggtgcaggagccgcccagggagaagcagaggaaagtgaattttgtggctgatatcaaaaacttcgggttatttcatagacggtcaaagcaaaatgctgctgagcaaaacgcaaaccaaatcttctctgtttcagaagaaattactcgtcaacaggcggcaggagcacttgagagaaatatccaactggaatcccaaggattagcttcacggggtgaccgcagcattcctggtctcaatgaacagtgtgtgctagtagaccatagagaaaggaatacggacactttgggtttacaggtaggcaagagagtgtgctcctccttcaagtcggagaaggtggtggtggaagacaccgtccctattataccaaaggagaaacacgcccaggaggaggactcaagcatagattatgatttaagccccacggacacagttgcccatgaagattatgtgaccacgagattgtgacaacttggagaaggagtgtttaccatacctatacatattttccatagtgctctgggcaggcaaaatgtatgaaattacattatcaaatgctaatttacactttctaacgtttatctgtcgtggcgtattagcctgtatttatgtttgaacaaagcagaggcaacgtgaaccctcctcttttgtagcctgcttttgctatcatggtttattttacaagtgtttctgttgaataaacgcaccttctacccttgtactgttacaataacccacagaaaatttttagctat

SEQ ID NO: 4 - DNA, 3422 bp, Mus musculus
gtttttttcccccttggaatgctccaaaaaactcggtagcgactacggaaaccccatcggaactgaccagctgcgctagcaccaggcacagcactggtgctgcgcgctcgccgagcccacctcggtcacttcaaccaccagattgcaactttgcggagatgatgatggactagcatggcctgaagcatggctcagttctattacaaaagaaatgtcaacgccccctacagagaccgcatcccactgaggattgtcagagcagaatctgagctctcaccatcagagaaagcctacttgaatgctgtggagaagggggactatgcaagcgtcaagaagtctctggaggaagctgagatttattttaaaatcaacattaactgcatcgaccccctgggaaggaccgccctcctcattgccattgaaaatgagaatctggagcttattgaactattgttgagtttcaatgtctatgtaggcgatgcgctgcttcacgccatcagaaaagaggtggttggagccgtggagctactgctgaaccacaaaaagccaagtggagagaagcaggtgcctcccattctccttgataaacagttctctgaattcactccggacatcacacccatcatcttggctgcacatacaaataattacgagataatcaaacttttggttcagaaaggtgtctcagtgcccagaccccacgaggtccgctgtaactgtgttgagtgtgtctccagctcggatgtggacagcctcaggcattcacggtccaggctcaacatctacaaggccttggccagcccctcgctcattgccctgtcaagcgaagaccctttccttactgcctttcagttaagttgggagctgcaagaactcagcaaggtggagaacgaattcaagtcggagtatgaggagctgtctagacagtgcaaacaatttgccaaggacctcctagatcagacacggagttccagagagctggaaatcattcttaattaccgtgatgacaatagtctgatcgaagaacagagtggaaatgatcttgcaaggctaaaattagccattaagtaccgtcaaaaagagtttgttgctcagcccaactgccagcagctgctcgcttcccgctggtacgatgagttcccaggctggaggagaagacactgggcggtgaagatggtgacgtgtttcataataggactactcttccccgtcttctccgtgtgctacctgatagctcccaaaagcccacttggactgttcatcagaaagccatttatcaagtttatctgccacacagcctcctatctgacctttttgtttctgctgctgctagcctctcagcacatcgacaggtcagacttgaacaggcaaggtccaccaccaaccatcgtggagtggatgatattaccgtgggtcctgggttttatatggggagagattaaacagatgtgggatggcggactccaggattacatccatgactggtggaatctaatggactttgtgatgaactccttgtatctggcaacaatctccttgaagattgtcgcgtttgtaaagtacagtgctctgaacccacgggaatcatgggacatgtggcaccccaccctggtggcagaggcattatttgctattgcaaacatcttcagttccctccgcctgatctctctgttcactgccaattctcacctggggcctctgcagatatctctgggaaggatgcttctggacatcctgaagttcttgttcatctactgcctcgtgctgctagcttttgcaaatggcctaaatcagctgtacttttactatgaagaaacaaaggggctaagctgcaaaggcatccggtgcgagaaacagaacaacgcgttttccacgttattcgagacactacagtccctgttttggtcaatatttggactcatcaatctctatgttaccaatgtcaaagcccagcacgagttcactgagtttgttggggccaccatgtttggcacatataatgtcatctctctggttgtcctgctgaacatgttaattgctatgatgaataattcttaccaactaattgccgaccatgcagatatagaatggaaatttgctcgaacaaagctttggatgagctactttgaagaaggaggtaccctgcctacacctttcaatgtcatcccaagccccaagtccctgtggtacctggtcaagtggatatggacacacttatgtaagaaaaaaatgagaaggaagccagaaagcttcgggacaattgggcggcgtgctgctgataacttgagaagacatcaccaataccaagaggtgatgaggaacctggtgaagcggtacgtggcagccatgatcagagaggcaaaaaccgaagaaggcttgacggaggagaatgttaaggaactaaagcaagacatttctagcttccgcttcgaagttctgggattgctcagaggaagcaagctctctacaatacagtcagccaacgcggcgagttcagcggactccgacgagaagagccagagcgaaggtaatggcaaggacaagagaaagaatctcagcctctttgatttaaccactctgatccacccgcggtcggcagccattgcctccgagagacataacctaagcaatggttccgccctggtggtgcaggagccgcccagggagaagcagaggaaagtgaattttgtggctgatatcaaaaacttcgggttatttcatagacggtcaaaacaaaatgctgctgagcaaaacgcaaaccaaatcttctctgtttcagaagaaattactcgtcaacaggcggcaggagcacttgagcgaaatatcgaactggaatccaaaggattagcttcacggggtgaccgcagcattcctggtctcaatgaacagtgtgtgctagtagaccatagagaaaggaatacggacactttgggtttacaggtaggcaagagagtgtgctccaccttcaagtcggagaaggtggtggtggaagacaccgtccctattataccaaaggagaaacacgcccacgaggaggactcgagcatagactatgacttaagccccacggacacagctgcccacgaagattatgtgaccacaagattgtgacccttggaggagtgtttaccatacctatacatattttccatagtgctctgggcaggcaaaatgtttgaaatcccattatcaaatgctaatttccactttctaatgtttatctgttgtggcatattaacctgtaatatgtttgaacaaagcagaagtaatatgaaccctcctcttttgtagcctgcttttgctttcaccgttgattttacaagtgtttctgttaaataaacgcaccttttatccttgtactgttacaataacccacagaaaatttttagctatctttttcaattaaaaccaatgcaattgtt

SEQ ID NO: 5 - DNA, 4060 bp, Rattus norvegicus
aattgtcagcacgaattctgagacttgccaaaaatgaagctggcgacgggactgtgggtctgggggagccttctggtggcagccaggaccgtccagcccagcgcttctcagtcagtgtgtgccggaacagagaacaaactgagctctctctctgatctggagcagcagtaccgagccttgcgcaaatactatgaaaactgcgaggtagtcatgggcaacctggagatcaccagcatagagcacaaccgggacctctccttcctgcggtctatccgagaagtcacaggctatgtacttgtggccctcaaccagtttcgttacctgcctctggagaatttacgcattattcgtgggacaaaactgtatgaagatcgctatgccttagcaatattcttaaactacaggaaagatggcaactttggacttcaagaactgggattaaagaacctgaccgaaatactaaatggtggagtctatgtagaccagaacaaattcctatgttatgctgatactatacactggcaagatattgttcggaatccatggccttccaacatgactctggtgtcaacaattggaagttctggatgcggaagatgccataagtcttgcactggtcgatgctggggacccacagaaaatcactgccagaccttgacaaggactgtgtgtgcagaacaatgtgatggcaggtgctatggaccctacgtcagtgactgctgccatcgagaatgtgccggaggctgctcaggaccaaaagacactgactgctttgcctgcatgaacttcaatgacagtggagcatgtgttactcagtgtccccaaacgttcgtctacaatccaaccacctttcaactggaacacaacttcaatgcaaagtacacatatggagcattctgtgttaagaaatgtccacataacttcgtggtagattccagttcttgtgtacgagcctgccctagttccaagatggaagtcgaagaaaatggaattaaaatgtgtaagccttgcactgatatttgccccaaagcatgtgatggaatcggcaccggatccttgatgtctgctcagactgtggattccagtaacattgacaaattcataaactgcaccaagatcaacgggaatctcatctttcttgtcactggcattcatggggacccttacaatgctattgacgccatagacccagagaaactgaatgtctttcggacagtcagagaaataacaggtttcctgaacatacagacttggcccccaaatatgacagatttcagtgttttctccaaccttgtaaccattggaggaagagtcctctacagtggtctgtcattgcttatcctcaaacaacaaggtatcacttcactacagttccagtctctgaaggaaatcagtgcgggcaatatctacatcacggacaacagcaacctgtgttattaccacaccatcaactggacaacactgttcagcaccgttaaccagaggatagtgatccgagacaacaggagggctgagaactgtactgctgaagggatggtgtgtaaccacctgtgttcaaatgatggttgttggggacctgggccagaccagtgtctgtcatgtcggcgcttcagcaggggaaagatctgtatagagtcctgcaacctttatgatggggagtttcgagagtttgaaaatggctccatctgtgttgagtgtgactcccagtgtgagaaaatggaagacggactcctcacatgccatggaccgggacctgacaactgtacaaagtgttctcattttaaagatggtccaaactgtgtagagaaatgtccagatgtcctacagggagcaaacagtttcatatttaagtacgcagatcaggatcgggagtgccacccttgccatccaaactgcacccaggggtgtaacggtcccactagtcatgactgcatttactacccatggacgggccattccactttaccacaacatgctagaactccactgattgcagccggagtcattggtgggctcttcatcctggtcatcatggctctgacatttgccgtttatgtcagaaggaagagcatcaaaaagaaacgcgctttgagaagattcctggagaccgagttagtcgagcccttaacccctagtggcacagcacccaatcaagctcaacttcgaattttgaaggaaacagagctaaagagggtaaaagtccttggctcgggagcatttggaaccgtttataaaggaatctgggtacctgaaggagaaactgtgaaaatccctgtggctattaagatcctcaatgagacaactggccccaaagccaatgtggagttcatggatgaggcactgattatggcaagtgtggatcacccacacctagtgcgtttactgggtgtgtgtttgagccccactatccagttggttactcagttgatgccacatggctgcctactggaatatgtccacgaacacaaggataacatcggatcacaactgctgttgaactggtgtgtccagattgctaagggaatgatgtatctggaggaaaggcggcttgttcatcgggatctggcagcccgcaatgtgttggtgaaatctccaaatcatgttaaaatcacagactttggactggcccggctcttggaaggagatgaaaaagaatacaatgctgacggtggcaagatgccaattaaatggatggctctggaatgtatacattataggaaattcacacatcaaagcgatgtttggagctacggtgtcactatatgggaactgatgacctttggaggaaagccctatgatggaattccaacgcgagaaatccctgatttattagagaagggagagcgtttgcctcaacctcccatctgcactattgacgtttacatcgtcatggtcaaatgttggatgatcgatgctgacagcagacctaaattcaaagaactggctgctgagttttcaaggatggctagagaccctcaaagatacctagtaattcagggggatgatcgcatgaagcttcccagtccaaacgacagcaaattcttccagaatctcttggatgaagaggatttggaagatatgatggacgctgaggaatatttggtcccccaggctttcaatatcccacctcctatctacacatccagaacaagaattgactccaataggagtgaaattggacacagccctcctcctgcctacacccccatgtcgggaagtcagtttgtgtaccaggatgggggtttcgctacacaacaaggaatgcccatgccctacacagccacaaccagcaccataccagaggctccagtcgcccagggtgcaacggctgagatgtttgatgactcctgctgtaatggtaccctgcgaaagccagtggtaccccacgtccaagaggacagtagcactcagaggtatagtgccgaccccacagtgttcgccccagaacggaacccacgagcagaactggatgaagaaggctacatgactcccatgcatgacaagccaaaacaagaatatctgaatcctgtggaagagaacccttttgtgtcccggaggaagaatggagaccttcaagctttagataatccagagtatcacagcgcttccagcggtccccccaaggcagaggatgagtacgtgaatgagcccctttatctcaacaccttcaccaacgccttgggaaatgcagagtacatgaaaaacagcttactgtctgtgccagagaaagccaagaaagcatttgacaaccccgactactggaaccacagcctgccaccccggagcactcttcagcacccagactacctgcaggaatacagcacaaaatatttttataaacagaatggacggatccgccctattgtggcagagaatcctgagtacctctcagagttctcgctgaagccaggcactatgctgccccctccgccctacagacaccggaatactgtggtgtgagctcagctagagtgttttaggagcagaaacacacccgctccatttccccttctccctcctctttctctggcagtcttccttctaccccaaggccagtagt

SEQ ID NO: 6 - DNA, 4257 bp, Mus musculus
actccgggaactagctgtacgttgtgctcggagcaccagccgcacagtcgcgctcactcccacccgcgcgccctcctccgcggccccttgccgggtccgcgggtccacgggtcctggaagccgccgccgtcgccgactggctctccggccccgggaagcccgtgcaccaagcgcgccgcgcccgccccccttgcgccccccacgcgctcccggctgagggggggagatctcctccgcgtgctcgcaagtggctatggtatttggacatgtaattgtcagcgcgggatctgagacttgccaaaaatgaagctggcgacgggactctgggtctgggggagccttctgatggcagcggggaccgtccagcccagcgcttctcagtcagtgtgcgcaggaacagagaacaaactgagctctctctctgacctggaacagcagtaccgagccttgcgcaaatactatgaaaactgcgaggtagtcatgggcaacctggagatcaccagcatcgagcacaaccgggacctctccttcctgcggtctatccgagaagtcacaggctacgtcctggtggccctcaaccagtttcgttacttgcctctggagaatttacgcattattcgtgggacaaaactatatgaagatcgctatgccttagcgatattcttaaactacaggaaagatggcaactttggactccaagaacttggattaaagaacctgaccgaaatactaaatggtggagtctatgtagaccagaacaaattcctatgttatgctgacactatacactggcaagatattgttcggaatccatggccttccaacatgactctggtgtcaacaaatggaagttctggatgtggaagatgccataagtcttgcactggccgatgctggggacccacagaaaatcactgccagaccttgaccagaactgtgtgtgctgaacaatgtgatggcaggtgctatggaccctacgttagtgactgctgccatcgagaatgtgctggaggctgctcaggaccaaaggacactgactgctttgcctgcatgaacttcaatgacagtggagcctgcgttactcaatgtccccaaacatttgtctacaatccaaccacctttcaactggaacacaacttcaatgcaaagtacacgtatggagcattctgtgttaagaaatgtccacataacttcgtggtagattccagttcttgtgtacgagcctgccctagttctaagatggaagtagaagaaaatgggattaaaatgtgtaagccttgcaccgatatttgccccaaagcatgtgatggaatcggcacgggatcactgatgtctgctcagactgtggattcaagtaacattgacaaattcataaactgcacaaagatcaatggcaatctcatctttcttgtcactggcattcatggagacccttacaatgctattgacgccatagatccagagaaactgaatgtctttcggactgtcagagaaataacaggtttcctgaacatacagtcttggcccccaaatatgacagatttcagtgttttctccaacctcgtcacaattggaggaagagtcctctacagtggtctctcattgctgatcctcaaacaacaaggtatcacttccctacagttccagtctctgaaggaaatcagtgcgggcaatatctacatcactgacaacagcaacctgtgttattaccataccattaactggacaacactcttcagcaccattaaccagagaatagtgatccgagataacagaagagctgagaattgtactgctgaaggcatggtatgcaaccacctgtgttcaaatgatggttgttggggacctgggccggaccagtgcctgtcatgtcggcgcttcagcaggggaaagatctgcatagagtcttgcaacctttatgatggggaatttcgagagtttgaaaacggctccatctgtgttgagtgtgactcccagtgtgagaaaatggaagatggactcctcacatgccatggaccgggacctgacaactgcacaaagtgctctcattttaaggatggtccaaactgtgtggagaaatgtccagatggcctacagggagcaaacagtttcatttttaagtatgcagatcaggatcgggagtgccacccttgccatccaaactgcacccaggggtgtaacggtcccactagtcatgactgcatttactacccatggacgggccattccactttaccacaacacgctagaactccactgattgcagccggagtcattggaggcctcttcatcctggtgatcatggctttgacatttgctgtctatgtcagaagaaagagcatcaaaaagaaacgtgctttgaggagattcctggagacagagctggtagagcccttaactcccagtggcacggcacccaatcaagctcaacttcgcattttgaaggaaaccgaactaaagagggtaaaggtccttggctcgggagcttttggaaccgtttataaaggtatttgggtgcctgaaggtgaaacagtgaaaatccctgtggctataaagatcctcaatgaaacaactggccccaaagccaacgtggagttcatggatgaggctctgatcatggcaagtatggatcacccacacctagttcgcctattgggagtgtgtctgagtcccactatccagttggttacgcagctgatgccgcatggctgcctactggactatgttcatgaacacaaggataacattggatcacagctgctgttgaactggtgtgtccagattgctaagggaatgatgtacctagaagaaagacggcttgttcatcgggatctggcagcccgcaatgtcttagtgaaatctccaaatcatgttaaaatcacagattttggactggcccggctcttggaaggagatgaaaaagaatacaatgctgatggtggcaagatgccaattaaatggatggctctggaatgtatacattataggaaattcacacatcaaagtgatgtttggagctatggcgtcactatatgggaactgatgacctttggaggaaagccctatgatggaattccaacccgagaaatccccgatttactggagaaaggagagcgtctgcctcagcctcccatctgcactattgatgtttacatggtcatggtcaaatgttggatgatcgatgctgacagcagacctaaattcaaagaactggctgctgagttttcaagaatggctagagaccctcaaagatacctagttattcagggtgatgatcgtatgaagcttcccagtccaaatgacagcaaattcttccagaatctcttggatgaagaggatttggaagacatgatggatgctgaggaatatttggtcccccaggctttcaacatacctcctcccatctacacatccagaacaagaattgactccaataggaatcagtttgtgtaccaagatgggggctttgctacacaacaaggaatgcccatgccctacagagccacaaccagcaccataccagaggctccagtagctcagggtgcaacggctgagatgtttgatgactcctgctgtaatggtaccctacgaaagccagtggcaccccatgtccaagaggacagtagcactcagaggtatagtgctgatcccacagtgttcgccccagaacggaatcctcgaggagaactggatgaagaaggctacatgactccaatgcatgacaagcccaaacaagaatatctgaatcctgtggaagagaacccttttgtgtcccgaaggaagaatggagatcttcaagctttagataatccggagtatcacagtgcttccagcggtccacccaaggcggaggatgaatacgtgaatgagcctctatacctcaacaccttcgccaatgccttggggagtgcagagtacatgaaaaacagtgtactgtctgtgccagagaaagccaagaaagcatttgacaaccccgactactggaaccacagcctgccaccccggagcacccttcagcacccagactacctgcaggaatacagcacaaaatatttttataaacagaatggacggatccgccccattgtggcagagaatcctgagtacctctcggagttctcgctgaagcctggcactatgctgccccctccgccctacagacaccggaatactgtggtgtgagcttggctagagtgttaggtggagaaacacacacccactccatttcccttccccctcctctttctctggtggtct

SEQ ID NO: 7 - DNA, 312 bp, Artificial Sequence
  Sequence Tag
tacatatacatatacatatacatatacatatacatatacatatacatatacatatacatatacatatacatatacatcatatacatatacccagagagagggagatagtgcatatacatatagtgtttttatcaattgattacaatttcataattatccttattcacaaagtcatgcattatgactatattcactttccattcctcctccaaaacctcccagctccagtcctaccccctaacttgctctcaatttcatgtctgcttttgtttccttatcactataaaccaccaagtcagcttctactcctag

SEQ ID NO: 8 - DNA, 168 bp, Artificial Sequence
  Sequence Tag
tatgtttaggccatggagataagaggcatcttccagagttaggaattacatacatctgcacttatgtatcacgattatgcttctgaatgcacctaacaagagctcgaggagaaaccatgcagagaggaacaattgaaaaggaagtacattgtgcagactgcttcctag

SEQ ID NO: 9 - DNA, 37 bp, Artificial Sequence
  Sequence Tag
tacatccatgtttttctactgatgtccttgtctctag

SEQ ID NO: 10 - DNA, 312 bp, Artificial Sequence; identical to SEQ ID NO: 7
  Sequence Tag

SEQ ID NO: 11 - DNA, 168 bp, Artificial Sequence; identical to SEQ ID NO: 8
  Sequence Tag

SEQ ID NO: 12 - DNA, 1023 bp, Artificial Sequence
  SB Transposase
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 13 - DNA, 229 bp, Artificial Sequence
  SB 5′ ITR
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 14 - DNA, 229 bp, Artificial Sequence
  SB 3′ ITR
attgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 15 - DNA, 1785 bp, Artificial Sequence
  PB Transposase
atgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctga

SEQ ID NO: 16 - DNA, 309 bp, Artificial Sequence
  PB 5′ ITR
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 17 - DNA, 238 bp, Artificial Sequence
  PB 3′ ITR
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg